(19) United States
(12) Patent Application Publication
(10) Pub. No.: US 2010/0331390 A1
(43) Pub. Date: Dec. 30, 2010

(54) EFFECTS OF APOLIPOPROTEIN B INHIBITION ON GENE EXPRESSION PROFILES IN ANIMALS

(75) Inventors: Rosanne M. Crooke, Carlsbad, CA (US); Mark J. Graham, San Clemente, CA (US)

Correspondence Address: JONES DAY, 222 EAST 41ST ST, NEW YORK, NY, 10017, US

(73) Assignee: Genzyme Corporation, Cambridge, MA (US)

(21) Appl. No.: 12/720,581

(22) Filed: Mar. 9, 2010

Related U.S. Application Data
(63) Continuation of application No. 11/123,656, filed on May 5, 2005, now abandoned.
Application No. 11/123,656 is a continuation-in-part of application No. 10/712,795, filed on Nov. 13, 2003, now Pat. No. 7,511,131.
(60) Provisional application No. 60/568,825, filed on May 5, 2004.

(51) Int. Cl. A61K 48/00; C07H 21/04; C12N 5/02; A61P 3/04; A61P 9/00

(52) U.S. Cl. 514/44 A; 536/24.5; 435/375

(57) ABSTRACT

Methods are provided for modulating the expression of genes involved in lipid metabolism, useful in the treatment of conditions associated with cardiovascular risk. Antisense oligonucleotides targeted to apolipoprotein B reduce the level of apolipoprotein B mRNA, lower serum cholesterol and shift liver gene expression profiles from those of an obese animal towards those of a lean animal. Further provided are methods for improving the cardiovascular risk of a subject through antisense inhibition of apolipoprotein B. Also provided are methods for employing antisense oligonucleotides targeted to apolipoprotein B to modulate a cellular pathway or metabolic process.

### CROSS REFERENCE TO RELATED APPLICATIONS

This application is a continuation of U.S. application Ser. No. 11/123,656, filed May 5, 2005, which is a continuation-in part of U.S. application Ser. No. 10/712,795, filed Nov. 13, 2003, and claims the benefit of priority of U.S. provisional application No. 60/568,825, filed May 5, 2004, each of which is incorporated by reference herein in its entirety.

### SEQUENCE LISTING

A paper copy of the sequence listing and a computer-readable form of the sequence listing, on diskette, containing the file named BIOL0039USSEQ.txt, which is 26,112 bytes and was created on May 5, 2005, are herein incorporated by reference.

### FIELD OF THE INVENTION

The present invention provides methods for modulating the expression of genes involved in lipid metabolism. In particular, this invention relates to the modulation of such genes following the antisense inhibition of apolipoprotein B, which has been shown to improve lipid profiles in animals. The invention also provides methods lowering the cardiovascular risk profile of an animal.

### BACKGROUND OF THE INVENTION

Lipoproteins are globular, micelle-like particles that consist of a non-polar core of acylglycerols and cholesteryl esters surrounded by an amphiphilic coating of protein, phospholipid and cholesterol. Lipoproteins have been classified into five broad categories on the basis of their functional and physical properties: chylomicrons, which transport dietary lipids from intestine to tissues; very low density lipoproteins (VLDL); intermediate density lipoproteins (IDL); low density lipoproteins (LDL); all of which transport triacylglycerols and cholesterol from the liver to tissues; and high density lipoproteins (HDL), which transport endogenous cholesterol from tissues to the liver.

Lipoprotein particles undergo continuous metabolic processing and have variable properties and compositions. Lipoprotein densities increase without decreasing particle diameter because the density of their outer coatings is less than that of the inner core. The protein components of lipoproteins are known as apoliproteins. At least nine apolipoproteins are distributed in significant amounts among the various human lipoproteins.

Apolipoprotein B (also known as ApoB, apolipoprotein B-100; ApoB-100, apolipoprotein B-48; ApoB-48 and Ag(x) antigen), is a large glycoprotein that serves an indispensable role in the assembly and secretion of lipids and in the transport and receptor-mediated uptake and delivery of distinct classes of lipoproteins. The importance of apolipoprotein B spans a variety of functions, from the absorption and processing of dietary lipids to the regulation of circulating lipoprotein levels (Davidson and Shelness, Annu. Rev. Nutr., 2000, 20, 169-193). This latter property underlies its relevance in terms of atherosclerosis susceptibility, which is highly correlated with the ambient concentration of apolipoprotein B-containing lipoproteins (Davidson and Shelness, Annu. Rev. Nutr., 2000, 20, 169-193).

Two forms of apolipoprotein B exist in mammals. ApoB-100 represents the full-length protein containing 4536 amino acid residues synthesized exclusively in the human liver (Davidson and Shelness, Annu. Rev. Nutr., 2000, 20, 169-193). A truncated form known as ApoB-48 is colinear with the amino terminal 2152 residues and is synthesized in the small intestine of all mammals (Davidson and Shelness, Annu. Rev. Nutr., 2000, 20, 169-193).

ApoB-100 is the major protein component of LDL and contains the domain required for interaction of this lipoprotein species with the LDL receptor. In addition, ApoB-100 contains an unpaired cysteine residue which mediates an interaction with apolipoprotein(a) and generates another distinct atherogenic lipoprotein called Lp(a) (Davidson and Shelness, Annu. Rev. Nutr., 2000, 20, 169-193).

In humans, ApoB-48 circulates in association with chylomicrons and chylomicron remnants and these particles are cleared by a distinct receptor known as the LDL-receptor-related protein (Davidson and Shelness, Annu. Rev. Nutr., 2000, 20, 169-193). ApoB-48 can be viewed as a crucial adaptation by which dietary lipid is delivered from the small intestine to the liver, while ApoB-100 participates in the transport and delivery of endogenous plasma cholesterol (Davidson and Shelness, Annu. Rev. Nutr., 2000, 20, 169-193).

The basis by which the common structural gene for apolipoprotein B produces two distinct protein isoforms is a process known as RNA editing. A site specific cytosine-to-uracil editing reaction produces a UAA stop codon and translational termination of apolipoprotein B to produce ApoB-48 (Davidson and Shelness, Annu. Rev. Nutr., 2000, 20, 169-193).

Apolipoprotein B was cloned in 1985 (Law et al., Proc. Natl. Acad. Sci. U.S.A., 1985, 82, 8340-8344) and mapped to chromosome 2p23-2p24 in 1986 (Deeb et al., Proc. Natl. Acad. Sci. U.S.A., 1986, 83, 419-422).

Disclosed and claimed in U.S. Pat. No. 5,786,206 are methods and compositions for determining the level of low density lipoproteins (LDL) in plasma which include isolated DNA sequences encoding epitope regions of apolipoprotein B-100 (Smith et al., 1998).

Transgenic mice expressing human apolipoprotein B and fed a high-fat diet were found to develop high plasma cholesterol levels and displayed an 11-fold increase in atherosclerotic lesions over non-transgenic littermates (Kim and Young, J. Lipid Res., 1998, 39, 703-723; Nishina et al., J. Lipid Res., 1990, 31, 859-869).

In addition, transgenic mice expressing truncated forms of human apolipoprotein B have been employed to identify the carboxyl-terminal structural features of ApoB-100 that are required for interactions with apolipoprotein(a) to generate the Lp(a) lipoprotein particle and to investigate structural features of the LDL receptor-binding region of ApoB-100 (Kim and Young, J. Lipid Res., 1998, 39, 703-723; McCormick et al., J. Biol. Chem., 1997, 272, 23616-23622).

Apolipoprotein B knockout mice (bearing disruptions of both ApoB-100 and ApoB-48) have been generated which are protected from developing hypercholesterolemia when fed a high-fat diet (Farese et al., Proc. Natl. Acad. Sci. U.S.A., 1995, 92, 1774-1778; Kim and Young, J. Lipid Res., 1998, 39, 703-723). The incidence of atherosclerosis has been investigated in mice expressing exclusively ApoB-100 or ApoB-48 and susceptibility to atherosclerosis was found to be dependent on total cholesterol levels. Whether the mice synthesized ApoB-100 or ApoB-48 did not affect the extent of the atherosclerosis, indicating that there is probably no major difference in the intrinsic atherogenicity of ApoB-100 versus ApoB-48 (Kim and Young, J. Lipid Res., 1998, 39, 703-723; Veniant et al., J. Clin. Invest., 1997, 100, 180-188).

Elevated plasma levels of the ApoB-100-containing lipoprotein Lp(a) are associated with increased risk for atherosclerosis and its manifestations, which may include hypercholesterolemia (Seed et al., N. Engl. J. Med., 1990, 322, 1494-1499), myocardial infarction (Sandkamp et al., Clin. Chem., 1990, 36, 20-23), and thrombosis (Nowak-Gottl et al., Pediatrics, 1997, 99, E11).

The plasma concentration of Lp(a) is strongly influenced by heritable factors and is refractory to most drug and dietary manipulation (Katan and Beynen, Am. J. Epidemiol., 1987, 125, 387-399; Vessby et al., Atherosclerosis, 1982, 44, 61-71). Pharmacologic therapy of elevated Lp(a) levels has been only modestly successful and apheresis remains the most effective therapeutic modality (Hajjar and Nachman, Annu. Rev. Med., 1996, 47, 423-442).

Disclosed and claimed in U.S. Pat. No. 6,156,315 and the corresponding PCT publication WO 99/18986 is a method for inhibiting the binding of LDL to blood vessel matrix in a subject, comprising administering to the subject an effective amount of an antibody or a fragment thereof, which is capable of binding to the amino-terminal region of apolipoprotein B, thereby inhibiting the binding of low density lipoprotein to blood vessel matrix (Goldberg and Pillarisetti, 2000; Goldberg and Pillarisetti, 1999).

Disclosed and claimed in U.S. Pat. No. 6,096,516 are vectors containing cDNA encoding murine recombinant antibodies which bind to human ApoB-100 for the purpose of for diagnosis and treatment of cardiovascular diseases (Kwak et al., 2000).

Disclosed and claimed in European patent application EP 911344 published Apr. 28, 1999 (and corresponding to U.S. Pat. No. 6,309,844) is a monoclonal antibody which specifically binds to ApoB-48 and does not specifically bind to ApoB-100, which is useful for diagnosis and therapy of hyperlipidemia and arterial sclerosis (Uchida and Kurano, 1998).

Disclosed and claimed in PCT publication WO 01/30354 are methods of treating a patient with a cardiovascular disorder, comprising administering a therapeutically effective amount of a compound to said patient, wherein said compound acts for a period of time to lower plasma concentrations of apolipoprotein B or apolipoprotein B-containing lipoproteins by stimulating a pathway for apolipoprotein B degradation (Fisher and Williams, 2001).

Disclosed and claimed in U.S. Pat. No. 5,220,006 is a cloned cis-acting DNA sequence that mediates the suppression of atherogenic apolipoprotein B (Ross et al., 1993).

Disclosed and claimed in PCT publication WO 01/12789 is a ribozyme which cleaves ApoB-100 mRNA specifically at position 6679 (Chan et al., 2001).

To date, strategies aimed at inhibiting apolipoprotein B function have been limited to Lp(a) apheresis, antibodies, antibody fragments and ribozymes. However, with the exception of Lp(a) apheresis, these investigative strategies are untested as therapeutic protocols. Consequently, there remains a long felt need for additional agents capable of effectively inhibiting apolipoprotein B function.

Antisense technology is an effective means of reducing the expression of specific gene products and may therefore prove to be uniquely useful in a number of therapeutic, diagnostic and research applications involving modulation of apolipoprotein B expression.

The present invention provides compositions and methods for modulating apolipoprotein B expression, including inhibition of the alternative isoform of apolipoprotein B, ApoB-48.

### SUMMARY OF THE INVENTION

The present invention is directed to compounds, particularly antisense oligonucleotides, which are targeted to a nucleic acid encoding apolipoprotein B. Such compounds modulate the expression of apolipoprotein B and result in a lean animal gene expression profile. Pharmaceutical and other compositions comprising the compounds of the invention are also provided. Further provided are methods of modulating the expression of apolipoprotein B and effecting a lean animal expression profile in cells or tissues comprising contacting said cells or tissues with one or more of the antisense compounds or compositions of the invention. Further provided are methods of treating an animal, particularly a human, suspected of having or being prone to a disease or condition associated with cardiovascular disease by administering a therapeutically or prophylactically effective amount of one or more of the antisense compounds or compositions of the invention.

The present invention provides methods comprising contacting an animal with an antisense oligonucleotide 15-30 nucleobases in length and modulating the level of a target gene mRNA, wherein the antisense oligonucleotide reduces the level of apolipoprotein B mRNA and wherein the target gene is selected from the group consisting of Lcat, Lip1, Lipc, Ppara, Pparg, Pcx, Apoa4, Apoc1, Apoc2, Apoc4, Mttp, Prkaa1, Prkaa2, Prkab1, Prkag1, Srebp-1, Scd2, Scd1, Acad1, Acadm, Acads, Acox1, Cpt1a, Cpt2, Crat, Elovl2, Elovl3, Acadsb, Fads2, Fasn, Facl2, Facl4, Abcd2, Dbi, Fabp1, Fabp2, Fabp7, Acat-1, Acca-1, Cyp7a1, Cyp7b1, Soat2, Ldlr, Hmgcs1, Hmgcs2, Car5a, Gck, Gck and G6 pc. In some aspects, the target gene mRNA is reduced, and this reduction occurs in a time-dependent manner or in a dose-dependent manner. Alternatively, the target gene mRNA is increased in a time-dependent manner or in a dose-dependent manner. In further aspects, the modulation of the target gene mRNA levels occurs in both a time- and dose-dependent manner.

Further provided are methods that result in a shift of a gene expression profile of an obese animal to that of a lean animal. Such methods comprise contacting an animal with an antisense oligonucleotide 15 to 30 nucleobases in length targeted to apolipoprotein B, resulting in the shift of a gene expression profile of an obese animal to that of a lean animal. In one aspect, the gene expression profile is a liver gene expression profile.

The invention also provides methods of reducing the risk of cardiovascular disease in an individual comprising the step of administering to an individual an amount of a compound of the invention sufficient to inhibit apolipoprotein B expression and modulate a gene expression profile. Risk factors for cardiovascular disease that are recognized by the Adult Treatment Panel III of the National Cholesterol Education Program include: previous coronary events, a family history of cardiovascular disease, elevated LDL-cholesterol, low HDL-cholesterol, elevated serum triglyceride, obesity, and physical inactivity, and metabolic syndrome.

The invention further provides methods of inhibiting the expression of apolipoprotein B and modulating a gene expression profile in cells or tissues comprising contacting said cells or tissues with a compound of the invention so that expression of apolipoprotein B is inhibited. Methods are also provided for treating an animal having a cardiovascular disease or condition comprising administering to said animal a therapeutically or prophylactically effective amount of a compound of the invention so that expression of apolipoprotein B is inhibited and gene expression profiles are altered. In various aspects, the condition is associated with abnormal lipid metabolism, the condition is associated with abnormal cholesterol metabolism, the condition is atherosclerosis, the condition is an abnormal metabolic condition, the abnormal metabolic condition is hyperlipidemia, the disease is diabetes, the diabetes is Type 2 diabetes, the condition is obesity, and/or the disease is cardiovascular disease.

The invention also provides methods of preventing or delaying the onset of a disease or condition associated with cardiovascular disease in an animal comprising administering to said animal a therapeutically or prophylactically effective amount of a compound of the invention. In one aspect, the animal is a human. In other aspects, the condition is an abnormal metabolic condition, the abnormal metabolic condition is hyperlipidemia, the disease is diabetes, the diabetes is Type 2 diabetes, the condition is obesity, the condition is atherosclerosis, the condition involves abnormal lipid metabolism, and/or the condition involves abnormal cholesterol metabolism.

Preferred methods of administration of the compounds or compositions of the invention to an animal are intravenously, subcutaneously, or orally. Administrations can be repeated.

Further provides are methods for altering a cellular pathway or metabolic process comprising contacting a cell with an antisense oligonucleotide that specifically hybridizes to and inhibits the expression of a nucleic acid molecule encoding apolipoprotein B. Cellular pathways and metabolic processes include apoptosis, angiogenesis, leptic secretion and T-cell co-stimulation. In some aspects, the antisense oligonucleotide comprises SEQ ID NO: 20. In one embodiment, apoptosis is induced in cancer cells, for example, breast cancer cells. In a further embodiment, angiogenesis, leptin secretion and T-cell co-stimulation are inhibited.

### DETAILED DESCRIPTION OF THE INVENTION

The present invention employs oligomeric compounds, particularly antisense oligonucleotides, for use in modulating the level of nucleic acid molecules encoding apolipoprotein B, ultimately resulting in the modulation of the mRNA levels of genes whose expression patterns are characteristic of an obese animal. Such modulation of gene expression patterns shifts a gene profile of an obese animal to that of a lean animal. This is accomplished by providing antisense compounds which specifically hybridize with one or more nucleic acids encoding apolipoprotein B.

As used herein, the terms “target nucleic acid” and “nucleic acid encoding apolipoprotein B” encompass DNA encoding apolipoprotein B, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds which specifically hybridize to it is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of apolipoprotein B. In the context of the present invention, “modulation” means either an increase (stimulation) or a decrease (inhibition) in the expression of a gene. In the context of the present invention, inhibition is the preferred form of modulation of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding apolipoprotein B. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes has a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding apolipoprotein B, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense and other compounds of the invention which hybridize to the target and inhibit expression of the target are identified through experimentation, and the sequences of these compounds are hereinbelow identified as preferred embodiments of the invention. The target sites to which these preferred sequences are complementary are hereinbelow referred to as “active sites” and are therefore preferred sites for targeting. Therefore another embodiment of the invention encompasses compounds which hybridize to these active sites.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

For use in kits and diagnostics, the antisense compounds of the present invention, either alone or in combination with other antisense compounds or therapeutics, can be used as tools in differential and/or combinatorial analyses to elucidate expression patterns of a portion or the entire complement of genes expressed within cells and tissues.

Expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett, 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (reviewed in (To, Comb. Chem. High Throughput Screen, 2000, 3, 235-41).

The specificity and sensitivity of antisense is also harnessed by those of skill in the art for therapeutic uses. Antisense oligonucleotides have been employed as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. Thus, this term includes oligonucleotides composed of naturally-occurring nucleobases, sugars and covalent internucleoside (backbone) linkages (RNA and DNA) as well as oligonucleotides having non-naturally-occurring portions which function similarly (oligonucleotide mimetics). Oligonucleotide mimetics are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 50 nucleobases (i.e. from about 8 to about 50 linked nucleosides). Particularly preferred antisense compounds are antisense oligonucleotides, even more preferably those comprising from about 12, about 14, about 20 to about 30 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression. In preferred embodiments, the antisense compound is non-catalytic oligonucleotide, i.e., is not dependent on a catalytic property of the oligonucleotide for its modulating activity. Antisense compounds of the invention can include double-stranded molecules wherein a first strand is stably hybridized to a second strand.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleotides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[CH₂)ₙCH₃)]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples hereinbelow.

A further preferred modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (—CH₂—)ₙ group bridging the 2′ oxygen atom and the 4′ carbon atom wherein n is 1 or 2. LNAs and preparation thereof are described in WO 98/39352 and WO 99/14226.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,750,692; 5,763,588; 6,005,096; and 5,681,941, each of which is herein incorporated by reference.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhoda-mines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhausen et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Chimeric antisense compounds can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

Both gapmer and hemimer compounds have also been referred to in the art as hybrids. In a gapmer that is 20 nucleotides in length, a gap or wing can be 1, 2, 3, 4, 5, 6, 7, 8, 9, 10, 11, 12, 13, 14, 15, 16, 17 or 18 nucleotides in length. In one embodiment, a 20-nucleotide gapmer is comprised of a gap 8 nucleotides in length, flanked on both the 5′ and 3′ sides by wings 6 nucleotides in length. In another embodiment, a 20-nucleotide gapmer is comprised of a gap 10 nucleotides in length, flanked on both the 5′ and 3′ sides by wings 5 nucleotides in length. In another embodiment, a 20-nucleotide gapmer is comprised of a gap 12 nucleotides in length flanked on both the 5′ and 3′ sides by wings 4 nucleotides in length. In a further embodiment, a 20-nucleotide gapmer is comprised of a gap 14 nucleotides in length flanked on both the 5′ and 3′ sides by wings 3 nucleotides in length. In another embodiment, a 20-nucleotide gapmer is comprised of a gap 16 nucleotides in length flanked on both the 5′ and 3′ sides by wings 2 nucleotides in length. In a further embodiment, a 20-nucleotide gapmer is comprised of a gap 18 nucleotides in length flanked on both the 5′ and 3′ ends by wings 1 nucleotide in length. Alternatively, the wings are of different lengths, for example, a 20-nucleotide gapmer may be comprised of a gap 10 nucleotides in length, flanked by a 6-nucleotide wing on one side (5′ or 3′) and a 4-nucleotide wing on the other side (5′ or 3′). In a hemimer, an “open end” chimeric antisense compound, 20 nucleotides in length, a gap segment, located at either the 5′ or 3′ terminus of the oligomeric compound, can be 1, 2, 3, 4, 5, 6, 7, 8, 9, 10, 11, 12, 13, 14, 15, 16, 17, 18 or 19 nucleotides in length. For example, a 20-nucleotide hemimer can have a gap segment of 10 nucleotides at the 5′ end and a second segment of 10 nucleotides at the 3′ end. Alternatively, a 20-nucleotide hemimer can have a gap segment of 10 nucleotides at the 3′ end and a second segment of 10 nucleotides at the 5′ end.

Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl) phosphate] derivatives according to the methods disclosed in WO 93/24510 to Gosselin et al., published Dec. 9, 1993 or in WO 94/26764 and U.S. Pat. No. 5,770,713 to Imbach et al. The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of apolipoprotein B is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation or tumor formation, for example.

The primers and probes disclosed herein are useful for research and diagnostics, because these compounds hybridize to nucleic acids encoding apolipoprotein B, enabling sandwich and other assays to easily be constructed to exploit this fact. Hybridization of the disclosed primers and probes with a nucleic acid encoding apolipoprotein B can be detected by means known in the art. Such means may include conjugation of an enzyme to the oligonucleotide, radiolabelling of the oligonucleotide or any other suitable detection means. Kits using such detection means for detecting the level of apolipoprotein B in a sample may also be prepared.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Preferred topical formulations include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA). Oligonucleotides of the invention may be encapsulated within liposomes or may form complexes thereto, in particular to cationic liposomes. Alternatively, oligonucleotides may be complexed to lipids, in particular to cationic lipids. Preferred fatty acids and esters include but are not limited arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof. Topical formulations are described in detail in U.S. patent application Ser. No. 09/315,298 filed on May 20, 1999 which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Preferred fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. Oligonucleotide complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for oligonucleotides and their preparation are described in detail in U.S. application Ser. Nos. 08/886,829 (filed Jul. 1, 1997), 09/108,673 (filed Jul. 1, 1998), 09/256,515 (filed Feb. 23, 1999), 09/082,624 (filed May 21, 1998) and 09/315,298 (filed May 20, 1999) each of which is incorporated herein by reference in their entirety.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

### Emulsions

The compositions of the present invention may be prepared and formulated as emulsions. Emulsions are typically heterogenous systems of one liquid dispersed in another in the form of droplets usually exceeding 0.1 μm in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### Liposomes

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis.

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome® (glyceryl dilaurate/cholesterol/polyoxyethylene-10-stearyl ether) and Novasome® II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al. S.T.P.Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1}, or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C₁₂15G, that contains a PEG moiety. Ilium et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. Nos. 5,540,935 (Miyazaki et al.) and 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

### Penetration Enhancers

In one embodiment, the present invention employs various penetration enhancers to effect the efficient delivery of nucleic acids, particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

### Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′ isothiocyano-stilbene-2,2′-disulfonic acid (Miyao et al., Antisense Res. Dev., 1995, 5, 115-121; Takakura et al., Antisense & Nucl. Acid Drug Dev., 1996, 6, 177-183).

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

### Pulsatile Delivery

The compounds of the present invention may also be administered by pulsatile delivery. “Pulsatile delivery” refers to a pharmaceutical formulations that delivers a first pulse of drug combined with a penetration enhancer and a second pulse of penetration enhancer to promote absorption of drug which is not absorbed upon release with the first pulse of penetration enhancer.

One embodiment of the present invention is a delayed release oral formulation for enhanced intestinal drug absorption, comprising:

(a) a first population of carrier particles comprising said drug and a penetration enhancer, wherein said drug and said penetration enhancer are released at a first location in the intestine; and

(b) a second population of carrier particles comprising a penetration enhancer and a delayed release coating or matrix, wherein the penetration enhancer is released at a second location in the intestine downstream from the first location, whereby absorption of the drug is enhanced when the drug reaches the second location.

Alternatively, the penetration enhancer in (a) and (b) is different.

This enhancement is obtained by encapsulating at least two populations of carrier particles. The first population of carrier particles comprises a biologically active substance and a penetration enhancer, and the second (and optionally additional) population of carrier particles comprises a penetration enhancer and a delayed release coating or matrix.

A “first pass effect” that applies to orally administered drugs is degradation due to the action of gastric acid and various digestive enzymes. One means of ameliorating first pass clearance effects is to increase the dose of administered drug, thereby compensating for proportion of drug lost to first pass clearance. Although this may be readily achieved with i.v. administration by, for example, simply providing more of the drug to an animal, other factors influence the bioavailability of drugs administered via non-parenteral means. For example, a drug may be enzymatically or chemically degraded in the alimentary canal or blood stream and/or may be impermeable or semipermeable to various mucosal membranes.

It is also contemplated that these pharmaceutical compositions are capable of enhancing absorption of biologically active substances when administered via the rectal, vaginal, nasal or pulmonary routes. It is also contemplated that release of the biologically active substance can be achieved in any part of the gastrointestinal tract.

Liquid pharmaceutical compositions of oligonucleotide can be prepared by combining the oligonucleotide with a suitable vehicle, for example sterile pyrogen free water, or saline solution. Other therapeutic compounds may optionally be included.

The present invention also contemplates the use of solid particulate compositions. Such compositions preferably comprise particles of oligonucleotide that are of respirable size. Such particles can be prepared by, for example, grinding dry oligonucleotide by conventional means, fore example with a mortar and pestle, and then passing the resulting powder composition through a 400 mesh screen to segregate large particles and agglomerates. A solid particulate composition comprised of an active oligonucleotide can optionally contain a dispersant which serves to facilitate the formation of an aerosol, for example lactose.

In accordance with the present invention, oligonucleotide compositions can be aerosolized. Aerosolization of liquid particles can be produced by any suitable means, such as with a nebulizer. See, for example, U.S. Pat. No. 4,501,729. Nebulizers are commercially available devices which transform solutions or suspensions into a therapeutic aerosol mist either by means of acceleration of a compressed gas, typically air or oxygen, through a narrow venturi orifice or by means of ultrasonic agitation. Suitable nebulizers include those sold by Blairex® under the name PAR1 LC PLUS, PARI DURA-NEB 2000, PARI-BABY Size, PARI PRONEB Compressor with LC PLUS, PARI WALKHALER Compressor/Nebulizer System, PAR1 LC PLUS Reusable Nebulizer, and PAR1 LC Jet+Nebulizer.

Exemplary formulations for use in nebulizers consist of an oligonucleotide in a liquid, such as sterile, pyragen free water, or saline solution, wherein the oligonucleotide comprises up to about 40% w/w of the formulation. Preferably, the oligonucleotide comprises less than 20% w/w. If desired, further additives such as preservatives (for example, methyl hydroxybenzoate) antioxidants, and flavoring agents can be added to the composition.

Solid particles comprising an oligonucleotide can also be aerosolized using any solid particulate medicament aerosol generator known in the art. Such aerosol generators produce respirable particles, as described above, and further produce reproducible metered dose per unit volume of aerosol. Suitable solid particulate aerosol generators include insufflators and metered dose inhalers. Metered dose inhalers are used in the art and are useful in the present invention.

Preferably, liquid or solid aerosols are produced at a rate of from about 10 to 150 liters per minute, more preferably from about 30 to 150 liters per minute, and most preferably about 60 liters per minute.

Enhanced bioavailability of biologically active substances is also achieved via the oral administration of the compositions and methods of the present invention. The term “bioavailability” refers to a measurement of what portion of an administered drug reaches the circulatory system when a non-parenteral mode of administration is used to introduce the drug into an animal.

Penetration enhancers include, but are not limited to, members of molecular classes such as surfactants, fatty acids, bile salts, chelating agents, and non-chelating non-surfactant molecules. (Lee et al., Critical Reviews in Therapeutic Drug Carrier Systems, 1991, p. 92). Carriers are inert molecules that may be included in the compositions of the present invention to interfere with processes that lead to reduction in the levels of bioavailable drug.

### Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). See, generally, The Merck Manual of Diagnosis and Therapy, 15th Ed. 1987, pp. 1206-1228, Berkow et al., eds., Rahway, N. J. When used with the compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N. J., pages 2499-2506 and 46-49, respectively). Other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models. In general, dosage is from 0.01 ug to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

The effects of treatments with therapeutic compositions can be assessed following collection of tissues or fluids from a patient or subject receiving said treatments. It is known in the art that a biopsy sample can be procured from certain tissues without resulting in detrimental effects to a patient or subject. In certain embodiments, a tissue and its constituent cells comprise, but are not limited to, blood (e.g., hematopoietic cells, such as human hematopoietic progenitor cells, human hematopoietic stem cells, CD34⁺cells CD4⁺cells), lymphocytes and other blood lineage cells, bone marrow, breast, cervix, colon, esophagus, lymph node, muscle, peripheral blood, oral mucosa and skin. In other embodiments, a fluid and its constituent cells comprise, but are not limited to, blood, urine, semen, synovial fluid, lymphatic fluid and cerebro-spinal fluid. Tissues or fluids procured from patients can be evaluated for expression levels of the target mRNA or protein. Additionally, the mRNA or protein expression levels of other genes known or suspected to be associated with the specific disease state, condition or phenotype can be assessed. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the disease or condition in the aforementioned tissues and fluids, collected from a patient or subject receiving treatment, by routine clinical methods known in the art. These biomarkers include but are not limited to: glucose, cholesterol, lipoproteins, triglycerides, free fatty acids and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

### Combination Therapy

The invention also provides methods of combination therapy, wherein one or more compounds of the invention and one or more other therapeutic/prophylactic compounds are administered treat a condition and/or disease state as described herein. In various aspects, the compound(s) of the invention and the therapeutic/prophylactic compound(s) are co-administered as a mixture or administered individually. In one aspect, the route of administration is the same for the compound(s) of the invention and the therapeutic/prophylactic compound(s), while in other aspects, the compound(s) of the invention and the therapeutic/prophylactic compound(s) are administered by a different routes. In one embodiment, the dosages of the compound(s) of the invention and the therapeutic/prophylactic compound(s) are amounts that are therapeutically or prophylactically effective for each compound when administered individually. Alternatively, the combined administration permits use of lower dosages than would be required to achieve a therapeutic or prophylactic effect if administered individually, and such methods are useful in decreasing one or more side effects of the reduced-dose compound.

In one aspect, a compound of the present invention and one or more other therapeutic/prophylactic compound(s) effective at treating a condition are administered wherein both compounds act through the same or different mechanisms. Therapeutic/prophylactic compound(s) include, but are not limited to, bile salt sequestering resins (e.g., cholestyramine, colestipol, and colesevelam hydrochloride), HMGCoA-redectase inhibitors (e.g., lovastatin, cerivastatin, prevastatin, atorvastatin, simvastatin, and fluvastatin), nicotinic acid, fibric acid derivatives (e.g., clofibrate, gemfibrozil, fenofibrate, bezafibrate, and ciprofibrate), probucol, neomycin, dextrothyroxine, plant-stanol esters, cholesterol absorption inhibitors (e.g., ezetimibe), implitapide, inhibitors of bile acid transporters (apical sodium-dependent bile acid transporters), regulators of hepatic CYP7a, estrogen replacement therapeutics (e.g., tamoxigen), and anti-inflammatories (e.g., glucocorticoids).

Accordingly, the invention further provides use of a compound of the invention and one or more other therapeutic/prophylactic compound(s) as described herein in the manufacture of a medicament for the treatment and/or prevention of a disease or condition as described herein.

### Targeted Delivery

In another aspect, methods are provided to target a compound of the invention to a specific tissue, organ or location in the body. Exemplary targets include liver, lung, kidney, heart, and atherosclerotic plaques within a blood vessel. Methods of targeting compounds are well known in the art.

In one embodiment, the compound is targeted by direct or local administration. For example, when targeting a blood vessel, the compound is administered directly to the relevant portion of the vessel from inside the lumen of the vessel, e.g., single balloon or double balloon catheter, or through the adventitia with material aiding slow release of the compound, e.g., a pluronic gel system as described by Simons et al., Nature 359: 67-70 (1992). Other slow release techniques for local delivery of the compound to a vessel include coating a stent with the compound. Methods of delivery of antisense compounds to a blood vessel are disclosed in U.S. Pat. No. 6,159,946, which is incorporated by reference in its entirety.

When targeting a particular tissue or organ, the compound may be administered in or around that tissue or organ. For example, U.S. Pat. No. 6,547,787, incorporated herein by reference in its entirety, discloses methods and devices for targeting therapeutic agents to the heart. In one aspect, administration occurs by direct injection or by injection into a blood vessel associated with the tissue or organ. For example, when targeting the liver, the compound may be administered by injection or infusion through the portal vein.

In another aspect, methods of targeting a compound are provided which include associating the compound with an agent that directs uptake of the compound by one or more cell types. Exemplary agents include lipids and lipid-based structures such as liposomes generally in combination with an organ- or tissue-specific targeting moiety such as, for example, an antibody, a cell surface receptor, a ligand for a cell surface receptor, a polysaccharide, a drug, a hormone, a hapten, a special lipid and a nucleic acid as described in U.S. Pat. No. 6,495,532, the disclosure of which is incorporated herein by reference in its entirety. U.S. Pat. No. 5,399,331, the disclosure of which is incorporated herein by reference in its entirety, describes the coupling of proteins to liposomes through use of a crosslinking agent having at least one maleimido group and an amine reactive function; U.S. Pat. Nos. 4,885,172, 5,059,421 and 5,171,578, the disclosures of which are incorporated herein by reference in their entirety, describe linking proteins to liposomes through use of the glycoprotein streptavidin and coating targeting liposomes with polysaccharides. Other lipid based targeting agents include, for example, micelle and crystalline products as described in U.S. Pat. No. 6,217,886, the disclosure of which is incorporated herein by reference in its entirety.

In another aspect, targeting agents include porous polymeric microspheres which are derived from copolymeric and homopolymeric polyesters containing hydrolyzable ester linkages which are biodegradable, as described in U.S. Pat. No. 4,818,542, the disclosure of which is incorporated herein by reference in its entirety. Typical polyesters include polyglycolic (PGA) and polylactic (PLA) acids, and copolymers of glycolide and L(-lactide) (PGL), which are particularly suited for the methods and compositions of the present invention in that they exhibit low human toxicity and are biodegradable. The particular polyester or other polymer, oligomer, or copolymer utilized as the microspheric polymer matrix is not critical and a variety of polymers may be utilized depending on desired porosity, consistency, shape and size distribution. Other biodegradable or bioerodable polymers or copolymers include, for example, gelatin, agar, starch, arabinogalactan, albumin, collagen, natural and synthetic materials or polymers, such as, poly(ε-caprolactone), poly(ε-caprolactone-CO-lactic acid), poly(ε-caprolactone-CO-glycolic acid), poly(β-hydroxy butyric acid), polyethylene oxide, polyethylene, poly(alkyl-2-cyanoacrylate), (e.g., methyl, ethyl, butyl), hydrogels such as poly(hydroxyethyl methacrylate), polyamides (e.g., polyacrylamide), poly(amino acids) (i.e., L-leucine, L-aspartic acid, β-methyl-L-aspartate, β-benzyl-L-aspartate, glutamic acid), poly(2-hydroxyethyl DL-aspartamide), poly(ester urea), poly(L-phenylalanine/ethylene glycol/1,6-diisocyanatohexane) and poly(methyl methacrylate). The exemplary natural and synthetic polymers suitable for targeted delivery are either readily available commercially or are obtainable by condensation polymerization reactions from the suitable monomers or, comonomers or oligomers.

In still another embodiment, U.S. Pat. No. 6,562,864, the disclosure of which is incorporated herein by reference in its entirety, describes catechins, including epi and other carbo-cationic isomers and derivatives thereof, which as monomers, dimers and higher multimers can form complexes with nucleophilic and cationic bioactive agents for use as delivery agents. Catechin multimers have a strong affinity for polar proteins, such as those residing in the vascular endothelium, and on cell/organelle membranes and are particularly useful for targeted delivery of bioactive agents to select sites in vivo. In treatment of vascular diseases and disorders, such as atherosclerosis and coronary artery disease, delivery agents include substituted catechin multimers, including amidated catechin multimers which are formed from reaction between catechin and nitrogen containing moities such as ammonia.

Other targeting strategies of the invention include ADEPT (antibody-directed enzyme prodrug therapy), GDEPT (gene-directed EPT) and VDEPT (virus-directed EPT) as described in U.S. Pat. No. 6,433,012, the disclosure of which is incorporated herein by reference in its entirety.

The present invention further provides medical devices and kits for targeted delivery, wherein the device is, for example, a syringe, stent, or catheter. Kits include a device for administering a compound and a container comprising a compound of the invention. In one aspect, the compound is preloaded into the device. In other embodiments, the kit provides instructions for methods of administering the compound and dosages. U.S. patents describing medical devices and kits for delivering antisense compounds include U.S. Pat. Nos. 6,368,356; 6,344,035; 6,344,028; 6,287,285; 6,200,304; 5,824,049; 5,749,915; 5,674,242; 5,670,161; 5,609,629; 5,593,974; and 5,470,307 (all incorporated herein by reference in their entirety).

The present invention further provides methods for shifting a gene expression profile of an animal from that of an obese animal to that of a lean animal. A “lean animal” is an animal on a standard diet that is not considered to have a hyperlipidemic condition. An “obese animal” is obese and/or consumes a high-fat diet, and exhibits one or more indicators of hyperlipidemia, for example, elevated serum LDL-cholesterol, lowered serum HDL-cholesterol, or elevated serum triglycerides. Expression profiles are identified by the comparison of mRNA levels in a lean animal (“lean animal profile” or “lean profile”) with mRNA levels of selected genes in a high-fat fed or obese animal (“obese animal profile” or “obese profile”). A lean animal gene expression profile is characterized by the reduction of mRNA levels of about 5-10 genes, selected from the group consisting of Lip1, Ppara, Pparg, Pcx, Apoa4, Apoc1, Apoc2, Apoc4, Mttp, Srebp-1, Scd1, Acad1, Acadm, Acads, Acox1, Cpt1a, Cpt2, Crat, Elovl2, Elovl3, Acadsb, Fads2, Facl2, Dbi, Fabp1, Fabp2, Acat-1, Acca-1, Hmgcs1, Hmgcs2, Gck, and G6 pc. In addition, a lean animal gene expression profile is characterized by the increase of mRNA levels of at least 2 genes selected from the group consisting of Prkaa2, Prkab1, Scd2, and Soat2. Methods for shifting a gene expression profile from that of an obese animal to that of a lean animal include contacting an animal with an antisense oligonucleotide targeted to apolipoprotein B, which results in a gene expression profile characteristic of a lean animal. Also provided are methods for differentiating a lean animal profile from a high-fat, apolipoprotein B oligonucleotide-treated animal profile. Such differentiating genes are Prkag1, Facl4, Fabp7, and Cyp7b1, 2 or more of which are lowered in lean animals, but are raised in high-fat fed, apolipoprotein B oligonucleotide-treated animals. Additional differentiating genes are Lip1, Lipc, Scd1, Cpt1a, Fasn, Abcd2, Dbi, Cyp7a1, Ldlr, Hmgcs1, and Car5a, 2 or more of which are raised in lean animals, but are lowered in high-fat fed, apolipoprotien B oligonucleotide-treated animals.

While the present invention has been described with specificity in accordance with certain embodiments, the following examples serve only to illustrate the invention and are not intended to limit the same. Each of the references, GENBANK® accession numbers, and the like recited in the present application is incorporated herein by reference in its entirety.

### EXAMPLES

### Example 1

### Nucleoside Phosphoramidites for Oligonucleotide Synthesis Deoxy and 2′-alkoxy amidites

2′-Deoxy and 2′-methoxy beta-cyanoethyldiisopropyl phosphoramidites were purchased from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, the standard cycle for unmodified oligonucleotides was utilized, except the wait step after pulse delivery of tetrazole and base was increased to 360 seconds.

Oligonucleotides containing 5-methyl-2′-deoxycytidine (5-Me-C) nucleotides were synthesized according to published methods (Sanghvi, et. al., Nucleic Acids Research, 1993, 21, 3197-3203] using commercially available phosphoramidites (Glen Research, Sterling Va. or ChemGenes, Needham Mass.).

### 2′-Fluoro amidites

### 2′-Fluorodeoxyadenosine amidites

2′-fluoro oligonucleotides were synthesized as described previously (Kawasaki, et. al., J. Med. Chem., 1993, 36, 831-841] and U.S. Pat. No. 5,670,633, herein incorporated by reference. Briefly, the protected nucleoside N6-benzoyl-2′-deoxy-2′-fluoroadenosine was synthesized utilizing commercially available 9-beta-D-arabinofuranosyladenine as starting material and by modifying literature procedures whereby the 2′-alpha-fluoro atom is introduced by a S_{N}²-displacement of a 2′-beta-trityl group. Thus N6-benzoyl-9-beta-D-arabinofuranosyladenine was selectively protected in moderate yield as the 3′,5′-ditetrahydropyranyl (THP) intermediate. Deprotection of the THP and N6-benzoyl groups was accomplished using standard methodologies and standard methods were used to obtain the 5′-dimethoxytrityl-(DMT) and 5′-DMT-3′-phosphoramidite intermediates.

### 2′-Fluorodeoxyguanosine

The synthesis of 2′-deoxy-2′-fluoroguanosine was accomplished using tetraisopropyldisiloxanyl (TPDS) protected 9-beta-D-arabinofuranosylguanine as starting material, and conversion to the intermediate diisobutyrylarabinofuranosylguanosine. Deprotection of the TPDS group was followed by protection of the hydroxyl group with THP to give diisobutyryl di-THP protected arabinofuranosylguanine. Selective O-deacylation and triflation was followed by treatment of the crude product with fluoride, then deprotection of the THP groups.

Standard methodologies were used to obtain the 5′-DMT- and 5′-DMT-3′-phosphoramidites.

### 2′-Fluorouridine

Synthesis of 2′-deoxy-2′-fluorouridine was accomplished by the modification of a literature procedure in which 2,2′-anhydro-1-beta-D-arabinofuranosyluracil was treated with 70% hydrogen fluoride-pyridine. Standard procedures were used to obtain the 5′-DMT and 5′-DMT-3′ phosphoramidites.

### 2′-Fluorodeoxycytidine

2′-deoxy-T-fluorocytidine was synthesized via amination of 2′-deoxy-2′-fluorouridine, followed by selective protection to give N4-benzoyl-2′-deoxy-2′-fluorocytidine. Standard procedures were used to obtain the 5′-DMT and 5′-DMT-3′ phosphoramidites.

### 2′-O-(2-Methoxyethyl) modified amidites

2′-O-Methoxyethyl-substituted nucleoside amidites are prepared as follows, or alternatively, as per the methods of Martin, P., Helvetica Chimica Acta, 1995, 78, 486-504.

### 2,2′-Anhydro[1-(beta-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) were added to DMF (300 mL). The mixture was heated to reflux, with stirring, allowing the evolved carbon dioxide gas to be released in a controlled manner. After 1 hour, the slightly darkened solution was concentrated under reduced pressure. The resulting syrup was poured into diethylether (2.5 L), with stirring. The product formed a gum. The ether was decanted and the residue was dissolved in a minimum amount of methanol (ca. 400 mL). The solution was poured into fresh ether (2.5 L) to yield a stiff gum. The ether was decanted and the gum was dried in a vacuum oven (60° C. at 1 mm Hg for 24 h) to give a solid that was crushed to a light tan powder (57 g, 85% crude yield). The NMR spectrum was consistent with the structure, contaminated with phenol as its sodium salt (ca. 5%). The material was used as is for further reactions (or it can be purified further by column chromatography using a gradient of methanol in ethyl acetate (10-25%) to give a white solid, mp 222-4° C.).

### 2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) were added to a 2 L stainless steel pressure vessel and placed in a pre-heated oil bath at 160° C. After heating for 48 hours at 155-160° C., the vessel was opened and the solution evaporated to dryness and triturated with MeOH (200 mL). The residue was suspended in hot acetone (1 L). The insoluble salts were filtered, washed with acetone (150 mL) and the filtrate evaporated. The residue (280 g) was dissolved in CH₃CN (600 mL) and evaporated. A silica gel column (3 kg) was packed in CH₂Cl₂/acetone/MeOH (20:5:3) containing 0.5% Et₃NH. The residue was dissolved in CH₂Cl₂ (250 mL) and adsorbed onto silica (150 g) prior to loading onto the column. The product was eluted with the packing solvent to give 160 g (63%) of product. Additional material was obtained by reworking impure fractions.

### 2′-O-Methoxyethyl-5′-O-dimethoxytrityl-5-methyluridine

2′-O-Methoxyethyl-5-methyluridine (160 g, 0.506 M) was co-evaporated with pyridine (250 mL) and the dried residue dissolved in pyridine (1.3 L). A first aliquot of dimethoxytrityl chloride (94.3 g, 0.278 M) was added and the mixture stirred at room temperature for one hour. A second aliquot of dimethoxytrityl chloride (94.3 g, 0.278 M) was added and the reaction stirred for an additional one hour. Methanol (170 mL) was then added to stop the reaction. HPLC showed the presence of approximately 70% product. The solvent was evaporated and triturated with CH₃CN (200 mL). The residue was dissolved in CHCl₃ (1.5 L) and extracted with 2×500 mL of saturated NaHCO₃ and 2×500 mL of saturated NaCl. The organic phase was dried over Na₂SO₄, filtered and evaporated. 275 g of residue was obtained. The residue was purified on a 3.5 kg silica gel column, packed and eluted with EtOAc/hexane/acetone (5:5:1) containing 0.5% Et₃NH. The pure fractions were evaporated to give 164 g of product. Approximately 20 g additional was obtained from the impure fractions to give a total yield of 183 g (57%).

### 3′O-Acetyl-2′-O-methoxyethyl-5′-O-dimethoxytrityl-5-methyluridine

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) were combined and stirred at room temperature for 24 hours. The reaction was monitored by TLC by first quenching the TLC sample with the addition of MeOH. Upon completion of the reaction, as judged by TLC, MeOH (50 mL) was added and the mixture evaporated at 35° C. The residue was dissolved in CHCl₃ (800 mL) and extracted with 2×200 mL of saturated sodium bicarbonate and 2×200 mL of saturated NaCl. The water layers were back extracted with 200 mL of CHCl₃. The combined organics were dried with sodium sulfate and evaporated to give 122 g of residue (approx. 90% product). The residue was purified on a 3.5 kg silica gel column and eluted using EtOAc/hexane(4:1). Pure product fractions were evaporated to yield 96 g (84%). An additional 1.5 g was recovered from later fractions.

### 3′-O-Acetyl-2′-O-methoxyethyl-5′-O-dimethoxytrityl-5-methyl-4-triazoleuridine

A first solution was prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) was added to a solution of triazole (90 g, 1.3 M) in CH₃CN (1 L), cooled to −5° C. and stirred for 0.5 h using an overhead stirrer. POCl₃ was added dropwise, over a 30 minute period, to the stirred solution maintained at 0-10° C., and the resulting mixture stirred for an additional 2 hours. The first solution was added dropwise, over a 45 minute period, to the latter solution. The resulting reaction mixture was stored overnight in a cold room. Salts were filtered from the reaction mixture and the solution was evaporated. The residue was dissolved in EtOAc (1 L) and the insoluble solids were removed by filtration. The filtrate was washed with 1×300 mL of NaHCO₃ and 2×300 mL of saturated NaCl, dried over sodium sulfate and evaporated. The residue was triturated with EtOAc to give the title compound.

### 2′-O-Methoxyethyl-5-O-dimethoxytrityl-5-methylcytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) was stirred at room temperature for 2 hours. The dioxane solution was evaporated and the residue azeotroped with MeOH (2×200 mL). The residue was dissolved in MeOH (300 mL) and transferred to a 2 liter stainless steel pressure vessel. MeOH (400 mL) saturated with NH₃ gas was added and the vessel heated to 100° C. for 2 hours (TLC showed complete conversion). The vessel contents were evaporated to dryness and the residue was dissolved in EtOAc (500 mL) and washed once with saturated NaCl (200 mL). The organics were dried over sodium sulfate and the solvent was evaporated to give 85 g (95%) of the title compound.

### N4-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) was dissolved in DMF (800 mL) and benzoic anhydride (37.2 g, 0.165 M) was added with stirring. After stirring for 3 hours, TLC showed the reaction to be approximately 95% complete. The solvent was evaporated and the residue azeotroped with MeOH (200 mL). The residue was dissolved in CHCl₃ (700 mL) and extracted with saturated NaHCO₃ (2×300 mL) and saturated NaCl (2×300 mL), dried over MgSO₄ and evaporated to give a residue (96 g). The residue was chromatographed on a 1.5 kg silica column using EtOAc/hexane (1:1) containing 0.5% Et₃NH as the eluting solvent. The pure product fractions were evaporated to give 90 g (90%) of the title compound.

### N4-Benzoyl-2′-O-methoxyethyl-5′-O-dimethoxytrityl-5-methylcytidine-3′-amidite

N4-Benzoyl-2′-O-methoxyethyl-5′-O-dimethoxytrityl-5-methylcytidine (74 g, 0.10 M) was dissolved in CH₂Cl₂ (1 L). Tetrazole diisopropylamine (7.1 g) and 2-cyanoethoxy-tetra(isopropyl)-phosphite (40.5 mL, 0.123 M) were added with stirring, under a nitrogen atmosphere. The resulting mixture was stirred for 20 hours at room temperature (TLC showed the reaction to be 95% complete). The reaction mixture was extracted with saturated NaHCO₃ (1×300 mL) and saturated NaCl (3×300 mL). The aqueous washes were back-extracted with CH₂Cl₂ (300 mL), and the extracts were combined, dried over MgSO₄ and concentrated. The residue obtained was chromatographed on a 1.5 kg silica column using EtOAc/hexane (3:1) as the eluting solvent. The pure fractions were combined to give 90.6 g (87%) of the title compound.

### 2′-O-(Aminooxyethyl) nucleoside amidites and 2′-O-(dimethylaminooxyethyl) nucleoside amidites

### 2′-(Dimethylaminooxyethoxy) nucleoside amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

### 5′-O-tert-Butyldiphenylsilyl-O²-2′-anhydro-5-methyluridine

O²-2′-anhydro-5-methyluridine (Pro. Bio. Sint., Varese, Italy, 100.0 g, 0.416 mmol), dimethylaminopyridine (0.66 g, 0.013 eq, 0.0054 mmol) were dissolved in dry pyridine (500 ml) at ambient temperature under an argon atmosphere and with mechanical stirring. tert-Butyldiphenylchlorosilane (125.8 g, 119.0 mL, 1.1 eq, 0.458 mmol) was added in one portion. The reaction was stirred for 16 h at ambient temperature. TLC(Rf 0.22, ethyl acetate) indicated a complete reaction. The solution was concentrated under reduced pressure to a thick oil. This was partitioned between dichloromethane (1 L) and saturated sodium bicarbonate (2×1 L) and brine (1 L). The organic layer was dried over sodium sulfate and concentrated under reduced pressure to a thick oil. The oil was dissolved in a 1:1 mixture of ethyl acetate and ethyl ether (600 mL) and the solution was cooled to −10° C. The resulting crystalline product was collected by filtration, washed with ethyl ether (3×200 mL) and dried (40° C., 1 mm Hg, 24 h) to 149 g (74.8%) of white solid. TLC and NMR were consistent with pure product.

### 5′-O-tert-Butyldiphenylsilyl-2′-O-(2-hydroxyethyl)-5-methyluridine

In a 2 L stainless steel, unstirred pressure reactor was added borane in tetrahydrofuran (1.0 M, 2.0 eq, 622 mL). In the fume hood and with manual stirring, ethylene glycol (350 mL, excess) was added cautiously at first until the evolution of hydrogen gas subsided. 5′-O-tert-Butyldiphenylsilyl-O²-2′-anhydro-5-methyluridine (149 g, 0.311 mol) and sodium bicarbonate (0.074 g, 0.003 eq) were added with manual stirring. The reactor was sealed and heated in an oil bath until an internal temperature of 160° C. was reached and then maintained for 16 h (pressure<100 prig). The reaction vessel was cooled to ambient and opened. TLC(Rf 0.67 for desired product and Rf 0.82 for ara-T side product, ethyl acetate) indicated about 70% conversion to the product. In order to avoid additional side product formation, the reaction was stopped, concentrated under reduced pressure (10 to 1 mm Hg) in a warm water bath (40-100° C.) with the more extreme conditions used to remove the ethylene glycol. Alternatively, once the low boiling solvent is gone, the remaining solution can be partitioned between ethyl acetate and water; the product will be in the organic phase. The residue was purified by column chromatography (2 kg silica gel, ethyl acetate-hexanes gradient 1:1 to 4:1). The appropriate fractions were combined, stripped and dried to product as a white crisp foam (84 g, 50%), contaminated starting material (17.4 g) and pure reusable starting material 20 g. The yield based on starting material less pure recovered starting material was 58%. TLC and NMR were consistent with 99% pure product.

### 2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) was mixed with triphenylphosphine (11.63 g, 44.36 mmol) and N-hydroxyphthalimide (7.24 g, 44.36 mmol). It was then dried over P₂O₅ under high vacuum for two days at 40° C. The reaction mixture was flushed with argon and dry THF (369.8 mL, Aldrich, sure seal bottle) was added to get a clear solution. Diethyl-azodicarboxylate (6.98 mL, 44.36 mmol) was added dropwise to the reaction mixture. The rate of addition is maintained such that resulting deep red coloration is just discharged before adding the next drop. After the addition was complete, the reaction was stirred for 4 hrs. By that time TLC showed the completion of the reaction (ethylacetate:hexane, 60:40). The solvent was evaporated in vacuum. Residue obtained was placed on a flash column and eluted with ethyl acetate:hexane (60:40), to get 2′4)4[2-phthalimidoxy)ethyl]-5′-t-butyldiphenylsilyl-5-methyluridine as white foam (21.819 g, 86%).

### 5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) was dissolved in dry CH₂Cl₂ (4.5 mL) and methylhydrazine (300 mL, 4.64 mmol) was added dropwise at −10° C. to 0° C. After 1 h the mixture was filtered, the filtrate was washed with ice cold CH₂Cl₂ and the combined organic phase was washed with water, brine and dried over anhydrous Na₂SO₄. The solution was concentrated to get 2′-O-(aminooxyethyl) thymidine, which was then dissolved in MeOH (67.5 mL). To this formaldehyde (20% aqueous solution, w/w, 1.1 eq.) was added and the resulting mixture was stirred for 1 h. Solvent was removed under vacuum; residue chromatographed to get 5′-O-tert-butyldiphenylsilyl-2′-O-[(2-formadoximinooxy) ethyl]-5-methyluridine as white foam (1.95 g, 78%).

### 5′-O-tert-Butyldiphenylsilyl-2′-O—[N,N-dimethylaminooxyethyl]-5-methyluridine

5′-O-tert-butyldiphenylsilyl-2′-O-[(2-formadoximinooxy)ethyl]-5-methyluridine (1.77 g, 3.12 mmol) was dissolved in a solution of 1M pyridinium p-toluenesulfonate (PPTS) in dry MeOH (30.6 mL). Sodium cyanoborohydride (0.39 g, 6.13 mmol) was added to this solution at 10° C. under inert atmosphere. The reaction mixture was stirred for 10 minutes at 10° C. After that the reaction vessel was removed from the ice bath and stirred at room temperature for 2 h, the reaction monitored by TLC (5% MeOH in CH₂Cl₂). Aqueous NaHCO₃ solution (5%, 10 mL) was added and extracted with ethyl acetate (2×20 mL). Ethyl acetate phase was dried over anhydrous Na₂SO₄, evaporated to dryness. Residue was dissolved in a solution of 1M PPTS in MeOH (30.6 mL). Formaldehyde (20% w/w, 30 mL, 3.37 mmol) was added and the reaction mixture was stirred at room temperature for 10 minutes. Reaction mixture cooled to 10° C. in an ice bath, sodium cyanoborohydride (0.39 g, 6.13 mmol) was added and reaction mixture stirred at 10° C. for 10 minutes. After 10 minutes, the reaction mixture was removed from the ice bath and stirred at room temperature for 2 hrs. To the reaction mixture 5% NaHCO₃ (25 mL) solution was added and extracted with ethyl acetate (2×25 mL). Ethyl acetate layer was dried over anhydrous Na₂SO₄ and evaporated to dryness. The residue obtained was purified by flash column chromatography and eluted with 5% MeOH in CH₂Cl₂ to get 5′-O-tert-butyldiphenylsilyl-2′-[N,N-dimethylaminooxyethyl]-5-methyluridine as a white foam (14.6 g, 80%).

### 2′-O-(dimethylaminooxyethyl)-5-methyluridine

Triethylamine trihydrofluoride (3.91 mL, 24.0 mmol) was dissolved in dry THF and triethylamine (1.67 mL, 12 mmol, dry, kept over KOH). This mixture of triethylamine-2HF was then added to 5′-O-tert-butyldiphenylsilyl-2′-O—[N,N-dimethylaminooxyethyl]-5-methyluridine (1.40 g, 2.4 mmol) and stirred at room temperature for 24 hrs. Reaction was monitored by TLC (5% MeOH in CH₂Cl₂). Solvent was removed under vacuum and the residue placed on a flash column and eluted with 10% MeOH in CH₂Cl₂ to get 2′-O-(dimethylaminooxyethyl)-5-methyluridine (766 mg, 92.5%).

### 5′-O-DMT-2′-O-(dimethylaminooxyethyl)-5-methyluridine

2′-O-(dimethylaminooxyethyl)-5-methyluridine (750 mg, 2.17 mmol) was dried over P₂O₅ under high vacuum overnight at 40° C. It was then co-evaporated with anhydrous pyridine (20 mL). The residue obtained was dissolved in pyridine (11 mL) under argon atmosphere. 4-dimethylaminopyridine (26.5 mg, 2.60 mmol), 4,4′-dimethoxytrityl chloride (880 mg, 2.60 mmol) was added to the mixture and the reaction mixture was stirred at room temperature until all of the starting material disappeared. Pyridine was removed under vacuum and the residue chromatographed and eluted with 10% MeOH in CH₂Cl_{2 (containing a few drops of pyridine) to get} 5′-O-DMT-2′-O-(dimethylamino-oxyethyl)-5-methyluridine (1.13 g, 80%).

### 5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) was co-evaporated with toluene (20 mL). To the residue N,N-diisopropylamine tetrazonide (0.29 g, 1.67 mmol) was added and dried over P₂O₅ under high vacuum overnight at 40° C. Then the reaction mixture was dissolved in anhydrous acetonitrile (8.4 mL) and 2-cyanoethyl-N,N,N¹,N₁-tetraisopropylphosphoramidite (2.12 mL, 6.08 mmol) was added. The reaction mixture was stirred at ambient temperature for 4 hrs under inert atmosphere. The progress of the reaction was monitored by TLC (hexane:ethyl acetate 1:1). The solvent was evaporated, then the residue was dissolved in ethyl acetate (70 mL) and washed with 5% aqueous NaHCO₃ (40 mL). Ethyl acetate layer was dried over anhydrous Na₂SO₄ and concentrated. Residue obtained was chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam (1.04 g, 74.9%).

### 2′-(Aminooxyethoxy) nucleoside amidites

2′-(Aminooxyethoxy) nucleoside amidites [also known in the art as 2′-O-(aminooxyethyl) nucleoside amidites] are prepared as described in the following paragraphs. Adenosine, cytidine and thymidine nucleoside amidites are prepared similarly.

### N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl) diaminopurine riboside along with a minor amount of the 3′-O-isomer. 2′-O-(2-ethylacetyl) diaminopurine riboside may be resolved and converted to 2′-O-(2-ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-β-diphenylcarbamoyl-2′-O-(2-hydroxyethyl)-5′-O-(4,4′-dimethoxytrityl)guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-([2-phthalmidoxy]ethyl)-5′-O-(4,4′-dimethoxytrityl)guanosine-3′-[(2-cyanoethyl)-N,N-diisopropylphosphoramidite].

### 2′-dimethylaminoethoxyethoxy (2′-DMAEOE) nucleoside amidites

2′-dimethylaminoethoxyethoxy nucleoside amidites (also known in the art as 2′-O-dimethyl-aminoethoxyethyl, i.e., 2′-O—CH₂—O—CH₂—N(CH₂)₂, or 2′-DMAEOE nucleoside amidites) are prepared as follows. Other nucleoside amidites are prepared similarly.

### 2′-O-[2(2-N,N-dimethylaminoethoxy)ethyl]-5-methyl uridine

2[2-(Dimethylamino)ethoxy]ethanol (Aldrich, 6.66 g, 50 mmol) is slowly added to a solution of borane in tetrahydrofuran (1 M, 10 mL, 10 mmol) with stirring in a 100 mL bomb. Hydrogen gas evolves as the solid dissolves. O²-,2′-anhydro-5-methyluridine (1.2 g, 5 mmol), and sodium bicarbonate (2.5 mg) are added and the bomb is sealed, placed in an oil bath and heated to 155° C. for 26 hours. The bomb is cooled to room temperature and opened. The crude solution is concentrated and the residue partitioned between water (200 mL) and hexanes (200 mL). The excess phenol is extracted into the hexane layer. The aqueous layer is extracted with ethyl acetate (3×200 mL) and the combined organic layers are washed once with water, dried over anhydrous sodium sulfate and concentrated. The residue is columned on silica gel using methanol/methylene chloride 1:20 (which has 2% triethylamine) as the eluent. As the column fractions are concentrated a colorless solid forms which is collected to give the title compound as a white solid.

### 5′-O-dimethoxytrityl-2′-O-[2(2-N,N-dimethylaminoethoxy)ethyl)]′-5-methyl uridine

To 0.5 g (1.3 mmol) of 2′-O—[2(2-N,N-dimethylaminoethoxy)ethyl)]-5-methyl uridine in anhydrous pyridine (8 mL), triethylamine (0.36 mL) and dimethoxytrityl chloride (DMT-Cl, 0.87 g, 2 eq.) are added and stirred for 1 hour. The reaction mixture is poured into water (200 mL) and extracted with CH₂Cl₂ (2×200 mL). The combined CH₂Cl₂ layers are washed with saturated NaHCO₃ solution, followed by saturated NaCl solution and dried over anhydrous sodium sulfate. Evaporation of the solvent followed by silica gel chromatography using MeOH:CH₂Cl₂:Et₃N (20:1, v/v, with 1% triethylamine) gives the title compound.

### 5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxy-N,N-diisopropyl phosphoramidite (1.1 mL, 2 eq.) are added to a solution of 5′-O-dimethoxytrityl-2′-O-12(2-N,N-dimethylaminoethoxy)ethyl)]-5-methyluridine (2.17 g, 3 mmol) dissolved in CH₂Cl₂ (20 mL) under an atmosphere of argon. The reaction mixture is stirred overnight and the solvent evaporated. The resulting residue is purified by silica gel flash column chromatography with ethyl acetate as the eluent to give the title compound.

### Example 2

### Oligonucleotide synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle was replaced by 0.2 M solution of 3H-1,2-benzodithiole-3-one 1,1-dioxide in acetonitrile for the stepwise thiation of the phosphite linkages. The thiation wait step was increased to 68 sec and was followed by the capping step. After cleavage from the CPG column and deblocking in concentrated ammonium hydroxide at 55° C. (18 h), the oligonucleotides were purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

### Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

### PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, and 5,719,262, herein incorporated by reference.

### Example 5

### Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]—[2′-O-Me] Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 s repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample was again lyophilized to dryness. The pellet is resuspended in 1M TBAF in THF for 24 hrs at room temperature to deprotect the 2′ positions. The reaction is then quenched with 1M TEAA and the sample is then reduced to ½ volume by rotovac before being desalted on a G25 size exclusion column. The oligo recovered is then analyzed spectrophotometrically for yield and for purity by capillary electrophoresis and by mass spectrometry.

### [2′-O-(2-Methoxyethyl)]-[2′-deoxy]-[2′-O-(Methoxyethyl)] Chimeric Phosphorothioate Oligonucleotides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiester]—[2′-deoxy Phosphorothioate]—[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleo-tides/oligonucleosides are synthesized according to U.S. Pat. No. 5,623,065, herein incorporated by reference.

### Example 6

### Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides were analyzed by polyacrylamide gel electrophoresis on denaturing gels and judged to be at least 85% full length material. The relative amounts of phosphorothioate and phosphodiester linkages obtained in synthesis were periodically checked by ³¹P nuclear magnetic resonance spectroscopy, and for some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

### Oligonucleotide Synthesis—96 Well Plate Format

Oligonucleotides were synthesized via solid phase P(III) phosphoramidite chemistry on an automated synthesizer capable of assembling 96 sequences simultaneously in a standard 96 well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites were purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

### Oligonucleotide Analysis—96 Well Plate Format

The concentration of oligonucleotide in each well was assessed by dilution of samples and UV absorption spectroscopy. The full-length integrity of the individual products was evaluated by capillary electrophoresis (CE) in either the 96 well format (BECKMAN P/ACE® MDQ) or, for individually prepared samples, on a commercial CE apparatus (e.g., BECKMAN P/ACE® 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

### Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, Ribonuclease protection assays, or real-time PCR.

HepG2 cells:

The human hepatoblastoma cell line HepG2 was obtained from the American Type Culture Collection (Manassas, Va.). HepG2 cells were routinely cultured in Eagle's MEM supplemented with 10% fetal bovine serum, non-essential amino acids, and 1 mM sodium pyruvate (Invitrogen Life Technologies, Carlsbad, Calif.). Cells were routinely passaged by trypsinization and dilution when they reached 90% confluence. Cells were seeded into 96-well plates (Falcon-Primaria #3872, BD Biosciences, Bedford, Mass.) at a density of approximately 7000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

AML12 cells:

The AML12 (alpha mouse liver 12) cell line was established from hepatocytes from a mouse (CD1 strain, line MT42) transgenic for human TGF alpha. Cells are cultured in a 1:1 mixture of Dulbecco's modified Eagle's medium and Ham's F12 medium with 0.005 mg/ml insulin, 0.005 mg/ml transferrin, 5 ng/ml selenium, and 40 ng/ml dexamethasone, and 90%:10% fetal bovine serum (medium and additives from Invitrogen Life Technologies, Carlsbad Calif. and Sigma-Aldrich, St. Louis, Mo.). For subculturing, spent medium is removed and fresh media of 0.25% trypsin, 0.03% EDTA solution is added. Fresh trypsin solution (1 to 2 ml) is added and the culture is left to sit at room temperature until the cells detach. Cells were routinely passaged by trypsinization and dilution when they reached approximately 90% confluence. Cells were seeded into 96-well plates (Falcon-Primaria #3872, BD Biosciences, Bedford, Mass.) at a density of approximately 7000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

### Primary Mouse Hepatocytes:

Primary mouse hepatocytes were prepared from CD-1 mice purchased from Charles River Labs (Wilmington, Mass.) and were routinely cultured in Hepatoyte Attachment Media (Invitrogen Life Technologies, Carlsbad, Calif.) supplemented with 10% Fetal Bovine Serum (Invitrogen Life Technologies, Carlsbad, Calif.), 250 nM dexamethasone (Sigma), and 10 nM bovine insulin (both from Sigma-Aldrich, St. Louis, Mo.). Cells were seeded into 96-well plates (Falcon-Primaria #3872, BD Biosciences, Bedford, Mass.) at a density of approximately 10,000 cells/well for use in antisense oligonucleotide transfection experiments. For Northern blotting or other analyses, cells are plated onto 100 mm or other standard tissue culture plates coated with rat tail collagen (200 ug/mL) (BD Biosciences, Bedford, Mass.) and treated similarly using appropriate volumes of medium and oligonucleotide.

Hep3B cells:

The human hepatocellular carcinoma cell line Hep3B was obtained from the American Type Culture Collection (Manassas, Va.). Hep3B cells were routinely cultured in Dulbeccos's MEM high glucose supplemented with 10% fetal bovine serum, L-glutamine and pyridoxine hydrochloride (Invitrogen Life Technologies, Carlsbad, Calif.). Cells were routinely passaged by trypsinization and dilution when they reached approximately 90% confluence. Cells were seeded into 24-well plates (Falcon-Primaria #3846, BD Biosciences, Bedford, Mass.) at a density of approximately 50,000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

### Hela Cells:

The human epitheloid carcinoma cell line HeLa was obtained from the American Tissue Type Culture Collection (Manassas, Va.). HeLa cells were routinely cultured in DMEM, high glucose (Invitrogen Corporation, Carlsbad, Calif.) supplemented with 10% fetal bovine serum (Invitrogen Corporation, Carlsbad, Calif.). Cells were routinely passaged by trypsinization and dilution when they reached approximately 90% confluence. Cells were seeded onto 96-well plates (Falcon-Primaria #3872, BD Biosciences, Bedford, Mass.) at a density of approximately 5,000 cells/well for use in antisense oligonucleotide transfection experiments. Alternatively, cells were seeded into 24-well plates (Falcon-Primaria #3846, BD Biosciences, Bedford, Mass.) at a density of approximately 50,000 cells/well for use in RT-PCR analysis. For Northern blotting or other analyses, cells may be seeded onto 100 mm or other standard tissue culture plates and treated similarly, using appropriate volumes of medium and oligonucleotide.

### Human Mammary Epithelial Cells:

Normal human mammary epithelial cells (HMECs) were obtained from the American Type Culture Collection (Manassas Va.). HMECs were routinely cultured in DMEM low glucose supplemented with 10% fetal bovine serum (Invitrogen Corporation, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 7000 cells/well for use in antisense oligonucleotide transfection experiments. For Northern blotting or other analyses, cells may be seeded onto 100 mm or other standard tissue culture plates and treated similarly, using appropriate volumes of medium and oligonucleotide.

Treatment with Antisense Compounds:

When cells reached 65-75% confluency, they were treated with oligonucleotide. Oligonucleotide was mixed with LIPOFECTIN® Invitrogen Life Technologies, Carlsbad, Calif.) in OPTI-MEM® 1 reduced serum medium (Invitrogen Life Technologies, Carlsbad, Calif.) to achieve the desired concentration of oligonucleotide and a LIPOFECTIN® concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEM® 1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is ISIS 13920 (TCCGTCATCGCTCCTCAGGG, SEQ ID NO: 1; targeted to human H-ras), a chimeric oligonucleotide having a 9 nucleotide gap segment composed of 2′-deoxynucleotides, which is flanked on the 5′ side and 3′ sides by 3 nucleotide and 8 nucleotide wing segments, respectively. The wings are composed of 2′-O-methoxyethyl nucleotides. For mouse or rat cells the positive control oligonucleotide is ISIS15770 (ATGCATTCTGCCCCCAAGGA, SEQ ID NO: 2; targeted to rodent c-raf), a chimeric oligonucleotide having a 10 nucleotide gap segment composed of 2′-deoxynucleotides, which is flanked on the 5′ side and 3′ sides by 5 nucleotide wing segments. The wings are composed of 2′-O-methoxyethyl nucleotides. Both compounds have phosphorothioate internucleoside (backbone) linkages and cytidines in the wing segments are 5-methylcytidines. The concentration of positive control oligonucleotide that results in 80% inhibition of H-ras (for ISIS13920) or c-raf (for ISIS15770) mRNA is then utilized as the screening concentration for new oligonucleotides in subsequent experiments for that cell line. If 80% inhibition is not achieved, the lowest concentration of positive control oligonucleotide that results in 60% inhibition of H-ras or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 5 nM to 300 nM.

### Example 10

### Analysis of Oligonucleotide Inhibition of Apolipoprotein B Expression

Antisense modulation of apolipoprotein B expression can be assayed in a variety of ways known in the art. For example, apolipoprotein B mRNA levels can be quantitated by, e.g., Northern blot analysis, competitive polymerase chain reaction (PCR), or real-time PCR. Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM® 7700 Sequence Detection System, available from PE-Applied Biosystems, Foster City, Calif. and used according to manufacturer's instructions.

Protein levels of apolipoprotein B can be quantitated in a variety of ways well known in the art, such as immunoprecipitation, Western blot analysis (immunoblotting), ELISA or fluorescence-activated cell sorting (FACS). Antibodies directed to apolipoprotein B can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 11

### Poly(A)+ mRNA isolation

Poly(A)+ mRNA was isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C. was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

### Example 12

### Total RNA Isolation

Total RNA was isolated using an RNEASY® 96 kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 100 μL Buffer RLT was added to each well and the plate vigorously agitated for 20 seconds. 100 μL of 70% ethanol was then added to each well and the contents mixed by pipetting three times up and down. The samples were then transferred to the RNEASY® 96 well plate attached to a QIAvac manifold fitted with a waste collection tray and attached to a vacuum source. Vacuum was applied for 15 seconds. 1 mL of Buffer RW1 was added to each well of the RNEASY® 96 μlate and the vacuum again applied for 15 seconds. 1 mL of Buffer RPE was then added to each well of the RNEASY® 96 μlate and the vacuum applied for a period of 15 seconds. The Buffer RPE wash was then repeated and the vacuum was applied for an additional 10 minutes. The plate was then removed from the QIAvac manifold and blotted dry on paper towels. The plate was then re-attached to the QIAvac manifold fitted with a collection tube rack containing 1.2 mL collection tubes. RNA was then eluted by pipetting 60 μL water into each well, incubating 1 minute, and then applying the vacuum for 30 seconds. The elution step was repeated with an additional 60 μl, water.

The repetitive pipetting and elution steps may be automated using a QIAGEN® Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

### Real-Time Quantitative PCR Analysis of Apolipoprotein B mRNA Levels

Quantitation of apolipoprotein B mRNA levels was determined by real-time quantitative PCR using the ABI PRISM® 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE™ FAM™, or VIC™, obtained from either Operon Technologies Inc., Alameda, Calif. or PE-Applied Biosystems, Foster City, Calif.) is attached to the 5′ end of the probe and a quencher dye (e.g., TAMRAT™, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM® 7700 Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μl, PCR cocktail (2.5×PCR buffer minus MgCl₂, 6.6 mM MgCl₂, 375 μl\A each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time PCR are normalized using either the expression level of GAPDH, a gene whose expression is constant, or by quantifying total RNA using RIBOGREEN® (Molecular Probes, Inc. Eugene, Oreg.). GAPDH expression is quantified by real time PCR, by being run simultaneously with the target, multiplexing, or separately. Total RNA is quantified using RIBOGREEN® RNA quantification reagent from Molecular Probes. Methods of RNA quantification by RIBOGREEN® are taught in Jones, L. J., et al, Analytical Biochemistry, 1998, 265, 368-374.

In this assay, 175 μL of RIBOGREEN® working reagent (RIBOGREEN® reagent diluted 1:2865 in 10 mM Tris-HCl, 1 mM EDTA, pH 7.5) is pipetted into a 96-well plate containing 25 uL purified, cellular RNA. The plate is read in a CytoFluor 4000 (PE Applied Biosystems) with excitation at 480 nm and emission at 520 nm.

Probes and primers to human apolipoprotein B were designed to hybridize to a human apolipoprotein B sequence, using published sequence information (GENBANK® accession number NM 000384.1, incorporated herein as SEQ ID NO: 3). For human apolipoprotein B the PCR primers are:

forward primer: TGCTAAAGGCACATATGGCCT (SEQ ID NO: 4)

reverse primer: CTCAGGTTGGACTCTCCATTGAG (SEQ ID NO: 5) and the PCR probe is: FAM-CTTGTCAGAGGGATCCTAACACTGGCCG-TAMRA (SEQ ID NO: 6) where FAM™ (PE-Applied Biosystems, Foster City, Calif.) is the fluorescent reporter dye) and TAMRA™ (PE-Applied Biosystems, Foster City, Calif.) is the quencher dye.

For human GAPDH the PCR primers are:

forward primer: GAAGGTGAAGGTCGGAGTC (SEQ ID NO: 7)

reverse primer: GAAGATGGTGATGGGATTTC (SEQ ID NO: 8) and the PCR probe is: 5′ JOE-CAAGCTTCCCGTTCTCAGCC-TAMRA 3′ (SEQ ID NO: 9) where JOE™ (PE-Applied Biosystems, Foster City, Calif.) is the fluorescent reporter dye) and TAMRA™ (PE-Applied Biosystems, Foster City, Calif.) is the quencher dye.

Probes and primers to mouse apolipoprotein B were designed to hybridize to a mouse apolipoprotein B sequence, using published sequence information (GENBANK® accession number M35186, incorporated herein as SEQ ID NO: 10). For mouse apolipoprotein B the PCR primers are:

forward primer: CGTGGGCTCCAGCATTCTA (SEQ ID NO: 11)

reverse primer: AGTCATTTCTGCCTTTGCGTC (SEQ ID NO: 12) and the PCR probe is: FAM-CCAATGGTCGGGCACTGCTCAA-TAMRA SEQ ID NO: 13) where FAM™ (PE-Applied Biosystems, Foster City, Calif.) is the fluorescent reporter dye) and TAMRA™ (PE-Applied Biosystems, Foster City, Calif.) is the quencher dye. For mouse GAPDH the PCR primers are:

forward primer: GGCAAATTCAACGGCACAGT (SEQ ID NO: 14)

reverse primer: GGGTCTCGCTCCTGGAAGAT (SEQ ID NO:15) and the PCR probe is: 5′ JOE-AAGGCCGAGAATGGGAAGCTTGTCATC-TAMRA 3′ (SEQ ID NO: 16) where JOE™ (PE-Applied Biosystems, Foster City, Calif.) is the fluorescent reporter dye) and TAMRA™ (PE-Applied Biosystems, Foster City, Calif.) is the quencher dye.

### Example 14

### Northern Blot Analysis of Apolipoprotein B mRNA Levels

Eighteen hours after antisense treatment, cell monolayers were washed twice with cold PBS and lysed in 1 mL RNAZOL® (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND®-N+nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER® UV Crosslinker 2400 (Stratagene, Inc, La Jolla, Calif.) and then robed using QUICKHYB® hybridization solution (Stratagene, La Jolla, Calif.) using manufacturer's recommendations for stringent conditions.

To detect human apolipoprotein B, a human apolipoprotein B specific probe was prepared by PCR using the forward primer TGCTAAAGGCACATATGGCCT (SEQ ID NO: 4) and the reverse primer CTCAGGTTGGACTCTCCATTGAG (SEQ ID NO: 5). To normalize for variations in loading and transfer efficiency membranes were stripped and probed for human glyceraldehyde-3-phosphate dehydrogenase (GAPDH) RNA (Clontech, Palo Alto, Calif.).

To detect mouse apolipoprotein B, a human apolipoprotein B specific probe was prepared by PCR using the forward primer CGTGGGCTCCAGCATTCTA (SEQ ID NO: 11) and the reverse primer AGTCATTTCTGCCTTTGCGTC (SEQ ID NO: 12). To normalize for variations in loading and transfer efficiency membranes were stripped and probed for mouse glyceraldehyde-3-phosphate dehydrogenase (GAPDH) RNA (Clontech, Palo Alto, Calif.).

Hybridized membranes were visualized and quantitated using a PHOSPHORIMAGER® and IMAGEQUANT® Software V3.3 (Molecular Dynamics, Sunnyvale, Calif.). Data was normalized to GAPDH levels in untreated controls.

### Example 15

Microarray analysis: evaluation of dose-dependent gene expression patterns in lean versus high-fat fed mice

DNA array analysis of gene expression patterns is a useful tool for investigating global mRNA changes following antisense inhibition of a target gene. To this end, gene expression patterns in mouse liver were evaluated following antisense inhibition of apolipoprotein B. ISIS147764 and ISIS147483 are targeted to mouse apolipoprotein B and were the antisense compounds used in this study. ISIS147764 (GTCCCTGAAGATGTCAATGC, SEQ ID NO: 17) and ISIS147483 (ATGTCAATGCCACATGTCCA, SEQ ID NO: 18) were designed using published mouse apolipoprotein B sequence (SEQ ID NO: 10). ISIS141923 (CCTTCCCTGAAGGTTCCTCC, SEQ ID NO: 19) does not target apolipoprotein B and was used as a control antisense oligonucleotide. These compounds are chimeric oligomeric compounds 20 nucleotides in length, composed of a central gap region consisting of 10 2′-deoxynucleotides, which is flanked on both sides (5′ and 3′ directions) by 5-nucleotide “wing” segments. The wings are composed of 2′-O-methoxylethyl nucleotides, or 2′-MOE nucleotides. The internucleoside (backbone) linkages are phosphorothioate throughout, and all cytidine residues are 5-methylcytidines.

Liver gene expression patterns were evaluated as a function of apolipoprotein B antisense oligonucleotide dose. Male C57B1/6 mice were divided into the following groups: (1) mice on a lean diet, injected with saline (lean control); (2) mice on a high fat diet, injected with saline (high-fat fed); (3) mice on a high fat diet injected with 50 mg/kg of the control oligonucleotide 141923 (SEQ ID NO: 19); (4) mice on a high fat diet given 20 mg/kg atorvastatin calcium (Lipitor®, Pfizer Inc.); (5) mice on a high fat diet injected with 10, 25 or 50 mg/kg ISIS147764 (SEQ ID NO: 17) (6) mice on a high fat diet injected with 10, 25 or 50 mg/kg ISIS147483 (SEQ ID NO: 18). Each dose of apolipoprotein B antisense oligonucleotide was administered to a total of 5 mice, thus groups (5) and (6) consisted of 15 animals each. All other groups consisted of 5 animals each. Mice in the high-fat diet groups were maintained on a diet of 60% lard for 4 weeks prior to treatment. Saline and oligonucleotide treatments were administered intraperitoneally twice weekly for 6 weeks. Atorvastatin was administered daily for 6 weeks. At study termination, liver samples were isolated from each animal and RNA was isolated for Northern blot qualitative assessment, DNA microarray and quantitative real-time PCR. Northern blot assessment and quantitative real-time PCR were performed as described herein.

Mouse apolipoprotein B mRNA expression, measured by real-time PCR, was evaluated to confirm antisense inhibition by ISIS147764 and ISIS147483. Serum cholesterol levels, measured by routine clinical analysis (for example, using an Olympus AU640e Chemistry Immuno Analyzer, Olympus, Melville, N.Y.) were also determined. Both apolipoprotein B mRNA and serum cholesterol levels were lowered in a dose-dependent manner following treatment with ISIS147764 or ISIS147483. The 50 mg/kg dose of ISIS147483 increased ALT and AST levels. The 10, 25 and 50 mg/kg doses of ISIS 147764 and the 10 and 25 mg/kg doses of ISIS147483 did not significantly elevate ALT or AST levels, indicating that the treatment did not result in toxicity.

DNA microarray analysis was performed using Affymetrix® gene expression analysis arrays, instruments and software tools, according to the manufacturer's instructions. Hybridization samples were prepared from 10 μg of total RNA isolated from each mouse liver according to the Affymetrix® Expression Analysis Technical Manual (Affymetrix, Inc., Santa Clara, Calif.). Samples were hybridized to a mouse gene chip containing approximately 22,000 genes (GENECHIP® Mouse Genome 430A 2.0 Array), which was subsequently washed and double-stained using the Fluidics Station 400 (Affymetrix, Inc., Santa Clara, Calif.) as defined by the manufacturer's protocol. Stained gene chips were scanned for probe cell intensity with the GENECHIP® Scanner (Affymetrix, Inc., Santa Clara, Calif.). Signal values for each probe set were calculated using the Affymetrix® Microarray Suite v5.0 software (Affymetrix, Inc., Santa Clara, Calif.). Each condition was profiled from 5 biological samples per group, one chip per sample. Fold change in expression was computed using the geometric mean of signal values as generated by Affymetrix® Microarray Suite v5.0. Statistical analysis utilized one-way ANOVA followed by 9 pair-wise comparisons. All groups were compared to the high fat group to determine gene expression changes resulting from ISIS147764 and ISIS147483 treatment. Fold changes in gene expression for genes on the chip are described in the tables provided in U.S. Provisional Application Ser. No. 60/568,825, which are herein incorporated by reference in their entirety: modified_GeneList_APOBOnly.xls, modified_GeneListAtor Only.xls, modified_AtorAPOB.xls, and modified_GeneListNonSpecific.xls.

Microarray data was interpreted using hierarchical clustering and principal component analysis to visualize global gene expression patterns. Principal component analysis (PCA) involves a mathematical procedure that transforms a number of (possibly) correlated variables into a (smaller) number of uncorrelated variables called principal components. The first principal component accounts for as much of the variability in the data as possible, and each succeeding component accounts for as much of the remaining variability as possible. Hierarchical clustering is a multivariate technique useful in identifying distinct groups in the data, in such a way that objects belonging to the same cluster resemble each other, whereas objects in different clusters are dissimilar. Statistical analyses of the microarray data in the dose-dependence study are further described in U.S. Provisional Application Ser. No. 60/568,825, (“MicroArrayReport 7.pdf”), which is herein incorporated by reference in its entirety.

Both hierarchical clustering and PCA revealed that treatment with ISIS147764 shifts the gene expression profile in high fat fed mice to the profile observed in lean mice. Thus, antisense inhibition of apolipoprotein B shifts a gene expression profile of an obese animal to that of a lean animal in a dose-dependent fashion.

### Example 16

### Microarray Analysis: Evaluation of Time-Dependent Gene Expression Patterns in Lean Versus High-Fat Fed Mice

In a further embodiment, the effects of antisense inhibition of apolipoprotein B as a function of time were investigated using DNA microarray analysis. In this study, microarray analyses of liver gene expression patterns were performed following 48 hours, 1 week, 2 weeks and 4 weeks of treatment. Male C57B1/6 mice were divided into the following groups: (1) mice on a lean diet, injected with saline (lean control); (2) mice on a high fat diet (high-fat fed); (3) mice on a high fat diet injected with 50 mg/kg of the control oligonucleotide 141923 (SEQ ID NO: 19); (4) mice on a high fat diet given 20 mg/kg atorvastatin calcium (Lipitor®, Pfizer Inc.); (5) mice on a high fat diet injected with 10, 25 or 50 mg/kg ISIS147764 (SEQ ID NO: 17). Mice in the high-fat diet groups were maintained on a diet of 60% lard for 4 weeks prior to treatment. Saline and oligonucleotide treatments were administered intraperitoneally twice weekly throughout the treatment period. Atorvastatin was administered daily throughout the treatment period. Each individual dose, time and treatment group consisted of 8 animals. Animals were sacrificed and liver samples were procured after 48 hours, 1 week, 2 weeks or 4 weeks of treatment. RNA was isolated from liver tissue for Northern blot qualitative assessment, DNA microarray and quantitative real-time PCR. Northern blot assessment and quantitative real-time PCR were performed as described herein. DNA microarray analysis was performed as described for the 6 week dose-dependence study. All groups were compared to the high fat group to determine gene expression changes resulting from ISIS147764 and ISIS147883 treatment.

For the time-dependence study, fold changes in gene expression for genes on the chips are described in the table provided in U.S. Provisional Application Ser. No. 60/568,825, which is herein incorporated by reference in its entirety: modified_MGraham_TimeCourse.xls.

Statistical analyses were carried out as described for the dose-dependence study, and are further described in U.S. Provisional Application Ser. No. 60/568,825, (MicroArray Report 11.doc) which is herein incorporated by reference in its entirety.

Analysis of the microarray data from the time dependence study revealed that, as was observed in the dose-dependence study, the gene expression profile following treatment with ISIS147764 shifts from that of high-fat fed mice to that of a lean mouse. Thus, antisense inhibition of apolipoprotein B shifts a gene expression profile of an obese animal to that of a lean animal in a time-dependent manner.

### Example 17

### Gene Expression Changes Induced by Antisense Inhibition of Apolipoprotein B

Differentially expressed genes were classified according to gene family assignments in the Gene Ontology database. Comparison of the ISIS147764-treated samples from the dose-dependence study with the ISIS147764-treated samples from the time-dependence study revealed that many genes involved in metabolic processes were concurrently down-regulated as a function of both antisense oligonucleotide dose and length of treatment. Gene families with members down-regulated in a dose- and time-dependent manner are those of lipid metabolism, lipid biosynthesis, fatty acid biosynthesis, fatty acid binding proteins, phosphotidylcholine biosynthesis, steroid biosynthesis, lipid transport, glycogen synthesis, gluconeogenesis, complement activation, acute phase response, inflammatory response, pro-apoptosis and anti-apoptosis. Gene families with members upregulated in a dose and time-dependent manner following apolipoprotein B antisense inhibition included lipid metabolism, fatty acid biosynthesis, steroid biosynthesis, cholesterol metabolism, complement activation, acute phase response, inflammatory response, matrix metalloproteinases and pro-apoptosis. Some gene families, for example, lipid metabolism, contained both up- and down-regulated genes.

Gene expression changes for a subset of genes analyzed by DNA microarray in both the dose- and time-dependence studies are presented by gene family in Tables 1, 2, 3, 4, and 5. Gene names used are the official symbols from the National Center for Biotechnology Information (NCBI). GENBANK® accession numbers corresponding to gene symbols are provided in the tables in U.S. Provisional Application Ser. No. 60/568,825, which is herein incorporated by reference in its entirety. “Lean” indicates data from mice on a lean diet receiving saline treatment. “141923” indicates data from animals treated with the control oligonucleotidie ISIS141923. “ISIS147764” indicates data from the high-fat fed mice treated with ISIS147764 in the dose dependence study. “ISIS147764 50 mg/kg” indicates data from the high-fat fed mice treated with ISIS147765 in the time-dependence study. The data shown in this table represent the fold change of the indicated sample relative to samples from high-fat fed mice receiving saline treatment. For example, in high-fat fed mice receiving a 50 mg/kg dose of ISIS147764 in the dose-dependence study, Lcat gene expression experienced a fold change of −1.29 relative to gene expression levels in high-fat fed mice receiving saline treatment in the same study, i.e. ISIS147764 treatment reduce liver Lcat gene expression by 1.29 fold.

Fold changes less than or equal to −1.1 or greater than or equal to 1.1 (decrease or increase in gene expression level, respectively) that have a P-value of less than or equal to 0.05 are underscored. Fold changes with a P-value less than or equal to 0.05 are considered have the highest statistical significance. For example, the −1.29 fold reduction in Lcat gene expression is highly statistically significant. Fold changes less than or equal to −1.1 or greater than or equal to 1.1 that have a P-value of greater than 0.05 are presented in plain type. P-values for fold changes between −1.1 and 1.1 are not indicated.

The Mouse Genome 430A 2.0 Array used for these studies contains multiple probe sets for some genes. For these genes, results from each individual probe set are shown in Tables 1, 2, 3, 4 and 5. For example, in Table 1, Lip1 expression was measured by 2 probe sets, and the results from each probe set are shown in separate rows in the table.

TABLE 1
Lipid Biosynthesis and Metabolism Gene Changes

| | | 141923 | ISIS 147764 | | | ISIS 147764 (50 mg/kg) | | | |
|---|---|---|---|---|---|---|---|---|---|
| | | 50 | 10 | 25 | 50 | 48 | 1 | 2 | 4 |
| Gene | Lean | mg/kg | mg/kg | mg/kg | mg/kg | hr | week | week | week |
| Lcat | 1.04 | −1.12 | −1.21 | −1.33 | −1.29 | 1.11 | 1.12 | −1.21 | −1.15 |
| Lip1 | −1.01 | −1.06 | −1.25 | −1.41 | −1.19 | −1.36 | −1.03 | −1.25 | −2.04 |
| Lip1 | 1.55 | −1.15 | −1.63 | −1.49 | −1.25 | −1.09 | −1.28 | −1.3 | −1.6 |
| Lipc | 1.33 | −1.12 | −2.15 | −4.17 | −6.59 | −1.15 | −1.83 | −2.36 | −5.96 |
| Ppara | −2.04 | −1.08 | −1.09 | −1.09 | −1.42 | 1.01 | 1.02 | 1.12 | −1.37 |
| Pparg | −2.35 | −2.35 | −2.15 | −6.99 | −5.46 | −1.85 | 1.36 | 1.44 | −4.95 |
| Pcx | −1.14 | −1.23 | −1.24 | −1.44 | −1.5 | −1.04 | −1.22 | −1.17 | −1.77 |

TABLE 2
Cholesterol/Lipid Transport Gene Changes

| | | 141923 | ISIS 147764 | | | ISIS 147764 (50 mg/kg) | | | |
|---|---|---|---|---|---|---|---|---|---|
| | | 50 | 10 | 25 | 50 | 48 | 1 | 2 | 4 |
| Gene | Lean | mg/kg | mg/kg | mg/kg | mg/kg | hr | week | week | week |
| Apoa4 | −3.39 | −1.27 | −1.37 | −3.86 | −3.83 | −1.19 | −1.17 | −1.59 | −3.35 |
| Apoa4 | −2.48 | 1.03 | −1.04 | −2.99 | −2.68 | −1.26 | −1.17 | −1.38 | −3.13 |
| Apoc1 | −1.11 | −1.08 | −1.07 | −1.14 | −1.19 | 1.04 | 1.03 | −1.01 | −1.04 |
| Apoc2 | −1.49 | −1.17 | −1.18 | −1.39 | −1.35 | −1.19 | −1.16 | −1.15 | −1.28 |
| Apoc4 | −1.19 | 1.01 | 1.01 | −1.14 | −1.4 | 1 | −1.02 | −1 | −1.12 |
| Mttp | −1.34 | −1.33 | −1.12 | −1.12 | −1.03 | −1.13 | −1.01 | −1.11 | −1.05 |
| Mttp | −1.08 | −1.04 | −1.01 | −1.1 | −1.18 | −1.12 | 1.01 | 1.01 | −1.05 |

TABLE 3
Fatty Acid Biosynthesis/Binding Proteins Gene Changes

| | | 141923 | ISIS 147764 | | | ISIS 147764 (50 mg/kg) | | | |
|---|---|---|---|---|---|---|---|---|---|
| | | 50 | 10 | 25 | 50 | 48 | 1 | 2 | 4 |
| Gene | Lean | mg/kg | mg/kg | mg/kg | mg/kg | hr | week | week | week |
| Prkab1 | 1.29 | −1.13 | 1.04 | 1.25 | 1.29 | 1.13 | −1.04 | 1.03 | 1.08 |
| Prkag1 | −1.12 | 1.03 | −1.14 | 1.09 | 1.06 | −1.18 | −1.26 | 1.01 | 1.29 |
| Srebp-1 | −1.35 | −1.35 | −1.47 | −1.7 | −1.8 | −1.09 | −1.37 | −1.49 | −2.95 |
| Scd2 | 1.24 | 1.43 | 1.5 | 1.66 | 1.93 | 1.11 | 1.02 | −1.12 | 1.38 |
| Scd2 | 1.07 | −1.22 | 1.06 | 1.37 | 1.15 | −1.09 | 1.03 | 1.04 | 1.16 |
| Scd1 | 1.12 | −1.48 | −2.04 | −6.49 | −3.81 | −1.22 | −1.36 | −1.59 | −11.66 |
| Scd1 | −1.03 | −4.19 | −4.87 | −13.89 | −11.65 | −2.11 | −2.53 | −3.25 | −35.33 |
| Acadl | −1.05 | −1.1 | 1 | −1.11 | −1.28 | −1.01 | −1.02 | 1.14 | −1.23 |
| Acadm | −1.11 | −1.14 | 1.02 | −1.2 | −1.3 | 1.01 | 1.04 | 1.06 | −1.24 |
| Acads | −1.16 | 1.08 | −1.01 | −1.09 | −1.29 | −1.09 | 1.06 | 1.13 | −1.06 |
| Acox1 | −1.12 | −1.45 | −1.12 | −1.23 | −1.43 | 1.01 | −1.01 | 1.01 | −1.19 |
| Acox1 | −1.39 | −2.03 | −1.3 | −1.36 | −1.64 | 1 | −1.06 | 1.02 | −1.49 |
| Cpt1a | 1.37 | 1.43 | 1.31 | 1.06 | −1.74 | 1.07 | −1.33 | 1.06 | −1.11 |
| Cpt1a | −1.31 | −1.25 | −1.24 | −1.34 | −1.74 | −1.07 | −1.13 | −1.14 | −1.68 |
| Cpt1a | 1.03 | 1.22 | 1.11 | −1.08 | −1.59 | 1 | −1.24 | 1.02 | −1.9 |
| Cpt2 | −1.18 | −1.1 | −1.16 | −1.08 | −1.2 | 1.1 | 1.1 | 1.08 | −1.04 |
| Crat | −1.07 | −1.36 | −1.35 | −1.77 | −2.68 | −1.22 | −1.08 | −1.21 | −2.39 |
| Elovl2 | −1.2 | 1.01 | −1.34 | −1.38 | −2.5 | −1.12 | −1.34 | −1.38 | −2.53 |
| Elovl3 | −9.91 | 1.74 | −1.18 | −1.43 | −2.27 | −1.35 | −1.08 | −1.27 | −1.91 |
| Acadsb | −1.18 | 1.08 | −1.25 | −1.88 | −2.43 | −1.12 | −1.19 | −1.44 | −1.79 |
| Fads2 | −1.83 | −2.16 | −2.93 | −4.44 | −5.64 | −1.38 | −1.68 | −2.51 | −6.83 |
| Fasn | 1.17 | −1.2 | −1.05 | −1.96 | −1.35 | −1.18 | −1.11 | −1.37 | −3.14 |
| Facl2 | −1.3 | −1.41 | −1.31 | −1.4 | −1.65 | −1.16 | −1.26 | −1.36 | −1.44 |
| Facl2 | −1.3 | −1.23 | −1.19 | −1.27 | −1.69 | −1.08 | −1.25 | −1.16 | −1.2 |
| Facl4 | −1.5 | −1.07 | 1.23 | 1.59 | 1.71 | −1.05 | 1.19 | 1.26 | 1.9 |
| Abcd2 | 1.56 | −10.58 | −11.39 | −28.14 | −39.3 | −2.4 | −6.01 | −3.83 | −35.7 |
| Dbi | −1.15 | −1.11 | −1.15 | −1.26 | −1.5 | −1.09 | −1.12 | 1.09 | −1.2 |
| Dbi | −1.05 | −1.2 | −1.19 | −1.56 | −1.56 | −1.08 | −1.02 | −1.06 | −1.27 |
| Dbi | 1.04 | −1.16 | −1.14 | −1.29 | −1.48 | −1.21 | 1.02 | 1.08 | −1.15 |
| Dbi | −1.09 | −1.05 | −1.15 | −1.36 | −1.32 | −1.15 | −1.09 | 1.01 | −1.18 |
| Fabp1 | −1.16 | −1.12 | −1.11 | −1.11 | −1.46 | 1.26 | 1.11 | −1.02 | −1.04 |
| Fabp1 | −1.27 | −1.18 | −1.09 | −1.14 | −1.29 | 1.07 | 1.12 | 1.07 | 1.01 |
| Fabp2 | −3.46 | −1.2 | −1.82 | −3.88 | −4.87 | −1.48 | −1.76 | −1.4 | −4.74 |
| Fabp7 | −1.68 | 1.26 | 1.07 | −1.18 | 1.54 | 1.3 | 1.58 | 1.25 | 1.76 |

TABLE 4
Cholesterol Metabolism Gene Changes

| | | 141923 | ISIS 147764 | | | ISIS 147764 (50 mg/kg) | | | |
|---|---|---|---|---|---|---|---|---|---|
| | | 50 | 10 | 25 | 50 | 48 | 1 | 2 | 4 |
| Gene | Lean | mg/kg | mg/kg | mg/kg | mg/kg | hr | week | week | week |
| Acat-1 | −1.63 | −1.63 | −145 | −1.94 | −3.17 | −1.18 | −1.43 | −1.13 | −2.49 |
| Acat-1 | −1.39 | −1.29 | −1.22 | −1.49 | −4.49 | −1.12 | −1.15 | −1.08 | −1.75 |
| Acat-1 | −1.31 | −1.31 | −1.3 | −1.64 | −2.73 | −1.27 | −1.15 | −1.06 | −1.94 |
| Acca-1 | −1.12 | −1.2 | −1.11 | −1.16 | −1.31 | −1.11 | 1.06 | −1.09 | −1.46 |
| Cyp7a1 | 1.02 | −1.53 | −1.39 | −1.09 | −1.87 | 1.28 | 1.2 | −1.92 | −1.73 |
| Cyp7b1 | −4.68 | 2.52 | 1.57 | 2.02 | 1.4 | 1.06 | 1.42 | −1.01 | 2 |
| Cyp7b1 | −5.47 | 1.88 | 1.34 | 1.77 | 1.24 | −1.1 | 1.4 | −1.07 | 1.81 |
| Soat2 | 1.01 | −1.52 | 1.02 | 1.33 | 1.32 | 1.12 | 1.18 | 1.45 | 1.15 |
| Ldlr | 1.07 | 1.07 | −1.34 | −1.71 | −1.4 | −1.12 | −1.11 | −1.38 | −1.9 |
| Hmgcs1 | −1.01 | −1.01 | −1.29 | −2.06 | −1.66 | −1.01 | 1.31 | −1.06 | −2.21 |
| Hmgcs1 | 1.02 | 1.02 | −1.44 | −1.72 | −1.7 | −1.1 | 1.28 | −1.2 | −2.07 |
| Hmgcs1 | 1.05 | 1.05 | −1.39 | −1.78 | −1.56 | −1.13 | 1.24 | −1.16 | −1.84 |
| Hmgcs1 | −1.05 | −1.05 | −1.47 | −1.85 | −1.74 | −1.11 | 1.16 | −1.23 | −2.26 |
| Hmgcs2 | −1.31 | −1.31 | −1.07 | −1.23 | −1.61 | 1.03 | 1.17 | 1.13 | −1.39 |

TABLE 5
Glucose/Glycogen Synthesis Gene Changes

| | | 141923 | ISIS 147764 | | | ISIS 147764 (50 mg/kg) | | | |
|---|---|---|---|---|---|---|---|---|---|
| | | 50 | 10 | 25 | 50 | 48 | 1 | 2 | 4 |
| Gene | Lean | mg/kg | mg/kg | mg/kg | mg/kg | hr | week | week | week |
| Car5a | 1.03 | 1.01 | −1.15 | −1.18 | −1.47 | 1.04 | 1.01 | −1.06 | −1.4 |
| Gck | −2.74 | −2.74 | −2.01 | −3.39 | −11.23 | −1.28 | −1.4 | −1.78 | −7.34 |
| Gck | −1.65 | −1.65 | −1.45 | −1.93 | −3.64 | −1.12 | −1.03 | −1.53 | −3.16 |
| G6pc | −1.17 | −1 | −1.11 | −3.69 | −3.09 | −1.11 | 1.53 | −1.33 | −3.09 |

Real-time PCR analysis confirmed the reduction in mRNA expression for the following genes involved in lipid metabolism: ATP-binding cassette, sub-family D (ALD) member 2 (ABCD2), intestinal fatty acid binding protein 2 (FABP2), stearol CoA desaturase-1 (SCD1) and HMG CoA reductase (HMGCR). Probes and primers were designed to hybridize to these genes, using publicly available sequences. Probes and primers for real-time PCR can be designed using commercially available tools, for example, Primer Express® software (Applied Biosystems, Foster City, Calif.). Real-time PCR was performed as described herein, and results were normalized to GAPDH real-time PCR results. Results are presented in Table 6 and are normalized to mRNA levels from high-fat fed mice.

TABLE 6
Real-time PCR confirmation of gene expression changes following
antisene inhibition of apolipoprotein B in mice

| | | % Expression, normalized to | | | |
|---|---|---|---|---|---|
| | | high−fat diet, saline treated mice | | | |
| Diet | Treatment | ABCD2 | SCD1 | HMGCR | FABP2 |
| Lean | Saline | 193 | 64 | 117 | 28 |
| High Fat | 141923 | 32 | 43 | 131 | 109 |
| High Fat | 147764, 10 mg/kg | 52 | 25 | 109 | 66 |
| High Fat | 147764, 25 mg/kg | 5 | 4 | 102 | 32 |
| High Fat | 147764, 50 mg/kg | 7 | 3 | 207 | 22 |
| High Fat | 147483, 10 mg/kg | 42 | 27 | 91 | 71 |
| High Fat | 147483, 25 mg/kg | 70 | 19 | 135 | 74 |
| High Fat | 147483, 50 mg/kg | 71 | 29 | 163 | 96 |
| High Fat | Atorvastatin | 69 | 25 | 358 | 63 |

These results confirm the reduction in ABCD2, SCD1 and FABP2 gene expression as a result of inhibition of apolipoprotein B following treatment with ISIS147764.

Real-time PCR analysis confirmed the reduction in mRNA expression for the following additional genes involved in lipid metabolism: hepatic lipase, fatty acid synthase, HMG-CoA synthase 2 (HMGCS2), diazepam binding inhibitor (DBI), fatty acid Coenzyme A ligase, long chain 2 (FACL2), fatty acid-Coenzyme A ligase, long chain 4 (FACL4), fatty acid synthase (FASN), glucose-6-phosphatase, catalytic subunit (G6PC), hydroxysteroid (17-beta) dehydrogenase 12 (HSD17b12), low density lipoprotein receptor (LDLr), microsomal triglyceride transfer protein (MTP or MTTP), pyruvate carboxylase (PCX), peroxisome proliferator activated receptor-gamma (PPAR-gamma), matrix metalloproteinase-12 (MMP-12), activating transcription factor 5 (ATFS) and Bc12-associated X protein (BAX).

Together, these gene expression studies reveal that antisense inhibition of apolipoprotein B can modulate a number of downstream events in several different gene pathways. Treatment of high-fat fed mice with an antisense inhibitor of apolipoprotein B shifted the gene expression profile to resemble that of a mouse on a lean diet. Thus, antisense inhibitors of apolipoprotein B are candidate therapeutic agents for the treatment of conditions characterized by abnormal lipid metabolism, such as hyperlipidemia, or conditions that increase cardiovascular disease risk, such as obesity.

### Example 18

### AMPK Activation Following Antisense Inhibition of Apolipoprotein B

Additional analyses of gene expression profiles from mice treated with antisense oligonucleotide targeted to apolipoprotein B revealed an increase in AMP-activated protein kinase (AMPK). AMPK is the downstream component of a kinase cascade that acts as a sensor for glucose and lipid metabolism. AMPK is a ubiquitous serine/threonine kinase activated in response to environmental or nutritional stress factors which deplete intracellular ATP levels, including heat shock, hypoxia, hypoglycemia and prolonged exercise. The result of AMPK activation is the inhibition of energy-consuming biosynthetic pathways, such as fatty acid and sterol synthesis, and activation of ATP-producing catabolic pathways, such as fatty acid oxidation. AMPK exists as a heterotrimer, comprising a catalytic alpha subunit and regulatory beta and gamma subunits. In mammals, each subunit is encoded by multiple genes: alpha 1, alpha 2, beta 1, beta 2, gamma 1, gamma 2 and gamma 3 (reviewed in Kahn, et al., Cell Metabolism, 2005, 1, 15-25).

The microarray analyses described herein revealed that AMPK beta 1 (gene symbol Prkab1) and gamma 1 (gene symbol Prkag1) regulatory subunits were increased following treatment with ISIS147764. Real-time PCR analysis of liver samples from both the dose-dependence and time-dependence studies revealed that AMPK alpha 2 (gene symbol Prkaa2) expression was elevated as well. Relative to expression in high-fat fed mice treated with saline, AMPK alpha 2 expression was increased by 41%, 49%, and 87% in animals treated twice weekly with 10, 25 and 50 mg/kg ISIS147754, respectively, whereas AMPK alpha 2 expression was elevated by 25% and 8% in lean, saline-treated and ISIS141923-treated animals, respectively. AMPK alpha 2 was similarly increased at the end of the time-dependence study, at which time AMPK alpha 2 levels were 31% greater in mice treated with 50 mg/kg ISIS147764 twice weekly, relative to high fat fed mice treated with saline. In an additional study, in which mice were treated with ISIS147764 at a dose of 50 mg/kg per week, twice weekly, for a period of 3 months, AMPK alpha 1 liver protein levels were increased by 2.4 fold relative to saline-treated animals (as determined by routine western blotting). These data illustrate that the levels of AMPK subunits, including the catalytic alpha subunits, are increased as a result of antisense inhibition of apolipoprotein B.

The increase in AMPK subunits is gene expression profile change characteristic of a lean animal; this gene profile change provides an additional marker for assessing shifts in gene expression profile following antisense inhibition of apolipoprotein B. Activation of AMPK is known to inhibit energy-consuming biosynthetic pathways, such as fatty acid and sterol synthesis, and activate ATP-producing catabolic pathways, such as fatty acid oxidation. Metformin, a drug widely used for the treatment of type 2 diabetes that also has beneficial effects on circulating lipids linked to cardiovascular risk, activates AMPK activity in cultured hepatocytes and also increases AMPK alpha 2 activityin the skeletal muscle of subjects treated with metformin, (Zhou et al., J. Clin. Invest., 2001, 108, 1167-1173; Musi, et al., Diabetes, 2002, 51, 2074-2081). Therefore, antisense oligonucleotides targeted to apolipoprotein B are candidate therapeutic agents with application in the treatment of cardiovascular disease, such as hyperlipidemia, and metabolic disorders, such as type 2 diabetes.

### Example 19

### Antisense Inhibition of Apolipoprotein B in Functional Assays

Functional assays are used to evaluate how gene expression affects cellular pathways and metabolic processes. In a further embodiment, a variety of functional assays were performed to investigate how apolipoprotein B participates in cell proliferation and survival, angiogenesis, adipocytes differentiation and the inflammatory response. Such assays can be used, by way of example, to determine the function of apolipoprotein B in different cellular pathways and metabolic processes and to identify new therapeutic areas where inhibition of apolipoprotein B can be beneficial.

The effects of antisense inhibition of apolipoprotein B on cellular pathways and metabolic processes were evaluated using ISIS147788 (TTTCTGTTGCCACATTGCCC, SEQ ID NO: 20), which targets human apolipoprotein B and was designed using publicly available sequence (SEQ ID NO: 3). ISIS147788 is a chimeric oligomeric compounds 20 nucleotides in length, composed of a central gap region consisting of 10 2′-deoxynucleotides, which is flanked on both sides (5′ and 3′ directions) by 5-nucleotide “wing” segments. The wings are composed of 2′-O-methoxylethyl nucleotides, or 2′-MOE nucleotides. The internucleoside (backbone) linkages are phosphorothioate throughout, and all cytidine residues are 5-methylcytidines.

### Cell Proliferation and Survival

Cell cycle regulation is the basis for various cancer therapeutics. Unregulated cell proliferation is a characteristic of cancer cells, thus most current chemotherapy agents target dividing cells, for example, by blocking the synthesis of new DNA required for cell division. However, cells in healthy tissues are also affected by agents that modulate cell proliferation.

In some cases, a cell cycle inhibitor will cause apoptosis in cancer cells, but allow normal cells to undergo growth arrest and therefore remain unaffected (Blagosklonny, Bioessays, 1999, 21, 704-709; Chen et al., Cancer Res., 1997, 57, 2013-2019; Evan and Littlewood, Science, 1998, 281, 1317-1322; Lees and Weinberg, Proc. Natl. Acad. Sci. USA, 1999, 96, 4221-4223). An example of sensitization to anti-cancer agents is observed in cells that have reduced or absent expression of the tumor suppressor genes p53 (Bunz et al., Science, 1998, 282, 1497-1501; Bunz et al., J. Clin. Invest., 1999, 104, 263-269; Stewart et al., Cancer Res., 1999, 59, 3831-3837; Wahl et al., Nat. Med., 1996, 2, 72-79). However, cancer cells often escape apoptosis (Lowe and Lin, Carcinogenesis, 2000, 21, 485-495; Reed, Cancer J. Sci. Am., 1998, 4 Suppl 1, S8-14). Further disruption of cell cycle checkpoints in cancer cells can increase sensitivity to chemotherapy while allowing normal cells to take refuge in G1 and remain unaffected. Cell cycle assays are employed to identify genes, such as p53, whose inhibition will sensitize cells to anti-cancer agents.

### Cell Cycle Assay

The effects of antisense inhibition of apolipoprotein B were examined in normal human mammary epithelial cells (HMECs) as well as two breast carcinoma cell lines, MCF7 and T47D. All of the cell lines are obtained from the American Type Culture Collection (Manassas, Va.). The latter two cell lines express similar genes but MCF7 cells express the tumor suppressor p53, while T47D cells are deficient in p53. MCF-7 and HMECs cells are routinely cultured in DMEM low glucose (Invitrogen Life Technologies, Carlsbad, Calif.) supplemented with 10% fetal bovine serum (Invitrogen Life Technologies, Carlsbad, Calif.). T47D cells were cultured in DMEM High glucose media (Invitrogen Life Technologies, Carlsbad, Calif.) supplemented with 10% fetal bovine serum. Cells were routinely passaged by trypsinization and dilution when they reached approximately 90% confluence. Cells were plated in 24-well plates at approximately 50,000-60,000 cells per well for HMEC cells, approximately 140,000 cells per well for MCF-7 and approximately 170,000 cells per well for T47D cells, and allowed to attach to wells overnight.

ISIS147788 (SEQ ID NO: 20) was used to inhibit apolipoprotein B mRNA expression. An oligonucleotide with a randomized sequence, ISIS 29848 (NNNNNNNNNNNNNNNNNNNN; where N is A,T,C or G; herein incorporated as SEQ ID NO: 21) was used a negative control, a compound that does not modulate cell cycle progression. In addition, a positive control for the inhibition of cell proliferation was assayed. The positive control was ISIS183881 (ATCCAAGTGCTACTGTAGTA; herein incorporated as SEQ ID NO: 22) targets kinesin-like 1 and served as a positive control for the inhibition of cell cycle progression. ISIS 29248 and ISIS183881 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 200 nM of oligonucleotide and 6 μg/mL LIPOFECTIN®. Before adding to cells, the oligonucleotide, LIPOFECTIN® and OPTI-MEM® 1 were mixed thoroughly and incubated for 0.5 hrs. The medium was removed from the plates and the plates were tapped on sterile gauze. Each well containing T47D or MCF7 cells was washed with 150 μl of phosphate-buffered saline. Each well containing HMECs was washed with 150 μL of Hank's balanced salt solution. The wash buffer in each well was replaced with 100 μL of the oligonucleotide/OPTI-MEM® 1/LIPOFECTIN® cocktail. Control cells received LIPOFECTIN® only. The plates were incubated for approximately 4 hours at 37° C., after which the medium was removed and the plate was tapped on sterile gauze. 100 μl of full growth medium was added to each well. After approximately 72 hours, routine procedures were used to prepare cells for flow cytometry analysis and cells were stained with propidium iodide to generate a cell cycle profile using a flow cytometer. The cell cycle profile was analyzed with the MODFIT™ program (Verity Software House, Inc., Topsham Me.).

Fragmentation of nuclear DNA is a hallmark of apoptosis and produces an increase in cells with a hypodiploid DNA content, which are categorized as “subG1”. An increase in cells in G1 phase is indicative of a cell cycle arrest prior to entry into S phase; an increase in cells in S phase is indicative of cell cycle arrest during DNA synthesis; and an increase in cells in the G2/M phase is indicative of cell cycle arrest just prior to or during mitosis. Data are expressed as percentage of cells in each phase relative to the cell cycle profile of untreated control cells and are shown in Table 8. Values above or below 100% indicate an increase or decrease, respectively, in each cell cycle population. For example, following treatment of MCF7 cells with ISIS147788, 109% of the cells were in G1 phase, relative to the untreated cells, demonstrating an increase of 9% in the G1 phase population and indicative of a cell cycle arrest prior to entry into S phase.

TABLE 8
Cell cycle profile of cells treated with oligomeric
compounds targeted to apolipoprotein B

| Cell | | | Sub | G1 | S | G2/M |
|---|---|---|---|---|---|---|
| Type | Treatment | Target | G1 | Phase | Phase | Phase |
| MCF7 | ISIS 147788 | apolipoprotein B | 158 | 109 | 88 | 98 |
| | ISIS 29848 | negative control | 130 | 104 | 94 | 98 |
| | ISIS 183881 | positive control | 57 | 126 | 108 | 51 |
| T47D | ISIS 147788 | apolipoprotein B | 140 | 107 | 92 | 90 |
| | ISIS 29848 | negative control | 111 | 105 | 113 | 74 |
| | ISIS 183881 | positive control | 39 | 120 | 133 | 52 |
| HMEC | ISIS 147788 | apolipoprotein B | 584 | 95 | 108 | 107 |
| | ISIS 29848 | negative control | 376 | 92 | 120 | 105 |
| | ISIS 183881 | positive control | 289 | 110 | 106 | 72 |

Treatment of MCF7 and T47D cells and HMECs with ISIS147788 did not result in a significant arrest in cell cycle progression. SubG1 populations were increased by antisense inhibition of apolipoprotein B, indicating an increase in apopoptotic cells.

### Caspase Assay

Programmed cell death, or apoptosis, is an important aspect of various biological processes, including normal cell turnover, as well as immune system and embryonic development. Apoptosis involves the activation of caspases, a family of intracellular proteases through which a cascade of events leads to the cleavage of a select set of proteins. The caspase family can be divided into two groups: the initiator caspases, such as caspase-8 and -9, and the executioner caspases, such as caspase-3, -6 and -7, which are activated by the initiator caspases. The caspase family contains at least 14 members, with differing substrate preferences (Thornberry and Lazebnik, Science, 1998, 281, 1312-1316). A caspase assay is utilized to identify genes whose inhibition selectively causes apoptosis in breast carcinoma cell lines, without affecting normal cells, and to identify genes whose inhibition results in cell death in the p53-deficient T47D cells, and not in the MCF7 cells which express p53 (Ross et al., Nat. Genet., 2000, 24, 227-235; Scherf et al., Nat. Genet., 2000, 24, 236-244). The chemotherapeutic drugs taxol, cisplatin, etoposide, gemcitabine, camptothecin, aphidicolin and 5-fluorouracil all have been shown to induce apoptosis in a caspase-dependent manner.

In a further embodiment, antisense inhibition of apolipoprotein B was examined in normal human mammary epithelial cells (HMECs) as well as two breast carcinoma cell lines, MCF7 and T47D. All cells were cultured as described for the cell cycle assay in 96-well plates with black sides and flat, transparent bottoms (Corning Incorporated, Corning, N.Y.). DMEM media, with and without phenol red, were obtained from Invitrogen Life Technologies (Carlsbad, Calif.). MEGM media, with and without phenol red, were obtained from Cambrex Bioscience (Walkersville, Md.).

ISIS147788 (SEQ ID NO: 20) was used to inhibit apolipoprotein B mRNA expression. An oligonucleotide with a randomized sequence, ISIS 29848 (NNNNNNNNNNNNNNNNNNNN; where N is A,T,C or G; incorporated herein as SEQ ID NO: 21) was used as a negative control, a compound that does not effect caspase activity. As a positive control for caspase activation, an oligonucleotide targeted to human Jagged2 ISIS148715 (TTGTCCCAGTCCCAGGCCTC; herein incorporated as SEQ ID NO: 23) or human Notch1 ISIS 226844 (GCCCTCCATGCTGGCACAGG; herein incorporated as SEQ ID NO: 24) was also assayed. Both of these genes are known to induce caspase activity, and subsequently apoptosis, when inhibited. ISIS 29248, ISIS148715 and ISIS 226844 are all chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Cells were treated as described for the cell cycle assay with 200 nM oligonucleotide in 6 μg/mL LIPOFECTIN®. Caspase-3 activity was evaluated with a fluorometric HTS Caspase-3 assay (Catalog #HTSO2; EMD Biosciences, San Diego, Calif.) that detects cleavage after aspartate residues in the peptide sequence (DEVD). The DEVD substrate is labeled with a fluorescent molecule, which exhibits a blue to green shift in fluorescence upon cleavage by caspase-3. Active caspase-3 in the oligonucleotide treated cells is measured by this assay according to the manufacturer's instructions. Approximately 48 hours following oligonucleotide treatment, 50 uL of assay buffer containing 10 μM dithiothreitol was added to each well, followed by addition 20 uL of the caspase-3 fluorescent substrate conjugate. Fluorescence in wells was immediately detected (excitation/emission 400/505 nm) using a fluorescent plate reader (SPECTRAMAX® GEMINI XS, Molecular Devices, Sunnyvale, Calif.). The plate was covered and incubated at 37° C. for and additional three hours, after which the fluorescence was again measured (excitation/emission 400/505 nm). The value at time zero was subtracted from the measurement obtained at 3 hours. The measurement obtained from the untreated control cells was designated as 100% activity.

The experiment was replicated in each of the 3 cell types, HMECs, T47D and MCF7 and the results are shown in Table 9. From these data, values for caspase activity above or below 100% are considered to indicate that the compound has the ability to stimulate or inhibit caspase activity, respectively. The data are shown as percent increase in fluorescence relative to untreated control values.

TABLE 9
Effects of antisense inhibition of apolipoprotein
B on apoptosis in the caspase assay

| | | | % Caspase activ- |
|---|---|---|---|
| | | | ity relative to |
| Cell Type | Treatment | Target | untreated control |
| MCF7 | ISIS 147788 | apolipoprotein B | 253 |
| | ISIS 148715 | positive control | 463 |
| | ISIS 29848 | negative control | 118 |
| T47D | ISIS 147788 | apolipoprotein B | 91 |
| | ISIS 148715 | positive control | 950 |
| | ISIS 29848 | negative control | 81 |
| HMEC | ISIS 147788 | apolipoprotein B | 97 |
| | ISIS 148715 | positive control | 1418 |
| | ISIS 29848 | negative control | 69 |

These results demonstrate that ISIS147788 causes a significant increase in apoptosis in MCF7 cells.

In a further embodiment, a similar caspase assay was performed to compare caspase-3 activity in T47D cells, which lack functional p53, to that in T47D cells engineered to harbor a functional p53 gene. T47D+p53 cells are T47D cells that have been transfected with and selected for maintenance of a plasmid that expresses a wildtype copy of the p53 gene (for example, pCMV-p53; Clontech, Palo Alto, Calif.), using standard laboratory procedures. The cells were treated with oligonucleotide as described for T47D cells and caspase-3 activity was measured after approximately 24 and 48 hours of treatment, as described herein. Untreated control cells served as the control to which data were normalized. The results are presented in Table 10. From these data, values for caspase activity above or below 100% are considered to indicate that the compound has the ability to stimulate or inhibit caspase activity, respectively. The data are shown as percent increase in fluorescence relative to untreated control values.

TABLE 10
Caspase activity in the presence and absence of p53,
following antisense inhibition of apolipoprotein B

| | Time | | | % Caspase activ- |
|---|---|---|---|---|
| Cell | following | | | ity relative to |
| Type | treatment | Treatment | Target | untreated control |
| T47D | 24 hours | ISIS 147788 | apolipoprotein B | 94 |
| | | ISIS 148715 | positive control | 147 |
| | | ISIS 29848 | negative control | 106 |
| T47D + | 24 hours | ISIS 147788 | apolipoprotein B | 101 |
| p53 | | ISIS 148715 | positive control | 172 |
| | | ISIS 29848 | negative control | 120 |
| T47D | 48 hours | ISIS 147788 | apolipoprotein B | 167 |
| | | ISIS 148715 | positive control | 143 |
| | | ISIS 29848 | negative control | 74 |
| T47D + | 48 hours | ISIS 147788 | apolipoprotein B | 110 |
| p53 | | ISIS 148715 | positive control | 218 |
| | | ISIS 29848 | negative control | 111 |

From these data it is evident that inhibition of apolipoprotein B expression by ISIS147788 for 48 hours resulted in a significant induction of apoptosis T47D cells without p53, compared to untreated control cells controls, whereas apoptosis was neither induced nor inhibited in cells with functional p53.

These data demonstrate that, in the absence of a wild-type p53 gene, antisense inhibition of apolipoprotein B in T47D cells leads to a greater apoptotic cell fraction than in the presence of functional p53. Thus, the reintroduction of p53 into T47D cells resulted in decreased sensitivity of the cells to antisense inhibition of apolipoprotein B. Therefore, the inhibition of apolipoprotein B expression can be used to selectively modulate the growth of p53-deficient cells, such as cancer cells.

### Angiogenesis Assays

Angiogenesis is the growth of new blood vessels (veins and arteries) by endothelial cells. This process is important in the development of a number of human diseases, and is believed to be particularly important in regulating the growth of solid tumors. Without new vessel formation it is believed that tumors will not grow beyond a few millimeters in size. In addition to their use as anti-cancer agents, inhibitors of angiogenesis have potential for the treatment of diabetic retinopathy, cardiovascular disease, rheumatoid arthritis and psoriasis (Carmeliet and Jain, Nature, 2000, 407, 249-257; Freedman and Isner, J. Mol. Cell. Cardiol., 2001, 33, 379-393; Jackson et al., Faseb J., 1997, 11, 457-465; Saaristo et al., Oncogene, 2000, 19, 6122-6129; Weber and De Bandt, Joint Bone Spine, 2000, 67, 366-383; Yoshida et al., Histol. Histopathol., 1999, 14, 1287-1294).

### Endothelial Tube Formation Assay as a Measure of Angiogenesis

Angiogenesis is stimulated by numerous factors that promote interaction of endothelial cells with each other and with extracellular matrix molecules, resulting in the formation of capillary tubes. This morphogenic process is necessary for the delivery of oxygen to nearby tissues and plays an essential role in embryonic development, wound healing, and tumor growth (Carmeliet and Jain, Nature, 2000, 407, 249-257). Moreover, this process can be reproduced in a tissue culture assay that evaluated the formation of tube-like structures by endothelial cells. There are several different variations of the assay that use different matrices, such as collagen I (Kanayasu et al., Lipids, 1991, 26, 271-276), Matrigel (Yamagishi et al., J. Biol. Chem., 1997, 272, 8723-8730) and fibrin (Bach et al., Exp. Cell Res., 1998, 238, 324-334), as growth substrates for the cells. In this assay, HUVECs are plated on a matrix derived from the Engelbreth-Holm-Swarm mouse tumor, which is very similar to Matrigel (Kleinman et al., Biochemistry, 1986, 25, 312-318; Madri and Pratt, J. Histochem. Cytochem., 1986, 34, 85-91). Untreated HUVECs form tube-like structures when grown on this substrate. Loss of tube formation in vitro has been correlated with the inhibition of angiogenesis in vivo (Carmeliet and Jain, Nature, 2000, 407, 249-257; Zhang et al., Cancer Res., 2002, 62, 2034-2042), which supports the use of in vitro tube formation as an endpoint for angiogenesis.

In a further embodiment, primary human umbilical vein endothelial cells (HuVECs) were used to measure the effects of antisense inhibition of apolipoprotein B on tube formation activity. HuVECs were routinely cultured in EGM® (Clonetics Corporation, Walkersville, Md.) supplemented with SINGLEQUOTS® supplements (Clonetics Corporation, Walkersville, Md.). Cells were routinely passaged by trypsinization and dilution when they reached approximately 90% confluence and were maintained for up to 15 passages. HuVECs are plated at approximately 3000 cells/well in 96-well plates. One day later, cells are transfected with antisense oligonucleotides. The tube formation assay is performed using an in vitro Angiogenesis Assay Kit (Chemicon International, Temecula, Calif.).

HUVECs were treated with ISIS147788 (SEQ ID NO: 20) to inhibit apolipoprotein B expression. An oligonucleotide with a randomized sequence, ISIS 29848 (NNNNNNNNNNNNNNNNNNNN; where N is A,T,C or G; herein incorporated as SEQ ID NO: 21) served as a negative control, a compound that does not affect tube formation. ISIS 25237 (GCCCATTGCTGGACATGC, SEQ ID NO: 25), an oligomeric compound targeted to integrin β3 (ISIS 25237) known to inhibit angiogenesis, was used as a positive control. ISIS 25237 is a chimeric oligonucleotide (“gapmers”) 18 nucleotides in length, composed of a central “gap” region consisting of ten 2′-deoxynucleotides, which is flanked on both sides (5′ and 3′ directions) by four-nucleotide “wings”. The wings are composed of 2′-O-methoxyethyl (2′-MOE) nucleotides. The internucleoside (backbone) linkages are phosphorothioate (P═S) throughout the oligonucleotides. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 75 nM of oligonucleotide and 2.25 μg/mL LIPOFECTIN®. Before adding to cells, the oligonucleotide, LIPOFECTIN® and OPTI-MEM® 1 were mixed thoroughly and incubated for 0.5 hrs. Untreated control cells received LIPOFECTIN® only. The medium was removed from the plates and the plates were tapped on sterile gauze. Each well was washed in 150 μl of phosphate-buffered saline. The wash buffer in each well was replaced with 100 μL of the oligonucleotide/OPTI-MEM® 1/LIPOFECTIN® cocktail. ISIS147788 was tested in triplicate, and the ISIS 29848 was tested in up to six replicates. The plates were incubated for approximately 4 hours at 37° C., after which the medium was removed and the plate was tapped on sterile gauze. 100 μl of full growth medium was added to each well. Approximately 50 hours after transfection, cells are transferred to 96-well plates coated with ECMATRIX® (Chemicon Inter-national). Under these conditions, untreated HUVECs form tube-like structures. After an overnight incubation at 37° C., treated and untreated cells are inspected by light microscopy. Individual wells are assigned discrete scores from 1 to 5 depending on the extent of tube formation. A score of 1 refers to a well with no tube formation while a score of 5 is given to wells where all cells are forming an extensive tubular network. Results are expressed relative to untreated control samples. Following treatment with ISIS147788, ISIS 25237 and ISIS 29848, tube formation was 100%, 40% and 100% relative to tube formation in untreated control samples. ISIS147788 did not significantly inhibit tube formation by HUVECs.

### Matrix Metalloproteinase Activity

In a further embodiment, the antisense inhibition of apolipoprotein B was evaluated for effects on MMP activity in the media above human umbilical-vein endothelial cells (HUVECs). MMP activity was measured using the ENZCHEK® Gelatinase/Collagenase Assay Kit (Molecular Probes, Eugene, Oreg.). HUVECs are cultured as described for the tube formation assay. HUVECs are plated at approximately 4000 cells per well in 96-well plates and transfected one day later.

HUVECs were treated with ISIS147788 (SEQ ID NO: 20) to inhibit apolipoprotein B mRNA expression. An oligonucleotide with a randomized sequence, ISIS 29848 (NNNNNNNNNNNNNNNNNNNN; where N is A,T,C or G; herein incorporated as SEQ ID NO: 21) served as a negative control, or a treatment not expected to affect MMP activity. ISIS 25237 (GCCCATTGCTGGACATGC, SEQ ID NO: 25) targets integrin beta 3 and was used as a positive control for the inhibition of MMP activity.

Cells were treated as described for the tube formation assay, with 75 nM of oligonucleotide and 2.25 μg/mL LIPOFECTIN®. ISIS147788 and ISIS 25237 were tested in triplicate, and the ISIS 29848 was tested in up to six replicates. The plates were incubated for approximately 4 hours at 37° C., after which the medium was removed and the plate was tapped on sterile gauze. 100 μl of full growth medium was added to each well. Approximately 50 hours after transfection, a p-aminophenylmercuric acetate (APMA, Sigma-Aldrich, St. Louis, Mo.) solution is added to each well of a Corning-Costar 96-well clear bottom plate (VWR International, Brisbane, Calif.). The APMA solution is used to promote cleavage of inactive MMP precursor proteins. Media above the HUVECs is then transferred to the wells in the 96-well plate. After 30 minutes, the quenched, fluorogenic MMP cleavage substrate is added, and baseline fluorescence is read immediately at 485 nm excitation/530 nm emission. Following an overnight incubation at 37° C. in the dark, plates are read again to determine the amount of fluorescence, which corresponds to MMP activity. Total protein from HUVEC lysates is used to normalize the readings, and MMP activites are expressed as a percent relative to MMP activity from untreated control cells that did not receive oligonucleotide treatment. MMP activities were 39%, 49% and 84% in the culture media from cells treated with ISIS147788, ISIS 25237 and ISIS 29848. These data reveal that ISIS147788, like the positive control 25237, can inhibit MMP activity and is a candidate therapeutic agent for the inhibition of angiogenesis where such activity is desired, for example, in the treatment of cancer, diabetic retinopathy, cardiovascular disease, rheumatoid arthritis and psoriasis.

### Metabolism Assays

Insulin is an essential signaling molecule throughout the body, but its major target organs are the liver, skeletal muscle and adipose tissue. Insulin is the primary modulator of glucose homeostasis and helps maintain a balance of peripheral glucose utilization and hepatic glucose production. The reduced ability of normal circulating concentrations of insulin to maintain glucose homeostasis manifests in insulin resistance which is often associated with diabetes, central obesity, hypertension, polycystic ovarian syndrom, dyslipidemia and atherosclerosis (Saltiel, Cell, 2001, 104, 517-529; Saltiel and Kahn, Nature, 2001, 414, 799-806).

### Response of Undifferentiated Adipocytes to Insulin

Insulin promotes the differentiation of preadipocytes into adipocytes. The condition of obesity, which results in increases in fat cell number, occurs even in insulin-resistant states in which glucose transport is impaired due to the antilipolytic effect of insulin. Inhibition of triglyceride breakdown requires much lower insulin concentrations than stimulation of glucose transport, resulting in maintenance or expansion of adipose stores (Kitamura et al., Mol. Cell. Biol., 1999, 19, 6286-6296; Kitamura et al., Mol. Cell. Biol., 1998, 18, 3708-3717).

One of the hallmarks of cellular differentiation is the upregulation of gene expression. During adipocyte differentiation, the gene expression patterns in adipocytes change considerably. Some genes known to be upregulated during adipocyte differentiation include hormone-sensitive lipase (HSL), adipocyte lipid binding protein (aP2), glucose transporter 4 (Glut4), and peroxisome proliferator-activated receptor gamma (PPAR-γ). Insulin signaling is improved by compounds that bind and inactivate PPAR-γ, a key regulator of adipocyte differentiation (Olefsky, J. Clin. Invest., 2000, 106, 467-472). Insulin induces the translocation of GLUT4 to the adipocyte cell surface, where it transports glucose into the cell, an activity necessary for triglyceride synthesis. In all forms of obesity and diabetes, a major factor contributing to the impaired insulin-stimulated glucose transport in adipocytes is the downregulation of GLUT4. Insulin also induces hormone sensitive lipase (HSL), which is the predominant lipase in adipocytes that functions to promote fatty acid synthesis and lipogenesis (Fredrikson et al., J. Biol. Chem., 1981, 256, 6311-6320). Adipocyte fatty acid binding protein (aP2) belongs to a multi-gene family of fatty acid and retinoid transport proteins. aP2 is postulated to serve as a lipid shuttle, solubilizing hydrophobic fatty acids and delivering them to the appropriate metabolic system for utilization (Fu et al., J. Lipid Res., 2000, 41, 2017-2023; Pelton et al., Biochem. Biophys. Res. Commun., 1999, 261, 456-458). Together, these genes play important roles in the uptake of glucose and the metabolism and utilization of fats.

Leptin secretion and an increase in triglyceride content are also well-established markers of adipocyte differentiation. While it serves as a marker for differentiated adipocytes, leptin also regulates glucose homeostasis through mechanisms (autocrine, paracrine, endocrine and neural) independent of the adipocyte's role in energy storage and release. As adipocytes differentiate, insulin increases triglyceride accumulation by both promoting triglyceride synthesis and inhibiting triglyceride breakdown (Spiegelman and Flier, Cell, 2001, 104, 531-543). As triglyceride accumulation correlates tightly with cell size and cell number, it is an excellent indicator of differentiated adipocytes.

The effects of antisense inhibition of apolipoprotein B on the expression of markers of cellular differentiation were examined in preadipocytes. Human white preadipocytes (Zen-Bio Inc., Research Triangle Park, N.C.) were grown in preadipocyte media (ZenBio Inc., Research Triangle Park, N.C.). One day before transfection, 96-well plates were seeded with approximately 3000 cells/well.

Cells were treated with ISIS147788 (SEQ ID NO: 20) to inhibit apolipoprotein B expression. An oligonucleotide with a randomized sequence, ISIS 29848 (NNNNNNNNNNNNNNNNNNNN; where N is A,T,C or G; herein incorporated as SEQ ID NO: 25) was used a negative control, a compound that does not modulate adipocyte differentiation. Tumor necrosis factor alpha (TNF-α), which inhibits adipocyte differentiation, was used as a positive control for the inhibition of adipocyte differentiation as evaluated by leptin secretion. For all other parameters measured, ISIS105990 (AGCAAAAGATCAATCCGTTA, incorporated herein as SEQ ID NO: 26), an inhibitor of PPAR-γ, served as a positive control for the inhibition of adipocyte differentiation. ISIS 29848 and ISIS105990 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 250 nM of oligonucleotide and 6.5 μg/mL LIPOFECTIN®. Before adding to cells, the oligonucleotide, LIPOFECTIN® and OPTI-MEM® 1 were mixed thoroughly and incubated for 0.5 hrs. Untreated control cells received LIPOFECTIN® only. The medium was removed from the plates and the plates were tapped on sterile gauze. Each well was washed in 150 μl of phosphate-buffered saline. The wash buffer in each well was replaced with 100 μl, of the oligonucleotide/OPTI-MEM®/LIPOFECTIN® cocktail. Compounds of the invention and ISIS105990 were tested in triplicate, ISIS 29848 was tested in up to six replicate wells. The plates were incubated for approximately 4 hours at 37° C., after which the medium was removed and the plate was tapped on sterile gauze. 100 μl of full growth medium was added to each well. After the cells have reached confluence (approximately three days), they were exposed for three days to differentiation media (Zen-Bio, Inc.) containing a PPAR-γ agonist, IBMX, dexamethasone, and insulin. Cells were then fed adipocyte media (Zen-Bio, Inc.), which was replaced at 2 to 3 day intervals. Leptin secretion into the media in which adipocytes are cultured was measured by protein ELISA. On day nine post-transfection, 96-well plates were coated with a monoclonal antibody to human leptin (R&D Systems, Minneapolis, Minn.) and left at 4° C. overnight. The plates were blocked with bovine serum albumin (BSA), and a dilution of the treated adipoctye media was incubated in the plate at room temperature for approximately 2 hours. After washing to remove unbound components, a second monoclonal antibody to human leptin (conjugated with biotin) was added. The plate was then incubated with strepavidin-conjugated horse radish peroxidase (HRP) and enzyme levels were determined by incubation with 3, 3′, 5, 5′-tetramethlybenzidine, which turns blue when cleaved by HRP. The OD₄₅₀ was read for each well, where the dye absorbance is proportional to the leptin concentration in the cell lysate. Results, shown in Table 58, are expressed as a percent control relative to untreated control samples. With respect to leptin secretion, values above or below 100% are considered to indicate that the compound has the ability to stimulate or inhibit leptin secretion, respectively.

The triglyceride accumulation assay measures the synthesis of triglyceride by adipocytes. Triglyceride accumulation is measured using the INFINITY® Triglyceride reagent kit (Sigma-Aldrich, St. Louis, Mo.). On day nine post-transfection, cells are washed and lysed at room temperature, and the triglyceride assay reagent is added. Triglyceride accumulation is measured based on the amount of glycerol liberated from triglycerides by the enzyme lipoprotein lipase. Liberated glycerol is phosphorylated by glycerol kinase, and hydrogen peroxide is generated during the oxidation of glycerol-1-phosphate to dihydroxyacetone phosphate by glycerol phosphate oxidase. Horseradish peroxidase (HRP) uses H₂O₂ to oxidize 4-aminoantipyrine and 3,5 dichloro-2-hydroxybenzene sulfonate to produce a red-colored dye. Dye absorbance, which is proportional to the concentration of glycerol, is measured at 515 nm using an UV spectrophotometer. Glycerol concentration is calculated from a standard curve for each assay, and data are normalized to total cellular protein as determined by a Bradford assay (Bio-Rad Laboratories, Hercules, Calif.). Expression of the four hallmark genes, HSL, aP2, Glut4, and PPARγ, was also measured in adipocytes transfected with compounds of the invention. Cells were lysed on day nine post-transfection, in a guanadinium-containing buffer and total RNA is harvested. The amount of total RNA in each sample was determined using a RIBOGREEN® Assay (Invitrogen Life Technologies, Carlsbad, Calif.). Real-time PCR was performed on the total RNA using primer/probe sets for the adipocyte differentiation hallmark genes Glut4, HSL, aP2, and PPAR-γ. mRNA levels, shown in Table 11, are expressed as percent control relative to the untreated control values. With respect to the four adipocyte differentiation hallmark genes, values above or below 100% are considered to indicate that the compound has the ability to stimulate or inhibit adipocyte differentiation, respectively.

TABLE 11
Effects of antisense inhibition of Apolipoprotein
B on adipocyte differentiation

| Treatment | Target | Leptin | aP2 | Glut4 | HSL | PPARγ |
|---|---|---|---|---|---|---|
| ISIS 147788 | apolipoprotein B | 137 | 99 | 101 | 74 | 149 |
| ISIS 29848 | negative control | 106 | 95 | 85 | 75 | 96 |
| ISIS 105990 | positive control | N.D. | 55 | 58 | 49 | 38 |
| TNF-alpha | positive control | 30 | N.D. | N.D. | N.D. | N.D. |

ISIS147788 resulted in an increase in leptin secretion, indicating that this compound is potentially useful for the treatment of obesity. PPAR-γ mRNA expression was also increased.

### Inflammation Assays

Inflammation assays are designed to identify genes that regulate the activation and effector phases of the adaptive immune response. During the activation phase, T lymphocytes (also known as T-cells) receiving signals from the appropriate antigens undergo clonal expansion, secrete cytokines, and upregulate their receptors for soluble growth factors, cytokines and co-stimulatory molecules (Cantrell, Annu. Rev. Immunol., 1996, 14, 259-274). These changes drive T-cell differentiation and effector function. In the effector phase, response to cytokines by non-immune effector cells controls the production of inflammatory mediators that can do extensive damage to host tissues. The cells of the adaptive immune systems, their products, as well as their interactions with various enzyme cascades involved in inflammation (e.g., the complement, clotting, fibrinolytic and kinin cascades) all represent potential points for intervention in inflammatory disease. The inflammation assay presented here measures hallmarks of the activation phase of the immune response.

Dendritic cells treated with antisense compounds are used to identify regulators of dendritic cell-mediated T-cell costimulation. The level of interleukin-2 (IL-2) production by T-cells, a critical consequence of T-cell activation (DeSilva et al., J. Immunol., 1991, 147, 3261-3267; Salomon and Bluestone, Annu. Rev. Immunol., 2001, 19, 225-252), is used as an endpoint for T-cell activation. T lymphocytes are important immunoregulatory cells that mediate pathological inflammatory responses. Optimal activation of T lymphocytes requires both primary antigen recognition events as well as secondary or costimulatory signals from antigen presenting cells (APC). Dendritic cells are the most efficient APCs known and are principally responsible for antigen presentation to T-cells, expression of high levels of costimulatory molecules during infection and disease, and the induction and maintenance of immunological memory (Banchereau and Steinman, Nature, 1998, 392, 245-252). While a number of costimulatory ligand-receptor pairs have been shown to influence T-cell activation, a principal signal is delivered by engagement of CD28 on T-cells by CD80 (B7-1) and CD86 (B7-2) on APCs (Boussiotis et al., Curr. Opin. Immunol., 1994, 6, 797-807; Lenschow et al., Annu. Rev. Immunol., 1996, 14, 233-258). Inhibition of T-cell co-stimulation by APCs holds promise for novel and more specific strategies of immune suppression. In addition, blocking costimulatory signals may lead to the development of long-term immunological anergy (unresponsiveness or tolerance) that would offer utility for promoting transplantation or dampening autoimmunity. T-cell anergy is the direct consequence of failure of T-cells to produce the growth factor IL-2 (DeSilva et al., J. Immunol., 1991, 147, 3261-3267; Salomon and Bluestone, Annu. Rev. Immunol., 2001, 19, 225-252).

### Dendritic Cell Cytokine Production as a Measure of the Activation Phase of the Immune Response

In a further embodiment of the present invention, the effect of ISIS147788 (SEQ ID NO: 20) was examined on the dendritic cell-mediated costimulation of T-cells. Dendritic cells (DCs, Clonetics Corp., San Diego, Calif.) were plated at approximately 6500 cells/well on anti-CD3 (UCHT1, Pharmingen-BD, San Diego, Calif.) coated 96-well plates in 500 U/mL granulocyte macrophase-colony stimulation factor (GM-CSF) and interleukin-4 (IL-4). DCs were treated with antisense compounds approximately 24 hours after plating.

Cells were treated with ISIS147788 (SEQ ID NO: 20) to inhibit apolipoprotein B expression. An oligonucleotide with a randomized sequence, ISIS 29848 (NNNNNNNNNNNNNNNNNNNN; where N is A,T,C or G; herein incorporated as SEQ ID NO: 21) served as a negative control, a compound that does not affect dendritic cell-mediated T-cell costimulation. ISIS113131 (CGTGTGTCTGTGCTAGTCCC, incorporated herein as SEQ ID NO: 27), an inhibitor of CD86, served as a positive control for the inhibition of dendritic cell-mediated T-cell costimulation. ISIS 29848 and ISIS113131 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Oligonucleotide was mixed with LIPOFECTIN® (Invitrogen Life Technologies, Carlsbad, Calif.) in OPTI-MEM® 1 (Invitrogen Life Technologies, Carlsbad, Calif.) to achieve a final concentration of 200 nM of oligonucleotide and 6 μg/mL LIPOFECTIN®. Before adding to cells, the oligonucleotide, LIPOFECTIN® and OPTI-MEM® 1 were mixed thoroughly and incubated for 0.5 hrs. The medium was removed from the cells and the plates were tapped on sterile gauze. Each well was washed in 150 μL of phosphate-buffered saline. The wash buffer in each well was replaced with 100 pt of the oligonucleotide/OPTI-MEM® 1/LIPOFECTIN® cocktail. Untreated control cells received LIPOFECTIN® only. ISIS147788 and ISIS113131 were tested in triplicate, and the negative control oligonucleotide was tested in up to six replicates. The plates were incubated with oligonucleotide for approximately 4 hours at 37° C., after which the medium was removed and the plate was tapped on sterile gauze. Fresh growth media plus cytokines was added and DC culture was continued for an additional 48 hours. DCs are then co-cultured with Jurkat T-cells in RPMI medium (Invitrogen Life Technologies, Carlsbad, Calif.) supplemented with 10% heat-inactivated fetal bovine serum (Sigma Chemical Company, St. Louis, Mo.). Culture supernatants are collected approximately 24 hours later and assayed for IL-2 levels (IL-2 DuoSet, R&D Systems, Minneapolis, Minn.), which are expressed as a percent relative to untreated control samples. A value greater than 100% indicates an induction of the inflammatory response, whereas a value less than 100% demonstrates a reduction in the inflammatory response.

The culture supernatant of cells treated with ISIS147788, ISIS113131 and ISIS 29848 contained IL-2 at 51%, 50% and 91% of the IL-2 concentration found in culture supernatant from untreated control cells, respectively. These results indicate that ISIS147788 inhibited T-cell co-stimulation and reduced the inflammatory response. As such, antisense oligonucleotides targeting apolipoprotein B are candidate therapeutic compounds with applications in the prevention, treatment or attenuation of conditions associated with hyperstimulation of the immune system, including rheumatoid arthritis, irritable bowel disease, athsma, lupus and multiple sclerosis.

### Example 20

### Compounds Useful for the Improvement of Cardiovascular Risk Profiles

Research from experimental animals, laboratory investigations, epidemiology, and genetic forms of hypercholesterolemia indicate that elevated LDL cholesterol (LDL-C) is a major cause of coronary heart disease (CHD). In addition, recent clinical trials robustly show that LDL-lowering therapy reduces risk for CHD. For these reasons, the NCEP Adult Treatment Panel III (ATP III) guidelines identify elevated LDL-cholesterol as the primary target of cholesterol-lowering therapy. Despite the availability of lipid-lowering therapeutic agents, only approximately 20% of high-risk patients with coronary heart disease attain the aggressive LDL-cholesterol levels recommended by the United States National Cholesterol Education Program (NCEP) Guidelines (Adult Treatment Panel III, Circulation, 2002, 106, 3143-3421). Thus, there exists a need for additional safe and effective lipid-lowering agents.

Antisense inhibition of apolipoprotein B reduces liver and serum apolipoprotein B and lowers serum LDL-cholesterol, as evidenced by studies in multiple animal models (as described in U.S. patent application Ser. No. 10/712,795, which is herein incorporated by reference in its entirety). Thus, antisense inhibition of apolipoprotein B accomplishes the cholesterol-lowering effects suggested by the NCEP. Furthermore, as described herein, antisense inhibition of apolipoprotein B shifts the gene expression profile of a high-fat fed mouse from that of an obese animal to that of a lean animal. This shift in gene expression profile provides a means for the identification of antisense compounds, including those targeted to apolipoprotein B, that are candidate lipid-lowering agents. Compounds that shift gene expression patterns from high-fat fed profiles to lean profiles are candidate therapeutic agents for the treatment of conditions such as cardiovascular disease and hyperlipidemia.

### Example 21

### Design and Screening of Duplexed Oligomeric Compounds Targeting Apolipoprotein B

In a further embodiment, a series of duplexes, including dsRNA (or siRNAs) and mimetics thereof, comprising oligomeric compounds targeted to apolipoprotein B and their complements can be designed. The nucleobase sequence of the antisense strand of the duplex comprises at least a portion of an oligonucleotide targeted to apolipoprotein B. The ends of the strands may be modified by the addition of one or more natural or modified nucleobases to form an overhang. The sense strand of the nucleic acid duplex is then designed and synthesized as the complement of the antisense strand and may also contain modifications or additions to either terminus. The antisense and sense strands of the duplex comprise from about 17 to 25 nucleotides, or from about 19 to 23 nucleotides. Alternatively, the antisense and sense strands comprise 20, 21 or 22 nucleotides.

In one embodiment, a duplex comprising an antisense strand having the sequence CGAGAGGCGGACGGGACCG (SEQ ID NO: 28), can be prepared with blunt ends (no single stranded overhang) as shown:

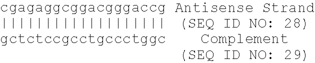

In another embodiment, both strands of the dsRNA duplex would be complementary over the central nucleobases, each having overhangs at one or both termini. For example, a duplex comprising an antisense strand having the sequence CGAGAGGCGGACGGGACCG (SEQ ID NO: 28) and having a two-nucleobase overhang of deoxythymidine(dT) would have the following structure:

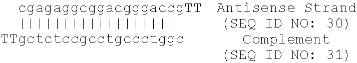

Overhangs can range from 2 to 6 nucleobases and these nucleobases may or may not be complementary to the target nucleic acid. In another embodiment, the duplexes can have an overhang on only one terminus.

The RNA duplex can be unimolecular or bimolecular; i.e, the two strands can be part of a single molecule or may be separate molecules.

RNA strands of the duplex can be synthesized by methods routine to the skilled artisan or purchased from Dharmacon Research Inc. (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM.

Once prepared, the duplexed compounds are evaluated for their ability to modulate apolipoprotein B. When cells reach approximately 80% confluency, they are treated with duplexed compounds of the invention. For cells grown in 96-well plates, wells are washed once with 200 μL OPTI-MEM® 1 reduced-serum medium (Invitrogen Life Technologies, Carlsbad, Calif.) and then treated with 130 μl, of OPTI-MEM® 1 containing 12 μg/mL LIPOFECTIN® (Invitrogen Life Technologies, Carlsbad, Calif.) and the desired duplex antisense compound (e.g. 200 nM) at a ratio of 6 μg/mL LIPOFECTIN® per 100 nM duplex antisense compound. After approximately 5 hours of treatment, the medium is replaced with fresh medium. Cells are harvested approximately 16 hours after treatment, at which time RNA is isolated and target reduction measured by real-time PCR.

What is claimed is:

1. A method comprising contacting an animal with an antisense oligonucleotide 15-30 nucleobases in length, and modulating the level of a target gene mRNA,
wherein said antisense oligonucleotide reduces the level of apolipoprotein B mRNA and wherein said target gene is selected from the group consisting of Lcat, Lip1, Lipc, Ppara, Pparg, Pcx, Apoa4, Apoc1, Apoc2, Apoc4, Mttp, Prkaa1, Prkaa2, Prkab1, Prkag1, Srebp-1, Scd2, Scd1, Acad1, Acadm, Acads, Acox1, Cpt1a, Cpt2, Crat, Elovl2, Elovl3, Acadsb, Fads2, Fasn, Facl2, Facl4, Abcd2, Dbi, Fabp1, Fabp2, Fabp7, Acat-1, Acca-1, Cyp7a1, Cyp7b1, Soat2, Ldlr, Hmgcs1, Hmgcs2, Car5a, Gck, Gck and G6 pc.

2. The method of claim 1 which results in a shift a gene expression profile of an obese animal to that of a lean animal.

3. The method of claim 1 wherein the target gene mRNA is reduced in a time dependent manner.

4. The method of claim 3 wherein the target gene mRNA is reduced in a dose dependent manner.

5. The method of claim 1 wherein said antisense oligonucleotide comprises a chimeric oligonucleotide.

6. The method of claim 1 wherein said antisense oligonucleotide has at least one modified internucleoside linkage, sugar moiety or nucleobase.

7. The method of claim 1 wherein said antisense oligonucleotide has at least one 2′-O-methoxyethyl sugar moiety.

8. The method of claim 1 wherein said antisense oligonucleotide has at least one phosphorothioate internucleoside linkage.

9. The method of claim 1 wherein at least one cytosine in said antisense oligonucleotide is a 5-methyl cytosine.

10. An antisense oligonucleotide 15-30 nucleobases in length targeted to a nucleic acid encoding apolipoprotein B that shifts a liver gene expression profile of an obese animal to that of a lean animal.

11. A method of lowering the cardiovascular risk profile of an individual, said individual having a high cardiovascular risk profile as defined by ATP III, comprising administering to said individual the compound of claim 10.

12. A method of altering a cellular pathway or metabolic process comprising contacting a cell with an antisense oligonucleotide that specifically hybridizes to and inhibits the expression of a nucleic acid molecule encoding apolipoprotein B, wherein the cellular pathway or metabolic process is apoptosis, angiogenesis, leptic secretion or T-cell co-stimulation.

13. The method of claim 12, wherein the antisense oligonucleotide comprises SEQ ID NO: 20.

14. The method of claim 12, wherein apoptosis is induced in said cells.

15. The method of claim 14 wherein said cells are cancer cells.

16. The method of claim 15 wherein said cancer cells are breast cancer cells.

17. The method of claim 12 wherein angiogenesis is inhibited.

18. The method of claim 12 wherein leptin secretion is increased.

19. The method of claim 12 wherein T-cell co-stimulation is inhibited.


SEQUENCE LISTING (854 sequences)

SEQ ID NO: 1 - DNA, 20 bp, Artificial Sequence
  Antisense Oligonucleotide
tccgtcatcgctcctcaggg

SEQ ID NO: 2 - DNA, 20 bp, Artificial Sequence
  Antisense Oligonucleotide
atgcattctgcccccaagga

SEQ ID NO: 3 - DNA, 14121 bp, H. sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaacaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctcaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagatctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatgattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggaggcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccagattagagctggaactgaggcctacaggagagattgagcagtattctgtcagcgcaacctatgagctccagagagaggacagagccttggtggataccctgaagtttgtaactcaagcagaaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatgatgaagagaagattgaatttgaatggaacacaggcaccaatgtagataccaaaaaaatgacttccaatttccctgtggatctctccgattatcctaagagcttgcatatgtatgctaatagactcctggatcacagagtccctgaaacagacatgactttccggcacgtgggttccaaattaatagttgcaatgagctcatggcttcagaaggcatctgggagtcttccttatacccagactttgcaagaccacctcaatagcctgaaggagttcaacctccagaacatgggattgccagacttccacatcccagaaaacctcttcttaaaaagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtagtggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgatgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttgacccaccaatacaagttgaaggagactattcagaagctaagcaatgtcctacaacaagttaagataaaagattactttgagaaattggttggatttattgatgatgctgtgaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagactctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtcgaaatgaaagtaaagatcatcagaaccattgaccagatgcagaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccacatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaaaaaccacaaaagccgaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattactcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatgttttgggaacacacaaaatcgaagatggtacgttagcctctaagactaaaggaacacttgcacaccgtgacttcagtgcagaatatgaagaagatggcaaatttgaaggacttcaggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaaaaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcaatgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 4 - DNA, 21 bp, Artificial Sequence
  PCR Primer
tgctaaaggcacatatggcct

SEQ ID NO: 5 - DNA, 23 bp, Artificial Sequence
  PCR Primer
ctcaggttggactctccattgag

SEQ ID NO: 6 - DNA, 28 bp, Artificial Sequence
  PCR Probe
cttgtcagagggatcctaacactggccg

SEQ ID NO: 7 - DNA, 19 bp, Artificial Sequence
  PCR Primer
gaaggtgaaggtcggagtc

SEQ ID NO: 8 - DNA, 20 bp, Artificial Sequence
  PCR Primer
gaagatggtgatgggatttc

SEQ ID NO: 9 - DNA, 20 bp, Artificial Sequence
  PCR Probe
caagcttcccgttctcagcc

SEQ ID NO: 10 - DNA, 2354 bp, M. musculus
gaattccaacttcctcacctctcacatacaattgaaatacctgcttttggcaaactgcatagcatccttaagatccaatctcctctctttatattagatgctaatgccaacatacagaatgtaacaacttcagggaacaaagcagagattgtggcttctgtcactgctaaaggagagtcccaatttgaagctctcaattttgattttcaagcacaagctcaattcctggagttaaatcctcatcctccagtcctgaaggaatccatgaacttctccagtaagcatgtgagaatggagcatgagggtgagatagtatttgatggaaaggccattgaggggaaatcagacacagtcgcaagtttacacacagagaaaaatgaagtagagtttaataatggtatgactgtcaaagtaaacaatcagctcacccttgacagtcacacaaagtacttccacaagttgagtgttcctaggctggacttctccagtaaggcttctcttaataatgaaatcaagacactattagaagctggacatgtggcattgacatcttcagggacagggtcatggaactgggcctgtcccaacttctcggatgaaggcatacattcgtcccaaattagctttactgtggatggtcccattgcttttgttggactatccaataacataaatggcaaacacttacgggtcatccaaaaactgacttatgaatctggcttcctcaactattctaagtttgaagttgagtcaaaagttgaatctcagcacgtgggctccagcattctaacagccaatggtcgggcactgctcaaggacgcaaaggcagaaatgactggtgagcacaatgccaacttaaatggaaaagttattggaactttgaaaaattctctcttcttttcagcacaaccatttgagattactgcatccacaaataatgaaggaaatttgaaagtgggttttccactaaagctgactgggaaaatagacttcctgaataactatgcattgtttctgagtccccgtgcccaacaagcaagctggcaagcgagtaccagattcaatcagtacaaatacaatcaaaacttttctgctataaacaatgaacacaacatagaagccagtataggaatgaatggagatgccaacctggatttcttaaacatacctttaacaattcctgaaattaacttgccttacacggagttcaaaactcccttactgaaggatttctccatatgggaagaaacaggcttgaaagaatttttgaagacaacaaagcaatcatttgatttgagtgtaaaggctcaatataaaaagaacagtgacaagcattccattgttgtccctctgggtatgttttatgaatttattctcaacaatgtcaattcgtgggacagaaaatttgagaaagtcagaaacaatgctttacattttcttaccacctcctataatgaagcaaaaattaaggttgataagtacaaaactgaaaattcccttaatcagccctctgggacctttcaaaatcatggctacactatcccagttgtcaacattgaagtatctccatttgctgtagagacactggcttccaggcatgtgatccccacagcaataagcaccccaagtgtcacaatccctggtcctaacatcatggtgccttcatacaagttagtgctgccacccctggagttgccagttttccatggtcctgggaatctattcaagtttttcctcccagatttcaagggattcaacactattgacaatatttatattccagccatgggcaactttacctatgacttttcttttaaatcaagtgtcatcacactgaataccaatgctggactttataaccaatcagatatcgttgcccatttcctttcttcctcttcatttgtcactgacgccctgcagtacaaattagagggaacatcacgtctgatgcgaaaaaggggattgaaactagccacagctgtctctctaactaacaaatttgtaaagggcagtcatgacagcaccattagtttaaccaagaaaaacatggaagcatcagtgagaacaactgccaacctccatgctcccatattctcaatgaacttcaagcaggaacttaatggaaataccaagtcaaaacccactgtttcatcatccattgaactaaactatgacttcaattcctcaaagctgcactctactgcaacaggaggcattgatcacaagttcagcttagaaagtctcacttcctacttttccattgagtcattcaccaaaggaaatatcaagagttccttcctttctcaggaatattcaggaagtgttgccaatgaagccaatgtatatctgaattc

SEQ ID NO: 11 - DNA, 19 bp, Artificial Sequence
  PCR Primer
cgtgggctccagcattcta

SEQ ID NO: 12 - DNA, 21 bp, Artificial Sequence
  PCR Primer
agtcatttctgcctttgcgtc

SEQ ID NO: 13 - DNA, 22 bp, Artificial Sequence
  PCR Probe
ccaatggtcgggcactgctcaa

SEQ ID NO: 14 - DNA, 20 bp, Artificial Sequence
  PCR Primer
ggcaaattcaacggcacagt

SEQ ID NO: 15 - DNA, 20 bp, Artificial Sequence
  PCR Primer
gggtctcgctcctggaagat

SEQ ID NO: 16 - DNA, 27 bp, Artificial Sequence
  PCR Probe
aaggccgagaatgggaagcttgtcatc

SEQ ID NO: 17 - DNA, 20 bp, Artificial Sequence
  Antisense Oligonucleotide
gtccctgaagatgtcaatgc

SEQ ID NO: 18 - DNA, 20 bp, Artificial Sequence
  Antisense Oligonucleotide
atgtcaatgccacatgtcca

SEQ ID NO: 19 - DNA, 20 bp, Artificial Sequence
  Antisense Oligonucleotide
ccttccctgaaggttcctcc

SEQ ID NO: 20 - DNA, 20 bp, Artificial Sequence
  Antisense Oligonucleotide
tttctgttgccacattgccc

SEQ ID NO: 21 - DNA, 20 bp, Artificial Sequence
  Antisense Oligonucleotide
  misc_feature (1)...(20): n = A, T, C or G
nnnnnnnnnnnnnnnnnnnn

SEQ ID NO: 22 - DNA, 20 bp, Artificial Sequence
  Antisense Oligonucleotide
atccaagtgctactgtagta

SEQ ID NO: 23 - DNA, 20 bp, Artificial Sequence
  Antisense Oligonucleotide
ttgtcccagtcccaggcctc

SEQ ID NO: 24 - DNA, 20 bp, Artificial Sequence
  Antisense Oligonucleotide
gccctccatgctggcacagg

SEQ ID NO: 25 - DNA, 18 bp, Artificial Sequence
  Antisense Oligonucleotide
gcccattgctggacatgc

SEQ ID NO: 26 - DNA, 20 bp, Artificial Sequence
  Antisense Oligonucleotide
agcaaaagatcaatccgtta

SEQ ID NO: 27 - DNA, 20 bp, Artificial Sequence
  Antisense Oligonucleotide
cgtgtgtctgtgctagtccc

SEQ ID NO: 28 - RNA, 19 nt, Artificial Sequence
  Antisense Oligonucleotide
cgagaggcggacgggaccg

SEQ ID NO: 29 - RNA, 19 nt, Artificial Sequence
  Antisense Oligonucleotide
gcucuccgccugcccuggc

SEQ ID NO: 30 - DNA, 21 bp, Artificial Sequence
  Antisense Oligonucleotide
  misc_feature (1)...(19): bases at these positions are RNA
cgagaggcggacgggaccgtt

SEQ ID NO: 31 - DNA, 21 bp, Artificial Sequence
  Antisense Oligonucleotide
  misc_feature (3)...(21): bases at these positions are RNA
ttgcucuccgccugcccuggc

SEQ ID NO: 32-126 - skipped (95 entries)

SEQ ID NO: 127 - DNA, 20 bp, Artificial Sequence
  Antisense Oligonucleotide
gggttgaagccatacacctc

SEQ ID NO: 128 - DNA, 20 bp, Artificial Sequence
  Antisense Oligonucleotide
ccagcttgagctcatacctg

SEQ ID NO: 129 - DNA, 20 bp, Artificial Sequence
  Antisense Oligonucleotide
ccctcttgatgttcaggatg

SEQ ID NO: 130 - DNA, 20 bp, Artificial Sequence
  Antisense Oligonucleotide
gagcagtttccatacacggt

SEQ ID NO: 131 - DNA, 20 bp, Artificial Sequence
  Antisense Oligonucleotide
cccttcctcgtcttgacggt

SEQ ID NO: 132 - DNA, 20 bp, Artificial Sequence
  Antisense Oligonucleotide
ttgaagcgatcacactgccc

SEQ ID NO: 133 - DNA, 20 bp, Artificial Sequence
  Antisense Oligonucleotide
gcctttgatgagagcaagtg

SEQ ID NO: 134 - DNA, 20 bp, Artificial Sequence
  Antisense Oligonucleotide
tcctcttagcgtccagtgtg

SEQ ID NO: 135 - skipped

SEQ ID NO: 136 - DNA, 20 bp, Artificial Sequence
  Antisense Oligonucleotide
gcactgaggctgtccacact

SEQ ID NO: 137 - skipped

SEQ ID NO: 138 - DNA, 20 bp, Artificial Sequence
  Antisense Oligonucleotide
gttgaccgcgtggctcagcg

SEQ ID NO: 139 - DNA, 20 bp, Artificial Sequence
  Antisense Oligonucleotide
gcagctcctgggtccctgta

SEQ ID NO: 140 - DNA, 20 bp, Artificial Sequence
  Antisense Oligonucleotide
cccatggtagaatttggaca

SEQ ID NO: 141 - DNA, 20 bp, Artificial Sequence
  Antisense Oligonucleotide
aatctcgatgaggtcagctg

SEQ ID NO: 142 - DNA, 20 bp, Artificial Sequence
  Antisense Oligonucleotide
gacaccatcaggaacttgac

SEQ ID NO: 143 - DNA, 20 bp, Artificial Sequence
  Antisense Oligonucleotide
gctcctctcccaagatgcgg

SEQ ID NO: 144 - DNA, 20 bp, Artificial Sequence
  Antisense Oligonucleotide
ggcacccatcagaagcagct

SEQ ID NO: 145 - DNA, 20 bp, Artificial Sequence
  Antisense Oligonucleotide
agtccggaatgatgatgccc

SEQ ID NO: 146 - DNA, 20 bp, Artificial Sequence
  Antisense Oligonucleotide
ctgagcagcttgactggtct

SEQ ID NO: 147 - DNA, 20 bp, Artificial Sequence
  Antisense Oligonucleotide
cccggtcagcggatagtagg

SEQ ID NO: 148 - DNA, 20 bp, Artificial Sequence
  Antisense Oligonucleotide
tgtcacaacttaggtggccc

SEQ ID NO: 149 - DNA, 20 bp, Artificial Sequence
  Antisense Oligonucleotide
gtctggcaatcccatgttct

SEQ ID NO: 150 - DNA, 20 bp, Artificial Sequence
  Antisense Oligonucleotide
cccacagacttgaagtggag

SEQ ID NO: 151 - DNA, 20 bp, Artificial Sequence
  Antisense Oligonucleotide
gaactgcccatcaatcttga

SEQ ID NO: 152 - DNA, 20 bp, Artificial Sequence
  Antisense Oligonucleotide
cccagagaggccaagctctg

SEQ ID NO: 153 - DNA, 20 bp, Artificial Sequence
  Antisense Oligonucleotide
tgtgttccctgaagcggcca

SEQ ID NO: 154 - DNA, 20 bp, Artificial Sequence
  Antisense Oligonucleotide
acccagaatcatggcctgat

SEQ ID NO: 155 - DNA, 20 bp, Artificial Sequence
  Antisense Oligonucleotide
ggtgcctgtctgctcagctg

SEQ ID NO: 156 - DNA, 20 bp, Artificial Sequence
  Antisense Oligonucleotide
atgtgaaacttgtctctccc

SEQ ID NO: 157 - DNA, 20 bp, Artificial Sequence
  Antisense Oligonucleotide
tatgtctgcagttgagatag

SEQ ID NO: 158 - DNA, 20 bp, Artificial Sequence
  Antisense Oligonucleotide
ttgaatccaggatgcagtac

SEQ ID NO: 159 - DNA, 20 bp, Artificial Sequence
  Antisense Oligonucleotide
gagtctctgagtcacctcac

SEQ ID NO: 160 - DNA, 20 bp, Artificial Sequence
  Antisense Oligonucleotide
gatagaatattgctctgcaa

SEQ ID NO: 161 - DNA, 20 bp, Artificial Sequence
  Antisense Oligonucleotide
cccttgctctaccaatgctt

SEQ ID NO: 162 - DNA, 20 bp, Artificial Sequence
  Antisense Oligonucleotide
tccattccctatgtcagcat

SEQ ID NO: 163 - DNA, 20 bp, Artificial Sequence
  Antisense Oligonucleotide
gactccttcagagccagcgg

SEQ ID NO: 164 - DNA, 20 bp, Artificial Sequence
  Antisense Oligonucleotide
cccatgctccgttctcaggt

SEQ ID NO: 165 - DNA, 20 bp, Artificial Sequence
  Antisense Oligonucleotide
cgcaggtcagcctgactaga

SEQ ID NO: 166 - DNA, 20 bp, Artificial Sequence
  Antisense Oligonucleotide
cagttagaacactgtggccc

SEQ ID NO: 167 - DNA, 20 bp, Artificial Sequence
  Antisense Oligonucleotide
cagtgtgatgacacttgatt

SEQ ID NO: 168 - DNA, 20 bp, Artificial Sequence
  Antisense Oligonucleotide
ctgtggctaacttcaatccc

SEQ ID NO: 169 - DNA, 20 bp, Artificial Sequence
  Antisense Oligonucleotide
cagtactgttatgactaccc

SEQ ID NO: 170 - DNA, 20 bp, Artificial Sequence
  Antisense Oligonucleotide
cactgaagaccgtgtgctct

SEQ ID NO: 171 - DNA, 20 bp, Artificial Sequence
  Antisense Oligonucleotide
tcgtactgtgctcccagagg

SEQ ID NO: 172 - DNA, 20 bp, Artificial Sequence
  Antisense Oligonucleotide
aagaggccctctagctgtaa

SEQ ID NO: 173 - DNA, 20 bp, Artificial Sequence
  Antisense Oligonucleotide
aagacccagaatgaatccgg

SEQ ID NO: 174 - DNA, 20 bp, Artificial Sequence
  Antisense Oligonucleotide
gtctacctcaaagcgtgcag

SEQ ID NO: 175 - skipped

SEQ ID NO: 176 - DNA, 20 bp, Artificial Sequence
  Antisense Oligonucleotide
ccatatccatgcccacggtg

SEQ ID NO: 177 - DNA, 20 bp, Artificial Sequence
  Antisense Oligonucleotide
agtttcctcatcagattccc

SEQ ID NO: 178 - DNA, 20 bp, Artificial Sequence
  Antisense Oligonucleotide
cccagtggtacttgttgaca

SEQ ID NO: 179 - DNA, 20 bp, Artificial Sequence
  Antisense Oligonucleotide
cccagtggtgccactggctg

SEQ ID NO: 180 - DNA, 20 bp, Artificial Sequence
  Antisense Oligonucleotide
gtcaacagttcctggtacag

SEQ ID NO: 181 - DNA, 20 bp, Artificial Sequence
  Antisense Oligonucleotide
ccctagtgtatatcccaggt

SEQ ID NO: 182 - DNA, 20 bp, Artificial Sequence
  Antisense Oligonucleotide
ctgaagattacgtagcacct

SEQ ID NO: 183 - DNA, 20 bp, Artificial Sequence
  Antisense Oligonucleotide
gtccagccaactatacttgg

SEQ ID NO: 184 - DNA, 20 bp, Artificial Sequence
  Antisense Oligonucleotide
cctggagcaagcttcatgta

SEQ ID NO: 185 - DNA, 20 bp, Artificial Sequence
  Antisense Oligonucleotide
tggacagaccaggctgacat

SEQ ID NO: 186 - DNA, 20 bp, Artificial Sequence
  Antisense Oligonucleotide
atgtgtacttccggaggtgc

SEQ ID NO: 187 - DNA, 20 bp, Artificial Sequence
  Antisense Oligonucleotide
tcttcaggatgaagctgcag

SEQ ID NO: 188 - DNA, 20 bp, Artificial Sequence
  Antisense Oligonucleotide
tcagcaaggctttgccctca

SEQ ID NO: 189 - DNA, 20 bp, Artificial Sequence
  Antisense Oligonucleotide
ctgcttcccttctggaatgg

SEQ ID NO: 190 - DNA, 20 bp, Artificial Sequence
  Antisense Oligonucleotide
tgccacattgcccttcctcg

SEQ ID NO: 191 - DNA, 20 bp, Artificial Sequence
  Antisense Oligonucleotide
gctgatcagagttgacaagg

SEQ ID NO: 192 - DNA, 20 bp, Artificial Sequence
  Antisense Oligonucleotide
tactgacaggactggctgct

SEQ ID NO: 193 - DNA, 20 bp, Artificial Sequence
  Antisense Oligonucleotide
gatggcttctgccacatgct

SEQ ID NO: 194 - DNA, 20 bp, Artificial Sequence
  Antisense Oligonucleotide
gatgtggatttggtgctctc

SEQ ID NO: 195 - DNA, 20 bp, Artificial Sequence
  Antisense Oligonucleotide
tgactgcttcatcactgagg

SEQ ID NO: 196 - DNA, 20 bp, Artificial Sequence
  Antisense Oligonucleotide
ggtaggtgaccacatctatc

SEQ ID NO: 197 - DNA, 20 bp, Artificial Sequence
  Antisense Oligonucleotide
tcgcagctgctgtgctgagg

SEQ ID NO: 198 - DNA, 20 bp, Artificial Sequence
  Antisense Oligonucleotide
ttccaatgacccgcagaatc

SEQ ID NO: 199 - DNA, 20 bp, Artificial Sequence
  Antisense Oligonucleotide
gatcatcagtgatggctttg

SEQ ID NO: 200 - DNA, 20 bp, Artificial Sequence
  Antisense Oligonucleotide
agcctggatggcagctttct

SEQ ID NO: 201 - DNA, 20 bp, Artificial Sequence
  Antisense Oligonucleotide
gtctgaagaagaacctcctg

SEQ ID NO: 202 - DNA, 20 bp, Artificial Sequence
  Antisense Oligonucleotide
tatctgcctgtgaaggactc

SEQ ID NO: 203 - DNA, 20 bp, Artificial Sequence
  Antisense Oligonucleotide
ctgagttcaagatattggca

SEQ ID NO: 204 - DNA, 20 bp, Artificial Sequence
  Antisense Oligonucleotide
cttccaagccaatctcgatg

SEQ ID NO: 205 - DNA, 20 bp, Artificial Sequence
  Antisense Oligonucleotide
tgcaactgtaatccagctcc

SEQ ID NO: 206 - DNA, 20 bp, Artificial Sequence
  Antisense Oligonucleotide
ccagttcagcctgcatgttg

SEQ ID NO: 207 - DNA, 20 bp, Artificial Sequence
  Antisense Oligonucleotide
gtagagaccaaatgtaatgt

SEQ ID NO: 208 - DNA, 20 bp, Artificial Sequence
  Antisense Oligonucleotide
cgttggagtaagcgcctgag

SEQ ID NO: 209 - DNA, 20 bp, Artificial Sequence
  Antisense Oligonucleotide
cagctctaatctggtgtccc

SEQ ID NO: 210 - DNA, 20 bp, Artificial Sequence
  Antisense Oligonucleotide
ctgtcctctctctggagctc

SEQ ID NO: 211 - DNA, 20 bp, Artificial Sequence
  Antisense Oligonucleotide
caaggtcatactctgccgat

SEQ ID NO: 212 - DNA, 20 bp, Artificial Sequence
  Antisense Oligonucleotide
gtatggaaataacacccttg

SEQ ID NO: 213 - DNA, 20 bp, Artificial Sequence
  Antisense Oligonucleotide
taagctgtagcagatgagtc

SEQ ID NO: 214 - DNA, 20 bp, Artificial Sequence
  Antisense Oligonucleotide
tagatctctggaggatttgc

SEQ ID NO: 215 - DNA, 20 bp, Artificial Sequence
  Antisense Oligonucleotide
gtctagaacacccaggagag

SEQ ID NO: 216 - DNA, 20 bp, Artificial Sequence
  Antisense Oligonucleotide
accacagagtcagccttcat

SEQ ID NO: 217 - DNA, 20 bp, Artificial Sequence
  Antisense Oligonucleotide
aagcagacatctgtggtccc

SEQ ID NO: 218 - DNA, 20 bp, Artificial Sequence
  Antisense Oligonucleotide
ctctccattgagccggccag

SEQ ID NO: 219 - DNA, 20 bp, Artificial Sequence
  Antisense Oligonucleotide
cctgatattcagaacgcagc

SEQ ID NO: 220 - DNA, 20 bp, Artificial Sequence
  Antisense Oligonucleotide
cagtgcctaagatgtcagca

SEQ ID NO: 221 - DNA, 20 bp, Artificial Sequence
  Antisense Oligonucleotide
agcaccaggagactacactt

SEQ ID NO: 222 - DNA, 20 bp, Artificial Sequence
  Antisense Oligonucleotide
cccatccagactgaattttg

SEQ ID NO: 223 - DNA, 20 bp, Artificial Sequence
  Antisense Oligonucleotide
ggttctagccgtagtttccc

SEQ ID NO: 224 - DNA, 20 bp, Artificial Sequence
  Antisense Oligonucleotide
aggttaccagccacatgcag

SEQ ID NO: 225 - DNA, 20 bp, Artificial Sequence
  Antisense Oligonucleotide
atgtgcatcgatggtcatgg

SEQ ID NO: 226 - DNA, 20 bp, Artificial Sequence
  Antisense Oligonucleotide
ccagagagcgagtttcccat

SEQ ID NO: 227 - DNA, 20 bp, Artificial Sequence
  Antisense Oligonucleotide
ctagacacgagatgatgact

SEQ ID NO: 228 - DNA, 20 bp, Artificial Sequence
  Antisense Oligonucleotide
tccaagtcctggctgtattc

SEQ ID NO: 229 - DNA, 20 bp, Artificial Sequence
  Antisense Oligonucleotide
cgtccagtaagctccacgcc

SEQ ID NO: 230 - DNA, 20 bp, Artificial Sequence
  Antisense Oligonucleotide
tcaacggcatctctcatctc

SEQ ID NO: 231 - DNA, 20 bp, Artificial Sequence
  Antisense Oligonucleotide
tgatagtgctcatcaagact

SEQ ID NO: 232 - DNA, 20 bp, Artificial Sequence
  Antisense Oligonucleotide
gattctgatttggtacttag

SEQ ID NO: 233 - DNA, 20 bp, Artificial Sequence
  Antisense Oligonucleotide
ctctcgattaactcatggac

SEQ ID NO: 234 - DNA, 20 bp, Artificial Sequence
  Antisense Oligonucleotide
atacactgcaactgtggcct

SEQ ID NO: 235 - DNA, 20 bp, Artificial Sequence
  Antisense Oligonucleotide
gcaagagtccaccaatcaga

SEQ ID NO: 236 - DNA, 20 bp, Artificial Sequence
  Antisense Oligonucleotide
agagcctgaagactgacttc

SEQ ID NO: 237 - DNA, 20 bp, Artificial Sequence
  Antisense Oligonucleotide
tccctcatctgagaatctgg

SEQ ID NO: 238 - DNA, 20 bp, Artificial Sequence
  Antisense Oligonucleotide
cagtgcatcaatgacagatg

SEQ ID NO: 239 - DNA, 20 bp, Artificial Sequence
  Antisense Oligonucleotide
ccgaacccttgacatctcct

SEQ ID NO: 240 - DNA, 20 bp, Artificial Sequence
  Antisense Oligonucleotide
gcctcactagcaatagttcc

SEQ ID NO: 241 - DNA, 20 bp, Artificial Sequence
  Antisense Oligonucleotide
gacatttgccatggagagag

SEQ ID NO: 242 - DNA, 20 bp, Artificial Sequence
  Antisense Oligonucleotide
ctgtctcctaccaatgctgg

SEQ ID NO: 243 - DNA, 20 bp, Artificial Sequence
  Antisense Oligonucleotide
tctgcactgaagtcacggtg

SEQ ID NO: 244 - DNA, 20 bp, Artificial Sequence
  Antisense Oligonucleotide
tcccggaccctcaactcagt

SEQ ID NO: 245 - DNA, 20 bp, Artificial Sequence
  Antisense Oligonucleotide
gcaggtccagttcatatgtg

SEQ ID NO: 246 - DNA, 20 bp, Artificial Sequence
  Antisense Oligonucleotide
gccatccttctgagttcaga

SEQ ID NO: 247 - DNA, 20 bp, Artificial Sequence
  Antisense Oligonucleotide
gcctcagtctgcttcgcacc

SEQ ID NO: 248 - DNA, 20 bp, Artificial Sequence
  Antisense Oligonucleotide
ccccgcaggtcccggtggga

SEQ ID NO: 249 - DNA, 20 bp, Artificial Sequence
  Antisense Oligonucleotide
cagccccgcaggtcccggtg

SEQ ID NO: 250 - DNA, 20 bp, Artificial Sequence
  Antisense Oligonucleotide
caaccgagaagggcactcag

SEQ ID NO: 251 - DNA, 20 bp, Artificial Sequence
  Antisense Oligonucleotide
cctcagcggcagcaaccgag

SEQ ID NO: 252 - DNA, 20 bp, Artificial Sequence
  Antisense Oligonucleotide
tcctcagcggcagcaaccga

SEQ ID NO: 253 - DNA, 20 bp, Artificial Sequence
  Antisense Oligonucleotide
ctcctcagcggcagcaaccg

SEQ ID NO: 254 - DNA, 20 bp, Artificial Sequence
  Antisense Oligonucleotide
ggctcctcagcggcagcaac

SEQ ID NO: 255 - DNA, 20 bp, Artificial Sequence
  Antisense Oligonucleotide
ggcgggctcctcagcggcag

SEQ ID NO: 256 - DNA, 20 bp, Artificial Sequence
  Antisense Oligonucleotide
ggtccatcgccagctgcggt

SEQ ID NO: 257 - DNA, 20 bp, Artificial Sequence
  Antisense Oligonucleotide
ggcgggtccatcgccagctg

SEQ ID NO: 258 - DNA, 20 bp, Artificial Sequence
  Antisense Oligonucleotide
tagaggatgatagtaagttc

SEQ ID NO: 259 - DNA, 20 bp, Artificial Sequence
  Antisense Oligonucleotide
aaatgaagatttcttttaaa

SEQ ID NO: 260 - DNA, 20 bp, Artificial Sequence
  Antisense Oligonucleotide
tatgtgaaagttcaattgga

SEQ ID NO: 261 - DNA, 20 bp, Artificial Sequence
  Antisense Oligonucleotide
atataggcagtttgaatttt

SEQ ID NO: 262 - DNA, 20 bp, Artificial Sequence
  Antisense Oligonucleotide
gctcactgtatggttttatc

SEQ ID NO: 263 - DNA, 20 bp, Artificial Sequence
  Antisense Oligonucleotide
ggctcactgtatggttttat

SEQ ID NO: 264 - DNA, 20 bp, Artificial Sequence
  Antisense Oligonucleotide
ggctggctcactgtatggtt

SEQ ID NO: 265 - DNA, 20 bp, Artificial Sequence
  Antisense Oligonucleotide
aggctggctcactgtatggt

SEQ ID NO: 266 - DNA, 20 bp, Artificial Sequence
  Antisense Oligonucleotide
aaggctggctcactgtatgg

SEQ ID NO: 267 - DNA, 20 bp, Artificial Sequence
  Antisense Oligonucleotide
ctactgcaaggctggctcac

SEQ ID NO: 268 - DNA, 20 bp, Artificial Sequence
  Antisense Oligonucleotide
actgcctactgcaaggctgg

SEQ ID NO: 269 - DNA, 20 bp, Artificial Sequence
  Antisense Oligonucleotide
tgcttatagtctactgccta

SEQ ID NO: 270 - DNA, 20 bp, Artificial Sequence
  Antisense Oligonucleotide
ttctgcttatagtctactgc

SEQ ID NO: 271 - DNA, 20 bp, Artificial Sequence
  Antisense Oligonucleotide
tttggtgcaggtccagttca

SEQ ID NO: 272 - DNA, 20 bp, Artificial Sequence
  Antisense Oligonucleotide
cagctttggtgcaggtccag

SEQ ID NO: 273 - DNA, 20 bp, Artificial Sequence
  Antisense Oligonucleotide
gccagctttggtgcaggtcc

SEQ ID NO: 274 - DNA, 20 bp, Artificial Sequence
  Antisense Oligonucleotide
tggtgccagctttggtgcag

SEQ ID NO: 275 - DNA, 20 bp, Artificial Sequence
  Antisense Oligonucleotide
gccctggtgccagctttggt

SEQ ID NO: 276 - DNA, 20 bp, Artificial Sequence
  Antisense Oligonucleotide
gagttcagagaccttccgag

SEQ ID NO: 277 - DNA, 20 bp, Artificial Sequence
  Antisense Oligonucleotide
aaatgccatccttctgagtt

SEQ ID NO: 278 - DNA, 20 bp, Artificial Sequence
  Antisense Oligonucleotide
aaaaatgccatccttctgag

SEQ ID NO: 279 - DNA, 20 bp, Artificial Sequence
  Antisense Oligonucleotide
aaaataactcagatcctgat

SEQ ID NO: 280 - DNA, 20 bp, Artificial Sequence
  Antisense Oligonucleotide
agcaaaataactcagatcct

SEQ ID NO: 281 - DNA, 20 bp, Artificial Sequence
  Antisense Oligonucleotide
agtttagcaaaataactcag

SEQ ID NO: 282 - DNA, 20 bp, Artificial Sequence
  Antisense Oligonucleotide
tcccccaagtttagcaaaat

SEQ ID NO: 283 - DNA, 20 bp, Artificial Sequence
  Antisense Oligonucleotide
ttcctcctcccccaagttta

SEQ ID NO: 284 - DNA, 20 bp, Artificial Sequence
  Antisense Oligonucleotide
agactccatttatttgttcc

SEQ ID NO: 285 - DNA, 20 bp, Artificial Sequence
  Antisense Oligonucleotide
cttctgcttgagttacaaac

SEQ ID NO: 286 - DNA, 20 bp, Artificial Sequence
  Antisense Oligonucleotide
accttctgcttgagttacaa

SEQ ID NO: 287 - DNA, 20 bp, Artificial Sequence
  Antisense Oligonucleotide
gcaccttctgcttgagttac

SEQ ID NO: 288 - DNA, 20 bp, Artificial Sequence
  Antisense Oligonucleotide
tcgcaccttctgcttgagtt

SEQ ID NO: 289 - DNA, 20 bp, Artificial Sequence
  Antisense Oligonucleotide
cttcgcaccttctgcttgag

SEQ ID NO: 290 - DNA, 20 bp, Artificial Sequence
  Antisense Oligonucleotide
tgcttcgcaccttctgcttg

SEQ ID NO: 291 - DNA, 20 bp, Artificial Sequence
  Antisense Oligonucleotide
tctgcttcgcaccttctgct

SEQ ID NO: 292 - DNA, 20 bp, Artificial Sequence
  Antisense Oligonucleotide
agtctgcttcgcaccttctg

SEQ ID NO: 293 - DNA, 20 bp, Artificial Sequence
  Antisense Oligonucleotide
tcagtctgcttcgcaccttc

SEQ ID NO: 294 - DNA, 20 bp, Artificial Sequence
  Antisense Oligonucleotide
cctcagtctgcttcgcacct

SEQ ID NO: 295 - DNA, 20 bp, Artificial Sequence
  Antisense Oligonucleotide
agcctcagtctgcttcgcac

SEQ ID NO: 296 - DNA, 20 bp, Artificial Sequence
  Antisense Oligonucleotide
gtagcctcagtctgcttcgc

SEQ ID NO: 297 - DNA, 20 bp, Artificial Sequence
  Antisense Oligonucleotide
tggtagcctcagtctgcttc

SEQ ID NO: 298 - DNA, 20 bp, Artificial Sequence
  Antisense Oligonucleotide
catggtagcctcagtctgct

SEQ ID NO: 299 - DNA, 20 bp, Artificial Sequence
  Antisense Oligonucleotide
gtcatggtagcctcagtctg

SEQ ID NO: 300 - DNA, 20 bp, Artificial Sequence
  Antisense Oligonucleotide
atgtcatggtagcctcagtc

SEQ ID NO: 301 - DNA, 20 bp, Artificial Sequence
  Antisense Oligonucleotide
gaatgtcatggtagcctcag

SEQ ID NO: 302 - DNA, 20 bp, Artificial Sequence
  Antisense Oligonucleotide
ttgaatgtcatggtagcctc

SEQ ID NO: 303 - DNA, 20 bp, Artificial Sequence
  Antisense Oligonucleotide
atttgaatgtcatggtagcc

SEQ ID NO: 304 - DNA, 20 bp, Artificial Sequence
  Antisense Oligonucleotide
atatttgaatgtcatggtag

SEQ ID NO: 305 - DNA, 20 bp, Artificial Sequence
  Antisense Oligonucleotide
cagccacatgcagcttcagg

SEQ ID NO: 306 - DNA, 20 bp, Artificial Sequence
  Antisense Oligonucleotide
accagccacatgcagcttca

SEQ ID NO: 307 - DNA, 20 bp, Artificial Sequence
  Antisense Oligonucleotide
ttaccagccacatgcagctt

SEQ ID NO: 308 - DNA, 20 bp, Artificial Sequence
  Antisense Oligonucleotide
ggttaccagccacatgcagc

SEQ ID NO: 309 - DNA, 20 bp, Artificial Sequence
  Antisense Oligonucleotide
taggttaccagccacatgca

SEQ ID NO: 310 - DNA, 20 bp, Artificial Sequence
  Antisense Oligonucleotide
tttaggttaccagccacatg

SEQ ID NO: 311 - DNA, 20 bp, Artificial Sequence
  Antisense Oligonucleotide
cttttaggttaccagccaca

SEQ ID NO: 312 - DNA, 20 bp, Artificial Sequence
  Antisense Oligonucleotide
tccttttaggttaccagcca

SEQ ID NO: 313 - DNA, 20 bp, Artificial Sequence
  Antisense Oligonucleotide
gctccttttaggttaccagc

SEQ ID NO: 314 - DNA, 20 bp, Artificial Sequence
  Antisense Oligonucleotide
aggctccttttaggttacca

SEQ ID NO: 315 - DNA, 20 bp, Artificial Sequence
  Antisense Oligonucleotide
gtaggctccttttaggttac

SEQ ID NO: 316 - DNA, 20 bp, Artificial Sequence
  Antisense Oligonucleotide
tggtaggctccttttaggtt

SEQ ID NO: 317 - DNA, 20 bp, Artificial Sequence
  Antisense Oligonucleotide
tttggtaggctccttttagg

SEQ ID NO: 318 - skipped

SEQ ID NO: 319 - DNA, 20 bp, Artificial Sequence
  Antisense Oligonucleotide
gcctcagtctgcttcgcgcc

SEQ ID NO: 320 - DNA, 20 bp, Artificial Sequence
  Antisense Oligonucleotide
gctcactgttcagcatctgg

SEQ ID NO: 321 - DNA, 20 bp, Artificial Sequence
  Antisense Oligonucleotide
tgagaatctgggcgaggccc

SEQ ID NO: 322 - skipped

SEQ ID NO: 323 - DNA, 20 bp, Artificial Sequence
  Antisense Oligonucleotide
cctccctcatgaacatagtg

SEQ ID NO: 324 - DNA, 20 bp, Artificial Sequence
  Antisense Oligonucleotide
gacgtcagaacctatgatgg

SEQ ID NO: 325 - DNA, 20 bp, Artificial Sequence
  Antisense Oligonucleotide
tgagtgagtcaatcagcttc

SEQ ID NO: 326 - DNA, 20 bp, Artificial Sequence
  Antisense Oligonucleotide
gccttctgcttgagttacaa

SEQ ID NO: 327 - DNA, 20 bp, Artificial Sequence
  Antisense Oligonucleotide
gcgccttctgcttgagttac

SEQ ID NO: 328 - DNA, 20 bp, Artificial Sequence
  Antisense Oligonucleotide
tcgcgccttctgcttgagtt

SEQ ID NO: 329 - DNA, 20 bp, Artificial Sequence
  Antisense Oligonucleotide
cttcgcgccttctgcttgag

SEQ ID NO: 330 - DNA, 20 bp, Artificial Sequence
  Antisense Oligonucleotide
agtctgcttcgcgccttctg

SEQ ID NO: 331 - DNA, 20 bp, Artificial Sequence
  Antisense Oligonucleotide
tcagtctgcttcgcgccttc

SEQ ID NO: 332 - DNA, 20 bp, Artificial Sequence
  Antisense Oligonucleotide
cctcagtctgcttcgcgcct

SEQ ID NO: 333 - DNA, 20 bp, Artificial Sequence
  Antisense Oligonucleotide
agcctcagtctgcttcgcgc

SEQ ID NO: 334 - skipped

SEQ ID NO: 335 - DNA, 20 bp, Artificial Sequence
  Antisense Oligonucleotide
tctgtaagacaggagaaaga

SEQ ID NO: 336 - DNA, 20 bp, Artificial Sequence
  Antisense Oligonucleotide
atttcctcttctgtaagaca

SEQ ID NO: 337 - DNA, 20 bp, Artificial Sequence
  Antisense Oligonucleotide
gatgccttacttggacagac

SEQ ID NO: 338 - DNA, 20 bp, Artificial Sequence
  Antisense Oligonucleotide
agaaatagctctcccaagga

SEQ ID NO: 339 - DNA, 20 bp, Artificial Sequence
  Antisense Oligonucleotide
gtcgcatcttctaacgtggg

SEQ ID NO: 340 - skipped

SEQ ID NO: 341 - DNA, 20 bp, Artificial Sequence
  Antisense Oligonucleotide
tggctcatgtctaccatatt

SEQ ID NO: 342 - DNA, 20 bp, Artificial Sequence
  Antisense Oligonucleotide
cagttgaaatgcagctaatg

SEQ ID NO: 343 - DNA, 20 bp, Artificial Sequence
  Antisense Oligonucleotide
tgcagactaggagtgaaagt

SEQ ID NO: 344 - DNA, 20 bp, Artificial Sequence
  Antisense Oligonucleotide
aggaggatgtccttttattg

SEQ ID NO: 345 - DNA, 20 bp, Artificial Sequence
  Antisense Oligonucleotide
atcagagcaccaaagggaat

SEQ ID NO: 346 - DNA, 20 bp, Artificial Sequence
  Antisense Oligonucleotide
ccagctcaacctgagaattc

SEQ ID NO: 347 - DNA, 20 bp, Artificial Sequence
  Antisense Oligonucleotide
catgacttacctggacatgg

SEQ ID NO: 348 - DNA, 20 bp, Artificial Sequence
  Antisense Oligonucleotide
cctcagcggacacacacaca

SEQ ID NO: 349 - DNA, 20 bp, Artificial Sequence
  Antisense Oligonucleotide
gtcacatccgtgcctggtgc

SEQ ID NO: 350 - DNA, 20 bp, Artificial Sequence
  Antisense Oligonucleotide
cagtgcctctgggaccccac

SEQ ID NO: 351 - DNA, 20 bp, Artificial Sequence
  Antisense Oligonucleotide
agctgcagtggccgatcagc

SEQ ID NO: 352 - DNA, 20 bp, Artificial Sequence
  Antisense Oligonucleotide
gacctccccagccacgtgga

SEQ ID NO: 353 - DNA, 20 bp, Artificial Sequence
  Antisense Oligonucleotide
tctgatcaccatacattaca

SEQ ID NO: 354 - DNA, 20 bp, Artificial Sequence
  Antisense Oligonucleotide
atttcccactgggtactctc

SEQ ID NO: 355 - DNA, 20 bp, Artificial Sequence
  Antisense Oligonucleotide
ggctgaagcccatgctgact

SEQ ID NO: 356 - DNA, 20 bp, Artificial Sequence
  Antisense Oligonucleotide
gttggacagtcattcttttg

SEQ ID NO: 357 - DNA, 20 bp, Artificial Sequence
  Antisense Oligonucleotide
cacttgttggacagtcattc

SEQ ID NO: 358 - DNA, 20 bp, Artificial Sequence
  Antisense Oligonucleotide
attttaaattacagtagata

SEQ ID NO: 359 - DNA, 20 bp, Artificial Sequence
  Antisense Oligonucleotide
ctgttctccacccatatcag

SEQ ID NO: 360 - DNA, 20 bp, Artificial Sequence
  Antisense Oligonucleotide
gagctcatacctgtcccaga

SEQ ID NO: 361 - DNA, 20 bp, Artificial Sequence
  Antisense Oligonucleotide
ttcaagggccactgctatca

SEQ ID NO: 362 - DNA, 20 bp, Artificial Sequence
  Antisense Oligonucleotide
ccagtatttcacgccaatcc

SEQ ID NO: 363 - DNA, 20 bp, Artificial Sequence
  Antisense Oligonucleotide
ggcaggaggaacctcgggca

SEQ ID NO: 364 - DNA, 20 bp, Artificial Sequence
  Antisense Oligonucleotide
ttttaaaattagacccaacc

SEQ ID NO: 365 - DNA, 20 bp, Artificial Sequence
  Antisense Oligonucleotide
tgactgttttaaaattagac

SEQ ID NO: 366 - DNA, 20 bp, Artificial Sequence
  Antisense Oligonucleotide
cccagcaaacacaggtgaag

SEQ ID NO: 367 - DNA, 20 bp, Artificial Sequence
  Antisense Oligonucleotide
gagtgtggtcttgctagtgc

SEQ ID NO: 368 - DNA, 20 bp, Artificial Sequence
  Antisense Oligonucleotide
ctatgcagagtgtggtcttg

SEQ ID NO: 369 - DNA, 20 bp, Artificial Sequence
  Antisense Oligonucleotide
agaagatgcaaccacatgta

SEQ ID NO: 370 - DNA, 20 bp, Artificial Sequence
  Antisense Oligonucleotide
acacggtatcctatggagga

SEQ ID NO: 371 - DNA, 20 bp, Artificial Sequence
  Antisense Oligonucleotide
tgggacttaccatgcctttg

SEQ ID NO: 372 - DNA, 20 bp, Artificial Sequence
  Antisense Oligonucleotide
ggttttgctgccctacatcc

SEQ ID NO: 373 - DNA, 20 bp, Artificial Sequence
  Antisense Oligonucleotide
acaaggagtccttgtgcaga

SEQ ID NO: 374 - DNA, 20 bp, Artificial Sequence
  Antisense Oligonucleotide
atgttcactgagacaggctg

SEQ ID NO: 375 - skipped

SEQ ID NO: 376 - DNA, 20 bp, Artificial Sequence
  Antisense Oligonucleotide
attagactggaagcatcctg

SEQ ID NO: 377 - DNA, 20 bp, Artificial Sequence
  Antisense Oligonucleotide
gagattggagacgagcattt

SEQ ID NO: 378 - DNA, 20 bp, Artificial Sequence
  Antisense Oligonucleotide
catgacctacttgtaggaga

SEQ ID NO: 379 - DNA, 20 bp, Artificial Sequence
  Antisense Oligonucleotide
tggatttggatacacaagtt

SEQ ID NO: 380 - DNA, 20 bp, Artificial Sequence
  Antisense Oligonucleotide
actcaatatatattcattga

SEQ ID NO: 381 - DNA, 20 bp, Artificial Sequence
  Antisense Oligonucleotide
caaggaagcacaccatgtca

SEQ ID NO: 382 - DNA, 20 bp, Artificial Sequence
  Antisense Oligonucleotide
atacttattcctggtaacca

SEQ ID NO: 383 - DNA, 20 bp, Artificial Sequence
  Antisense Oligonucleotide
ggtagccagaacaccagtgt

SEQ ID NO: 384 - DNA, 20 bp, Artificial Sequence
  Antisense Oligonucleotide
actagaggtagccagaacac

SEQ ID NO: 385 - DNA, 20 bp, Artificial Sequence
  Antisense Oligonucleotide
accacctgacatcacaggtt

SEQ ID NO: 386 - DNA, 20 bp, Artificial Sequence
  Antisense Oligonucleotide
tactgtgacctatgccagga

SEQ ID NO: 387 - DNA, 20 bp, Artificial Sequence
  Antisense Oligonucleotide
ggaggtgctactgttgacat

SEQ ID NO: 388 - DNA, 20 bp, Artificial Sequence
  Antisense Oligonucleotide
tccagacttgtctgagtcta

SEQ ID NO: 389 - DNA, 20 bp, Artificial Sequence
  Antisense Oligonucleotide
tctaagaggtagagctaaag

SEQ ID NO: 390 - DNA, 20 bp, Artificial Sequence
  Antisense Oligonucleotide
ccagagatgagcaacttagg

SEQ ID NO: 391 - DNA, 20 bp, Artificial Sequence
  Antisense Oligonucleotide
ggccatgtaaattgctcatc

SEQ ID NO: 392 - DNA, 20 bp, Artificial Sequence
  Antisense Oligonucleotide
aaagaaactatcctgtattc

SEQ ID NO: 393 - DNA, 20 bp, Artificial Sequence
  Antisense Oligonucleotide
ttcttagtacctggaagatg

SEQ ID NO: 394 - DNA, 20 bp, Artificial Sequence
  Antisense Oligonucleotide
cattagatacctggacacct

SEQ ID NO: 395 - DNA, 20 bp, Artificial Sequence
  Antisense Oligonucleotide
gtttcatggaactcagcgca

SEQ ID NO: 396 - DNA, 20 bp, Artificial Sequence
  Antisense Oligonucleotide
ctggagagcacctgcaatag

SEQ ID NO: 397 - DNA, 20 bp, Artificial Sequence
  Antisense Oligonucleotide
tgaagggtagagaaatcata

SEQ ID NO: 398 - DNA, 20 bp, Artificial Sequence
  Antisense Oligonucleotide
ggaaactcacttgttgaccg

SEQ ID NO: 399 - DNA, 20 bp, Artificial Sequence
  Antisense Oligonucleotide
aggtgcaagatgttcctctg

SEQ ID NO: 400 - DNA, 20 bp, Artificial Sequence
  Antisense Oligonucleotide
tgcacagaggtgcaagatgt

SEQ ID NO: 401 - DNA, 20 bp, Artificial Sequence
  Antisense Oligonucleotide
cacaagagtaaggagcagag

SEQ ID NO: 402 - DNA, 20 bp, Artificial Sequence
  Antisense Oligonucleotide
gatggatggtgagaaattac

SEQ ID NO: 403 - DNA, 20 bp, Artificial Sequence
  Antisense Oligonucleotide
tagacaattgagactcagaa

SEQ ID NO: 404 - DNA, 20 bp, Artificial Sequence
  Antisense Oligonucleotide
atgtgcacacaaggacatag

SEQ ID NO: 405 - DNA, 20 bp, Artificial Sequence
  Antisense Oligonucleotide
acatacaaatggcaataggc

SEQ ID NO: 406 - DNA, 20 bp, Artificial Sequence
  Antisense Oligonucleotide
taggcaaaggacatgaatag

SEQ ID NO: 407 - DNA, 20 bp, Artificial Sequence
  Antisense Oligonucleotide
ttatgatagctacagaataa

SEQ ID NO: 408 - DNA, 20 bp, Artificial Sequence
  Antisense Oligonucleotide
ctgagattacccgcagaatc

SEQ ID NO: 409 - DNA, 20 bp, Artificial Sequence
  Antisense Oligonucleotide
gatgtatgtcatataaaaga

SEQ ID NO: 410 - DNA, 20 bp, Artificial Sequence
  Antisense Oligonucleotide
tttccaatgacctgcattga

SEQ ID NO: 411 - DNA, 20 bp, Artificial Sequence
  Antisense Oligonucleotide
agggatggtcaatctggtag

SEQ ID NO: 412 - DNA, 20 bp, Artificial Sequence
  Antisense Oligonucleotide
ggctaataaatagggtagtt

SEQ ID NO: 413 - DNA, 20 bp, Artificial Sequence
  Antisense Oligonucleotide
tcctagagcactatcaagta

SEQ ID NO: 414 - DNA, 20 bp, Artificial Sequence
  Antisense Oligonucleotide
cctcctggtcctgcagtcaa

SEQ ID NO: 415 - DNA, 20 bp, Artificial Sequence
  Antisense Oligonucleotide
catttgcacaagtgtttgtt

SEQ ID NO: 416 - DNA, 20 bp, Artificial Sequence
  Antisense Oligonucleotide
ctgacacaccatgttattat

SEQ ID NO: 417 - skipped

SEQ ID NO: 418 - DNA, 20 bp, Artificial Sequence
  Antisense Oligonucleotide
tcacacttacctcgatgagg

SEQ ID NO: 419 - DNA, 20 bp, Artificial Sequence
  Antisense Oligonucleotide
aagaaaatggcatcaggttt

SEQ ID NO: 420 - DNA, 20 bp, Artificial Sequence
  Antisense Oligonucleotide
ccaagccaatctgagaaaga

SEQ ID NO: 421 - DNA, 20 bp, Artificial Sequence
  Antisense Oligonucleotide
aaatacacacctgctcatgt

SEQ ID NO: 422 - DNA, 20 bp, Artificial Sequence
  Antisense Oligonucleotide
cttcacaaatacacacctgc

SEQ ID NO: 423 - DNA, 20 bp, Artificial Sequence
  Antisense Oligonucleotide
agtggaagtttggtctcatt

SEQ ID NO: 424 - DNA, 20 bp, Artificial Sequence
  Antisense Oligonucleotide
ttgctagcttcaaagtggaa

SEQ ID NO: 425 - DNA, 20 bp, Artificial Sequence
  Antisense Oligonucleotide
tcaagaataagctccagatc

SEQ ID NO: 426 - DNA, 20 bp, Artificial Sequence
  Antisense Oligonucleotide
gcatacaagtcacatgaggt

SEQ ID NO: 427 - DNA, 20 bp, Artificial Sequence
  Antisense Oligonucleotide
tacaaggtgtttcttaagaa

SEQ ID NO: 428 - DNA, 20 bp, Artificial Sequence
  Antisense Oligonucleotide
atgcagccaggatgggccta

SEQ ID NO: 429 - DNA, 20 bp, Artificial Sequence
  Antisense Oligonucleotide
ttaccatatcctgagagttt

SEQ ID NO: 430 - DNA, 20 bp, Artificial Sequence
  Antisense Oligonucleotide
gcaaaggtagaggaaggtat

SEQ ID NO: 431 - DNA, 20 bp, Artificial Sequence
  Antisense Oligonucleotide
aaggaccttcagcaaaggta

SEQ ID NO: 432 - DNA, 20 bp, Artificial Sequence
  Antisense Oligonucleotide
cataggagtacatttatata

SEQ ID NO: 433 - DNA, 20 bp, Artificial Sequence
  Antisense Oligonucleotide
attatgataaaatcaatttt

SEQ ID NO: 434 - DNA, 20 bp, Artificial Sequence
  Antisense Oligonucleotide
agaaatttcactagatagat

SEQ ID NO: 435 - DNA, 20 bp, Artificial Sequence
  Antisense Oligonucleotide
agcatattttgatgagctga

SEQ ID NO: 436 - DNA, 20 bp, Artificial Sequence
  Antisense Oligonucleotide
gaaaggaaggactagcatat

SEQ ID NO: 437 - DNA, 20 bp, Artificial Sequence
  Antisense Oligonucleotide
cctctccaatctgtagaccc

SEQ ID NO: 438 - DNA, 20 bp, Artificial Sequence
  Antisense Oligonucleotide
ctggataactcagacctttg

SEQ ID NO: 439 - DNA, 20 bp, Artificial Sequence
  Antisense Oligonucleotide
agtcagaaaacaacctattc

SEQ ID NO: 440 - DNA, 20 bp, Artificial Sequence
  Antisense Oligonucleotide
cagcctgcatctataagtca

SEQ ID NO: 441 - DNA, 20 bp, Artificial Sequence
  Antisense Oligonucleotide
aaagaattaccctccactga

SEQ ID NO: 442 - DNA, 20 bp, Artificial Sequence
  Antisense Oligonucleotide
tctttcaaactggctaggca

SEQ ID NO: 443 - DNA, 20 bp, Artificial Sequence
  Antisense Oligonucleotide
gcctggcaaaattctgcagg

SEQ ID NO: 444 - DNA, 20 bp, Artificial Sequence
  Antisense Oligonucleotide
ctacctcaaatcaatatgtt

SEQ ID NO: 445 - DNA, 20 bp, Artificial Sequence
  Antisense Oligonucleotide
tgctttacctacctagctac

SEQ ID NO: 446 - DNA, 20 bp, Artificial Sequence
  Antisense Oligonucleotide
accttgtgtgtctcactcaa

SEQ ID NO: 447 - DNA, 20 bp, Artificial Sequence
  Antisense Oligonucleotide
atgcattccctgactagcac

SEQ ID NO: 448 - DNA, 20 bp, Artificial Sequence
  Antisense Oligonucleotide
catctctgagccccttacca

SEQ ID NO: 449 - DNA, 20 bp, Artificial Sequence
  Antisense Oligonucleotide
gctgggcatgctctctcccc

SEQ ID NO: 450 - DNA, 20 bp, Artificial Sequence
  Antisense Oligonucleotide
gctttcgcagctgggcatgc

SEQ ID NO: 451 - DNA, 20 bp, Artificial Sequence
  Antisense Oligonucleotide
actcctttctatacctggct

SEQ ID NO: 452 - DNA, 20 bp, Artificial Sequence
  Antisense Oligonucleotide
attctgcctcttagaaagtt

SEQ ID NO: 453 - DNA, 20 bp, Artificial Sequence
  Antisense Oligonucleotide
ccaagcctctttactgggct

SEQ ID NO: 454 - DNA, 20 bp, Artificial Sequence
  Antisense Oligonucleotide
cactcatgaccagactaaga

SEQ ID NO: 455 - DNA, 20 bp, Artificial Sequence
  Antisense Oligonucleotide
acctcccagaagccttccat

SEQ ID NO: 456 - DNA, 20 bp, Artificial Sequence
  Antisense Oligonucleotide
ttcatatgaaatctcctact

SEQ ID NO: 457 - DNA, 20 bp, Artificial Sequence
  Antisense Oligonucleotide
tatttaatttactgagaaac

SEQ ID NO: 458 - DNA, 20 bp, Artificial Sequence
  Antisense Oligonucleotide
taatgtgttgctggtgaaga

SEQ ID NO: 459 - DNA, 20 bp, Artificial Sequence
  Antisense Oligonucleotide
catctctaacctggtgtccc

SEQ ID NO: 460 - DNA, 20 bp, Artificial Sequence
  Antisense Oligonucleotide
gtgccatgctaggtggccat

SEQ ID NO: 461 - DNA, 20 bp, Artificial Sequence
  Antisense Oligonucleotide
agcaaattgggatctgtgct

SEQ ID NO: 462 - DNA, 20 bp, Artificial Sequence
  Antisense Oligonucleotide
tctggaggctcagaaacatg

SEQ ID NO: 463 - DNA, 20 bp, Artificial Sequence
  Antisense Oligonucleotide
tgaagacagggagccaccta

SEQ ID NO: 464 - DNA, 20 bp, Artificial Sequence
  Antisense Oligonucleotide
aggattcccaagactttgga

SEQ ID NO: 465 - DNA, 20 bp, Artificial Sequence
  Antisense Oligonucleotide
cagctctaatctaaagacat

SEQ ID NO: 466 - DNA, 20 bp, Artificial Sequence
  Antisense Oligonucleotide
gaatactcaccttctgcttg

SEQ ID NO: 467 - DNA, 20 bp, Artificial Sequence
  Antisense Oligonucleotide
atctctctgtcctcatcttc

SEQ ID NO: 468 - DNA, 20 bp, Artificial Sequence
  Antisense Oligonucleotide
ccaactccccctttctttgt

SEQ ID NO: 469 - DNA, 20 bp, Artificial Sequence
  Antisense Oligonucleotide
tctgggccaggaagacacga

SEQ ID NO: 470 - DNA, 20 bp, Artificial Sequence
  Antisense Oligonucleotide
tattgtgtgctgggcactgc

SEQ ID NO: 471 - DNA, 20 bp, Artificial Sequence
  Antisense Oligonucleotide
tgcttcgcacctggacgagt

SEQ ID NO: 472 - DNA, 20 bp, Artificial Sequence
  Antisense Oligonucleotide
ccttctttaccttaggtggc

SEQ ID NO: 473 - DNA, 20 bp, Artificial Sequence
  Antisense Oligonucleotide
gctctctctgccactctgat

SEQ ID NO: 474 - DNA, 20 bp, Artificial Sequence
  Antisense Oligonucleotide
aacttctaaagccaacattc

SEQ ID NO: 475 - DNA, 20 bp, Artificial Sequence
  Antisense Oligonucleotide
tgtgtcacaactatggtaaa

SEQ ID NO: 476 - DNA, 20 bp, Artificial Sequence
  Antisense Oligonucleotide
agacacataccataatgcca

SEQ ID NO: 477 - DNA, 20 bp, Artificial Sequence
  Antisense Oligonucleotide
ttctcttcatctgaaaatac

SEQ ID NO: 478 - DNA, 20 bp, Artificial Sequence
  Antisense Oligonucleotide
tgaggatgtaattagcactt

SEQ ID NO: 479 - DNA, 20 bp, Artificial Sequence
  Antisense Oligonucleotide
agctcattgcctacaaaatg

SEQ ID NO: 480 - DNA, 20 bp, Artificial Sequence
  Antisense Oligonucleotide
gttctcatgtttactaatgc

SEQ ID NO: 481 - DNA, 20 bp, Artificial Sequence
  Antisense Oligonucleotide
gaattgagacaacttgattt

SEQ ID NO: 482 - DNA, 20 bp, Artificial Sequence
  Antisense Oligonucleotide
ccggccatcgctgaaatgaa

SEQ ID NO: 483 - DNA, 20 bp, Artificial Sequence
  Antisense Oligonucleotide
catagctcaccttgcacatt

SEQ ID NO: 484 - DNA, 20 bp, Artificial Sequence
  Antisense Oligonucleotide
cggtgcaccctttacctgag

SEQ ID NO: 485 - DNA, 20 bp, Artificial Sequence
  Antisense Oligonucleotide
tctccagatcctaacataaa

SEQ ID NO: 486 - DNA, 20 bp, Artificial Sequence
  Antisense Oligonucleotide
ttgaatgacactagattttc

SEQ ID NO: 487 - DNA, 20 bp, Artificial Sequence
  Antisense Oligonucleotide
aaaatccattttctttaaag

SEQ ID NO: 488 - DNA, 20 bp, Artificial Sequence
  Antisense Oligonucleotide
cagctcacacttattttaaa

SEQ ID NO: 489 - DNA, 20 bp, Artificial Sequence
  Antisense Oligonucleotide
gttcccaaaactgtatagga

SEQ ID NO: 490 - DNA, 20 bp, Artificial Sequence
  Antisense Oligonucleotide
agctccatactgaagtcctt

SEQ ID NO: 491 - DNA, 20 bp, Artificial Sequence
  Antisense Oligonucleotide
caattcaataaaagctccat

SEQ ID NO: 492 - DNA, 20 bp, Artificial Sequence
  Antisense Oligonucleotide
gttttcaaaaggtataaggt

SEQ ID NO: 493 - DNA, 20 bp, Artificial Sequence
  Antisense Oligonucleotide
ttcccattccctgaaagcag

SEQ ID NO: 494 - DNA, 20 bp, Artificial Sequence
  Antisense Oligonucleotide
tggtatttacctgagggctg

SEQ ID NO: 495 - DNA, 20 bp, Artificial Sequence
  Antisense Oligonucleotide
ataaataatagtgctgatgg

SEQ ID NO: 496 - DNA, 20 bp, Artificial Sequence
  Antisense Oligonucleotide
ctatggctgagcttgcctat

SEQ ID NO: 497 - DNA, 20 bp, Artificial Sequence
  Antisense Oligonucleotide
ctctctgaaaaatataccct

SEQ ID NO: 498 - DNA, 20 bp, Artificial Sequence
  Antisense Oligonucleotide
ttgatgtatctcatctagca

SEQ ID NO: 499 - DNA, 20 bp, Artificial Sequence
  Antisense Oligonucleotide
tagaaccatgtttggtcttc

SEQ ID NO: 500 - DNA, 20 bp, Artificial Sequence
  Antisense Oligonucleotide
tttctctttatcacatgccc

SEQ ID NO: 501 - skipped

SEQ ID NO: 502 - DNA, 20 bp, Artificial Sequence
  Antisense Oligonucleotide
ctggagaggactaaacagag

SEQ ID NO: 503 - DNA, 568 bp, H. sapiens
  misc_feature 44, 99, 156, 468: n = A,T,C or G
ccaaaagattgattgactgtccattcaaagctacacgcaatttntgatatacatcacgtagttactgaaaaagctgcaatcaacacagttcatggaccnctaccatgaagcttgctccaggagaacttctatcattcctctaattttttaaaaganatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcataccactgaatgtggctcatttgtantaaaagacagtgaaacgagggcattgataaaatgttctggcacagcaaaacctctagaacacatagtgtgatttaagtaacagaataaaaatggaaacgg

SEQ ID NO: 504 - DNA, 20 bp, Artificial Sequence
  Antisense Oligonucleotide
acattttatcaatgccctcg

SEQ ID NO: 505 - DNA, 20 bp, Artificial Sequence
  Antisense Oligonucleotide
gccagaacattttatcaatg

SEQ ID NO: 506 - DNA, 20 bp, Artificial Sequence
  Antisense Oligonucleotide
agaggttttgctgtgccaga

SEQ ID NO: 507 - DNA, 20 bp, Artificial Sequence
  Antisense Oligonucleotide
ctagaggttttgctgtgcca

SEQ ID NO: 508 - DNA, 20 bp, Artificial Sequence
  Antisense Oligonucleotide
tctagaggttttgctgtgcc

SEQ ID NO: 509 - DNA, 20 bp, Artificial Sequence
  Antisense Oligonucleotide
aatcacactatgtgttctag

SEQ ID NO: 510 - DNA, 20 bp, Artificial Sequence
  Antisense Oligonucleotide
aaatcacactatgtgttcta

SEQ ID NO: 511 - skipped

SEQ ID NO: 512 - DNA, 20 bp, Artificial Sequence
  Antisense Oligonucleotide
cttaaatcacactatgtgtt

SEQ ID NO: 513 - DNA, 20 bp, Artificial Sequence
  Antisense Oligonucleotide
tattctgttacttaaatcac

SEQ ID NO: 514 - DNA, 20 bp, Artificial Sequence
  Antisense Oligonucleotide
tggtagcctcagtctgcttc

SEQ ID NO: 515 - DNA, 20 bp, Artificial Sequence
  Antisense Oligonucleotide
agtctgcttcgcgccttctg

SEQ ID NO: 516 - DNA, 20 bp, H. sapiens
gcgccagggccgaagaggaa

SEQ ID NO: 517 - DNA, 20 bp, H. sapiens
caggtatgagctcaagctgg

SEQ ID NO: 518 - DNA, 20 bp, H. sapiens
catcctgaacatcaagaggg

SEQ ID NO: 519 - DNA, 20 bp, H. sapiens
gggcagtgtgatcgcttcaa

SEQ ID NO: 520 - DNA, 20 bp, H. sapiens
cacttgctctcatcaaaggc

SEQ ID NO: 521 - DNA, 20 bp, H. sapiens
cacactggacgctaagagga

SEQ ID NO: 522 - DNA, 20 bp, H. sapiens
cgctgagccacgcggtcaac

SEQ ID NO: 523 - DNA, 20 bp, H. sapiens
tgtccaaattctaccatggg

SEQ ID NO: 524 - DNA, 20 bp, H. sapiens
cagctgacctcatcgagatt

SEQ ID NO: 525 - DNA, 20 bp, H. sapiens
gtcaagttcctgatggtgtc

SEQ ID NO: 526 - DNA, 20 bp, H. sapiens
agctgcttctgatgggtgcc

SEQ ID NO: 527 - DNA, 20 bp, H. sapiens
gggcatcatcattccggact

SEQ ID NO: 528 - DNA, 20 bp, H. sapiens
cctactatccgctgaccggg

SEQ ID NO: 529 - DNA, 20 bp, H. sapiens
gggccacctaagttgtgaca

SEQ ID NO: 530 - DNA, 20 bp, H. sapiens
agaacatgggattgccagac

SEQ ID NO: 531 - DNA, 20 bp, H. sapiens
ctccacttcaagtctgtggg

SEQ ID NO: 532 - DNA, 20 bp, H. sapiens
cagagcttggcctctctggg

SEQ ID NO: 533 - DNA, 20 bp, H. sapiens
tggccgcttcagggaacaca

SEQ ID NO: 534 - DNA, 20 bp, H. sapiens
cagctgagcagacaggcacc

SEQ ID NO: 535 - DNA, 20 bp, H. sapiens
gggagagacaagtttcacat

SEQ ID NO: 536 - DNA, 20 bp, H. sapiens
gtactgcatcctggattcaa

SEQ ID NO: 537 - DNA, 20 bp, H. sapiens
gtgaggtgactcagagactc

SEQ ID NO: 538 - DNA, 20 bp, H. sapiens
ttgcagagcaatattctatc

SEQ ID NO: 539 - DNA, 20 bp, H. sapiens
aagcattggtagagcaaggg

SEQ ID NO: 540 - DNA, 20 bp, H. sapiens
ccgctggctctgaaggagtc

SEQ ID NO: 541 - DNA, 20 bp, H. sapiens
tctagtcaggctgacctgcg

SEQ ID NO: 542 - DNA, 20 bp, H. sapiens
gggccacagtgttctaactg

SEQ ID NO: 543 - DNA, 20 bp, H. sapiens
aatcaagtgtcatcacactg

SEQ ID NO: 544 - DNA, 20 bp, H. sapiens
gggtagtcataacagtactg

SEQ ID NO: 545 - DNA, 20 bp, H. sapiens
agagcacacggtcttcagtg

SEQ ID NO: 546 - DNA, 20 bp, H. sapiens
ttacagctagagggcctctt

SEQ ID NO: 547 - DNA, 20 bp, H. sapiens
caccgtgggcatggatatgg

SEQ ID NO: 548 - DNA, 20 bp, H. sapiens
gggaatctgatgaggaaact

SEQ ID NO: 549 - DNA, 20 bp, H. sapiens
tgtcaacaagtaccactggg

SEQ ID NO: 550 - DNA, 20 bp, H. sapiens
acctgggatatacactaggg

SEQ ID NO: 551 - DNA, 20 bp, H. sapiens
ccaagtatagttggctggac

SEQ ID NO: 552 - DNA, 20 bp, H. sapiens
tacatgaagcttgctccagg

SEQ ID NO: 553 - DNA, 20 bp, H. sapiens
atgtcagcctggtctgtcca

SEQ ID NO: 554 - DNA, 20 bp, H. sapiens
gcacctccggaagtacacat

SEQ ID NO: 555 - DNA, 20 bp, H. sapiens
ctgcagcttcatcctgaaga

SEQ ID NO: 556 - DNA, 20 bp, H. sapiens
tgagggcaaagccttgctga

SEQ ID NO: 557 - DNA, 20 bp, H. sapiens
ccattccagaagggaagcag

SEQ ID NO: 558 - DNA, 20 bp, H. sapiens
cgaggaagggcaatgtggca

SEQ ID NO: 559 - DNA, 20 bp, H. sapiens
ccttgtcaactctgatcagc

SEQ ID NO: 560 - DNA, 20 bp, H. sapiens
agcagccagtcctgtcagta

SEQ ID NO: 561 - DNA, 20 bp, H. sapiens
agcatgtggcagaagccatc

SEQ ID NO: 562 - DNA, 20 bp, H. sapiens
gagagcaccaaatccacatc

SEQ ID NO: 563 - DNA, 20 bp, H. sapiens
cctcagtgatgaagcagtca

SEQ ID NO: 564 - DNA, 20 bp, H. sapiens
gatagatgtggtcacctacc

SEQ ID NO: 565 - DNA, 20 bp, H. sapiens
cctcagcacagcagctgcga

SEQ ID NO: 566 - DNA, 20 bp, H. sapiens
gattctgcgggtcattggaa

SEQ ID NO: 567 - DNA, 20 bp, H. sapiens
caaagccatcactgatgatc

SEQ ID NO: 568 - DNA, 20 bp, H. sapiens
agaaagctgccatccaggct

SEQ ID NO: 569 - DNA, 20 bp, H. sapiens
caggaggttcttcttcagac

SEQ ID NO: 570 - DNA, 20 bp, H. sapiens
gagtccttcacaggcagata

SEQ ID NO: 571 - DNA, 20 bp, H. sapiens
tgccaatatcttgaactcag

SEQ ID NO: 572 - DNA, 20 bp, H. sapiens
catcgagattggcttggaag

SEQ ID NO: 573 - DNA, 20 bp, H. sapiens
ggagctggattacagttgca

SEQ ID NO: 574 - DNA, 20 bp, H. sapiens
caacatgcaggctgaactgg

SEQ ID NO: 575 - DNA, 20 bp, H. sapiens
acattacatttggtctctac

SEQ ID NO: 576 - DNA, 20 bp, H. sapiens
ctcaggcgcttactccaacg

SEQ ID NO: 577 - DNA, 20 bp, H. sapiens
gggacaccagattagagctg

SEQ ID NO: 578 - DNA, 20 bp, H. sapiens
gagctccagagagaggacag

SEQ ID NO: 579 - DNA, 20 bp, H. sapiens
atcggcagagtatgaccttg

SEQ ID NO: 580 - DNA, 20 bp, H. sapiens
caagggtgttatttccatac

SEQ ID NO: 581 - DNA, 20 bp, H. sapiens
gactcatctgctacagctta

SEQ ID NO: 582 - DNA, 20 bp, H. sapiens
gcaaatcctccagagatcta

SEQ ID NO: 583 - DNA, 20 bp, H. sapiens
ctctcctgggtgttctagac

SEQ ID NO: 584 - DNA, 20 bp, H. sapiens
atgaaggctgactctgtggt

SEQ ID NO: 585 - DNA, 20 bp, H. sapiens
gggaccacagatgtctgctt

SEQ ID NO: 586 - DNA, 20 bp, H. sapiens
ctggccggctcaatggagag

SEQ ID NO: 587 - DNA, 20 bp, H. sapiens
gctgcgttctgaatatcagg

SEQ ID NO: 588 - DNA, 20 bp, H. sapiens
tgctgacatcttaggcactg

SEQ ID NO: 589 - DNA, 20 bp, H. sapiens
aagtgtagtctcctggtgct

SEQ ID NO: 590 - DNA, 20 bp, H. sapiens
caaaattcagtctggatggg

SEQ ID NO: 591 - DNA, 20 bp, H. sapiens
gggaaactacggctagaacc

SEQ ID NO: 592 - DNA, 20 bp, H. sapiens
ctgcatgtggctggtaacct

SEQ ID NO: 593 - DNA, 20 bp, H. sapiens
ccatgaccatcgatgcacat

SEQ ID NO: 594 - DNA, 20 bp, H. sapiens
atgggaaactcgctctctgg

SEQ ID NO: 595 - DNA, 20 bp, H. sapiens
agtcatcatctcgtgtctag

SEQ ID NO: 596 - DNA, 20 bp, H. sapiens
gaatacagccaggacttgga

SEQ ID NO: 597 - DNA, 20 bp, H. sapiens
ggcgtggagcttactggacg

SEQ ID NO: 598 - DNA, 20 bp, H. sapiens
gagatgagagatgccgttga

SEQ ID NO: 599 - DNA, 20 bp, H. sapiens
agtcttgatgagcactatca

SEQ ID NO: 600 - DNA, 20 bp, H. sapiens
ctaagtaccaaatcagaatc

SEQ ID NO: 601 - DNA, 20 bp, H. sapiens
gtccatgagttaatcgagag

SEQ ID NO: 602 - DNA, 20 bp, H. sapiens
aggccacagttgcagtgtat

SEQ ID NO: 603 - DNA, 20 bp, H. sapiens
tctgattggtggactcttgc

SEQ ID NO: 604 - DNA, 20 bp, H. sapiens
gaagtcagtcttcaggctct

SEQ ID NO: 605 - DNA, 20 bp, H. sapiens
ccagattctcagatgaggga

SEQ ID NO: 606 - DNA, 20 bp, H. sapiens
catctgtcattgatgcactg

SEQ ID NO: 607 - DNA, 20 bp, H. sapiens
aggagatgtcaagggttcgg

SEQ ID NO: 608 - DNA, 20 bp, H. sapiens
ggaactattgctagtgaggc

SEQ ID NO: 609 - DNA, 20 bp, H. sapiens
ctctctccatggcaaatgtc

SEQ ID NO: 610 - DNA, 20 bp, H. sapiens
caccgtgacttcagtgcaga

SEQ ID NO: 611 - DNA, 20 bp, H. sapiens
actgagttgagggtccggga

SEQ ID NO: 612 - DNA, 20 bp, H. sapiens
cacatatgaactggacctgc

SEQ ID NO: 613 - DNA, 20 bp, H. sapiens
tctgaactcagaaggatggc

SEQ ID NO: 614 - DNA, 20 bp, H. sapiens
ggtgcgaagcagactgaggc

SEQ ID NO: 615 - DNA, 20 bp, H. sapiens
tcccaccgggacctgcgggg

SEQ ID NO: 616 - DNA, 20 bp, H. sapiens
caccgggacctgcggggctg

SEQ ID NO: 617 - DNA, 20 bp, H. sapiens
ctgagtgcccttctcggttg

SEQ ID NO: 618 - DNA, 20 bp, H. sapiens
ctcggttgctgccgctgagg

SEQ ID NO: 619 - DNA, 20 bp, H. sapiens
tcggttgctgccgctgagga

SEQ ID NO: 620 - DNA, 20 bp, H. sapiens
cggttgctgccgctgaggag

SEQ ID NO: 621 - DNA, 20 bp, H. sapiens
gttgctgccgctgaggagcc

SEQ ID NO: 622 - DNA, 20 bp, H. sapiens
ctgccgctgaggagcccgcc

SEQ ID NO: 623 - DNA, 20 bp, H. sapiens
accgcagctggcgatggacc

SEQ ID NO: 624 - DNA, 20 bp, H. sapiens
cagctggcgatggacccgcc

SEQ ID NO: 625 - DNA, 20 bp, H. sapiens
gaacttactatcatcctcta

SEQ ID NO: 626 - DNA, 20 bp, H. sapiens
tccaattgaactttcacata

SEQ ID NO: 627 - DNA, 20 bp, H. sapiens
aaaattcaaactgcctatat

SEQ ID NO: 628 - DNA, 20 bp, H. sapiens
gataaaaccatacagtgagc

SEQ ID NO: 629 - DNA, 20 bp, H. sapiens
ataaaaccatacagtgagcc

SEQ ID NO: 630 - DNA, 20 bp, H. sapiens
aaccatacagtgagccagcc

SEQ ID NO: 631 - DNA, 20 bp, H. sapiens
accatacagtgagccagcct

SEQ ID NO: 632 - DNA, 20 bp, H. sapiens
ccatacagtgagccagcctt

SEQ ID NO: 633 - DNA, 20 bp, H. sapiens
gtgagccagccttgcagtag

SEQ ID NO: 634 - DNA, 20 bp, H. sapiens
ccagccttgcagtaggcagt

SEQ ID NO: 635 - DNA, 20 bp, H. sapiens
taggcagtagactataagca

SEQ ID NO: 636 - DNA, 20 bp, H. sapiens
gcagtagactataagcagaa

SEQ ID NO: 637 - DNA, 20 bp, H. sapiens
tgaactggacctgcaccaaa

SEQ ID NO: 638 - DNA, 20 bp, H. sapiens
ctggacctgcaccaaagctg

SEQ ID NO: 639 - DNA, 20 bp, H. sapiens
ggacctgcaccaaagctggc

SEQ ID NO: 640 - DNA, 20 bp, H. sapiens
ctgcaccaaagctggcacca

SEQ ID NO: 641 - DNA, 20 bp, H. sapiens
accaaagctggcaccagggc

SEQ ID NO: 642 - DNA, 20 bp, H. sapiens
ctcggaaggtctctgaactc

SEQ ID NO: 643 - DNA, 20 bp, H. sapiens
aactcagaaggatggcattt

SEQ ID NO: 644 - DNA, 20 bp, H. sapiens
ctcagaaggatggcattttt

SEQ ID NO: 645 - DNA, 20 bp, H. sapiens
atcaggatctgagttatttt

SEQ ID NO: 646 - DNA, 20 bp, H. sapiens
aggatctgagttattttgct

SEQ ID NO: 647 - DNA, 20 bp, H. sapiens
ctgagttattttgctaaact

SEQ ID NO: 648 - DNA, 20 bp, H. sapiens
attttgctaaacttggggga

SEQ ID NO: 649 - DNA, 20 bp, H. sapiens
taaacttgggggaggaggaa

SEQ ID NO: 650 - DNA, 20 bp, H. sapiens
ggaacaaataaatggagtct

SEQ ID NO: 651 - DNA, 20 bp, H. sapiens
gtttgtaactcaagcagaag

SEQ ID NO: 652 - DNA, 20 bp, H. sapiens
ttgtaactcaagcagaaggt

SEQ ID NO: 653 - DNA, 20 bp, H. sapiens
gtaactcaagcagaaggtgc

SEQ ID NO: 654 - DNA, 20 bp, H. sapiens
aactcaagcagaaggtgcga

SEQ ID NO: 655 - DNA, 20 bp, H. sapiens
ctcaagcagaaggtgcgaag

SEQ ID NO: 656 - DNA, 20 bp, H. sapiens
caagcagaaggtgcgaagca

SEQ ID NO: 657 - DNA, 20 bp, H. sapiens
agcagaaggtgcgaagcaga

SEQ ID NO: 658 - DNA, 20 bp, H. sapiens
cagaaggtgcgaagcagact

SEQ ID NO: 659 - DNA, 20 bp, H. sapiens
gaaggtgcgaagcagactga

SEQ ID NO: 660 - DNA, 20 bp, H. sapiens
aggtgcgaagcagactgagg

SEQ ID NO: 661 - DNA, 20 bp, H. sapiens
gtgcgaagcagactgaggct

SEQ ID NO: 662 - DNA, 20 bp, H. sapiens
gcgaagcagactgaggctac

SEQ ID NO: 663 - DNA, 20 bp, H. sapiens
gaagcagactgaggctacca

SEQ ID NO: 664 - DNA, 20 bp, H. sapiens
agcagactgaggctaccatg

SEQ ID NO: 665 - DNA, 20 bp, H. sapiens
cagactgaggctaccatgac

SEQ ID NO: 666 - DNA, 20 bp, H. sapiens
gactgaggctaccatgacat

SEQ ID NO: 667 - DNA, 20 bp, H. sapiens
ctgaggctaccatgacattc

SEQ ID NO: 668 - DNA, 20 bp, H. sapiens
gaggctaccatgacattcaa

SEQ ID NO: 669 - DNA, 20 bp, H. sapiens
ggctaccatgacattcaaat

SEQ ID NO: 670 - DNA, 20 bp, H. sapiens
ctaccatgacattcaaatat

SEQ ID NO: 671 - DNA, 20 bp, H. sapiens
cctgaagctgcatgtggctg

SEQ ID NO: 672 - DNA, 20 bp, H. sapiens
tgaagctgcatgtggctggt

SEQ ID NO: 673 - DNA, 20 bp, H. sapiens
aagctgcatgtggctggtaa

SEQ ID NO: 674 - DNA, 20 bp, H. sapiens
gctgcatgtggctggtaacc

SEQ ID NO: 675 - DNA, 20 bp, H. sapiens
tgcatgtggctggtaaccta

SEQ ID NO: 676 - DNA, 20 bp, H. sapiens
catgtggctggtaacctaaa

SEQ ID NO: 677 - DNA, 20 bp, H. sapiens
tgtggctggtaacctaaaag

SEQ ID NO: 678 - DNA, 20 bp, H. sapiens
tggctggtaacctaaaagga

SEQ ID NO: 679 - DNA, 20 bp, H. sapiens
gctggtaacctaaaaggagc

SEQ ID NO: 680 - DNA, 20 bp, H. sapiens
tggtaacctaaaaggagcct

SEQ ID NO: 681 - DNA, 20 bp, H. sapiens
gtaacctaaaaggagcctac

SEQ ID NO: 682 - DNA, 20 bp, H. sapiens
aacctaaaaggagcctacca

SEQ ID NO: 683 - DNA, 20 bp, H. sapiens
cctaaaaggagcctaccaaa

SEQ ID NO: 684 - DNA, 20 bp, H. sapiens
ggcgcgaagcagactgaggc

SEQ ID NO: 685 - DNA, 20 bp, H. sapiens
cactatgttcatgagggagg

SEQ ID NO: 686 - DNA, 20 bp, H. sapiens
ccatcataggttctgacgtc

SEQ ID NO: 687 - DNA, 20 bp, H. sapiens
gaagctgattgactcactca

SEQ ID NO: 688 - DNA, 20 bp, H. sapiens
ttgtaactcaagcagaaggc

SEQ ID NO: 689 - DNA, 20 bp, H. sapiens
gtaactcaagcagaaggcgc

SEQ ID NO: 690 - DNA, 20 bp, H. sapiens
aactcaagcagaaggcgcga

SEQ ID NO: 691 - DNA, 20 bp, H. sapiens
ctcaagcagaaggcgcgaag

SEQ ID NO: 692 - DNA, 20 bp, H. sapiens
cagaaggcgcgaagcagact

SEQ ID NO: 693 - DNA, 20 bp, H. sapiens
gaaggcgcgaagcagactga

SEQ ID NO: 694 - DNA, 20 bp, H. sapiens
aggcgcgaagcagactgagg

SEQ ID NO: 695 - DNA, 20 bp, H. sapiens
gcgcgaagcagactgaggct

SEQ ID NO: 696 - DNA, 20 bp, H. sapiens
gaagcagactgaggctacca

SEQ ID NO: 697 - DNA, 20 bp, H. sapiens
cagaaggcgcgaagcagact

SEQ ID NO: 698 - DNA, 20 bp, H. sapiens
tctttctcctgtcttacaga

SEQ ID NO: 699 - DNA, 20 bp, H. sapiens
cccacgttagaagatgcgac

SEQ ID NO: 700 - DNA, 20 bp, H. sapiens
aatatggtagacatgagcca

SEQ ID NO: 701 - DNA, 20 bp, H. sapiens
cattagctgcatttcaactg

SEQ ID NO: 702 - DNA, 20 bp, H. sapiens
actttcactcctagtctgca

SEQ ID NO: 703 - DNA, 20 bp, H. sapiens
ccatgtccaggtaagtcatg

SEQ ID NO: 704 - DNA, 20 bp, H. sapiens
gcaccaggcacggatgtgac

SEQ ID NO: 705 - DNA, 20 bp, H. sapiens
gtggggtcccagaggcactg

SEQ ID NO: 706 - DNA, 20 bp, H. sapiens
gctgatcggccactgcagct

SEQ ID NO: 707 - DNA, 20 bp, H. sapiens
tccacgtggctggggaggtc

SEQ ID NO: 708 - DNA, 20 bp, H. sapiens
tgtaatgtatggtgatcaga

SEQ ID NO: 709 - DNA, 20 bp, H. sapiens
gagagtacccagtgggaaat

SEQ ID NO: 710 - DNA, 20 bp, H. sapiens
agtcagcatgggcttcagcc

SEQ ID NO: 711 - DNA, 20 bp, H. sapiens
caaaagaatgactgtccaac

SEQ ID NO: 712 - DNA, 20 bp, H. sapiens
gaatgactgtccaacaagtg

SEQ ID NO: 713 - DNA, 20 bp, H. sapiens
tatctactgtaatttaaaat

SEQ ID NO: 714 - DNA, 20 bp, H. sapiens
ctgatatgggtggagaacag

SEQ ID NO: 715 - DNA, 20 bp, H. sapiens
tctgggacaggtatgagctc

SEQ ID NO: 716 - DNA, 20 bp, H. sapiens
tgatagcagtggcccttgaa

SEQ ID NO: 717 - DNA, 20 bp, H. sapiens
ggattggcgtgaaatactgg

SEQ ID NO: 718 - DNA, 20 bp, H. sapiens
tgcccgaggttcctcctgcc

SEQ ID NO: 719 - DNA, 20 bp, H. sapiens
gcactagcaagaccacactc

SEQ ID NO: 720 - DNA, 20 bp, H. sapiens
caagaccacactctgcatag

SEQ ID NO: 721 - DNA, 20 bp, H. sapiens
tcctccataggataccgtgt

SEQ ID NO: 722 - DNA, 20 bp, H. sapiens
ggatgtagggcagcaaaacc

SEQ ID NO: 723 - DNA, 20 bp, H. sapiens
tctgcacaaggactccttgt

SEQ ID NO: 724 - DNA, 20 bp, H. sapiens
cagcctgtctcagtgaacat

SEQ ID NO: 725 - DNA, 20 bp, H. sapiens
caggatgcttccagtctaat

SEQ ID NO: 726 - DNA, 20 bp, H. sapiens
aaatgctcgtctccaatctc

SEQ ID NO: 727 - DNA, 20 bp, H. sapiens
aacttgtgtatccaaatcca

SEQ ID NO: 728 - DNA, 20 bp, H. sapiens
tgacatggtgtgcttccttg

SEQ ID NO: 729 - DNA, 20 bp, H. sapiens
acactggtgttctggctacc

SEQ ID NO: 730 - DNA, 20 bp, H. sapiens
gtgttctggctacctctagt

SEQ ID NO: 731 - DNA, 20 bp, H. sapiens
tcctggcataggtcacagta

SEQ ID NO: 732 - DNA, 20 bp, H. sapiens
atgtcaacagtagcacctcc

SEQ ID NO: 733 - DNA, 20 bp, H. sapiens
tagactcagacaagtctgga

SEQ ID NO: 734 - DNA, 20 bp, H. sapiens
cctaagttgctcatctctgg

SEQ ID NO: 735 - DNA, 20 bp, H. sapiens
tgcgctgagttccatgaaac

SEQ ID NO: 736 - DNA, 20 bp, H. sapiens
ctattgcaggtgctctccag

SEQ ID NO: 737 - DNA, 20 bp, H. sapiens
cagaggaacatcttgcacct

SEQ ID NO: 738 - DNA, 20 bp, H. sapiens
ctctgctccttactcttgtg

SEQ ID NO: 739 - DNA, 20 bp, H. sapiens
gtaatttctcaccatccatc

SEQ ID NO: 740 - DNA, 20 bp, H. sapiens
ttctgagtctcaattgtcta

SEQ ID NO: 741 - DNA, 20 bp, H. sapiens
ctatgtccttgtgtgcacat

SEQ ID NO: 742 - DNA, 20 bp, H. sapiens
gcctattgccatttgtatgt

SEQ ID NO: 743 - DNA, 20 bp, H. sapiens
ctattcatgtcctttgccta

SEQ ID NO: 744 - DNA, 20 bp, H. sapiens
gattctgcgggtaatctcag

SEQ ID NO: 745 - DNA, 20 bp, H. sapiens
tcaatgcaggtcattggaaa

SEQ ID NO: 746 - DNA, 20 bp, H. sapiens
ctaccagattgaccatccct

SEQ ID NO: 747 - DNA, 20 bp, H. sapiens
tacttgatagtgctctagga

SEQ ID NO: 748 - DNA, 20 bp, H. sapiens
ttgactgcaggaccaggagg

SEQ ID NO: 749 - DNA, 20 bp, H. sapiens
aacaaacacttgtgcaaatg

SEQ ID NO: 750 - DNA, 20 bp, H. sapiens
aatgagaccaaacttccact

SEQ ID NO: 751 - DNA, 20 bp, H. sapiens
ttccactttgaagctagcaa

SEQ ID NO: 752 - DNA, 20 bp, H. sapiens
gatctggagcttattcttga

SEQ ID NO: 753 - DNA, 20 bp, H. sapiens
acctcatgtgacttgtatgc

SEQ ID NO: 754 - DNA, 20 bp, H. sapiens
ttcttaagaaacaccttgta

SEQ ID NO: 755 - DNA, 20 bp, H. sapiens
taggcccatcctggctgcat

SEQ ID NO: 756 - DNA, 20 bp, H. sapiens
aaactctcaggatatggtaa

SEQ ID NO: 757 - DNA, 20 bp, H. sapiens
ataccttcctctacctttgc

SEQ ID NO: 758 - DNA, 20 bp, H. sapiens
tacctttgctgaaggtcctt

SEQ ID NO: 759 - DNA, 20 bp, H. sapiens
atctatctagtgaaatttct

SEQ ID NO: 760 - DNA, 20 bp, H. sapiens
tcagctcatcaaaatatgct

SEQ ID NO: 761 - DNA, 20 bp, H. sapiens
atatgctagtccttcctttc

SEQ ID NO: 762 - DNA, 20 bp, H. sapiens
caaaggtctgagttatccag

SEQ ID NO: 763 - DNA, 20 bp, H. sapiens
tgacttatagatgcaggctg

SEQ ID NO: 764 - DNA, 20 bp, H. sapiens
tcagtggagggtaattcttt

SEQ ID NO: 765 - DNA, 20 bp, H. sapiens
tgcctagccagtttgaaaga

SEQ ID NO: 766 - DNA, 20 bp, H. sapiens
cctgcagaattttgccaggc

SEQ ID NO: 767 - DNA, 20 bp, H. sapiens
gtagctaggtaggtaaagca

SEQ ID NO: 768 - DNA, 20 bp, H. sapiens
ttgagtgagacacacaaggt

SEQ ID NO: 769 - DNA, 20 bp, H. sapiens
gtgctagtcagggaatgcat

SEQ ID NO: 770 - DNA, 20 bp, H. sapiens
ggggagagagcatgcccagc

SEQ ID NO: 771 - DNA, 20 bp, H. sapiens
gcatgcccagctgcgaaagc

SEQ ID NO: 772 - DNA, 20 bp, H. sapiens
agccaggtatagaaaggagt

SEQ ID NO: 773 - DNA, 20 bp, H. sapiens
aactttctaagaggcagaat

SEQ ID NO: 774 - DNA, 20 bp, H. sapiens
tcttagtctggtcatgagtg

SEQ ID NO: 775 - DNA, 20 bp, H. sapiens
agtaggagatttcatatgaa

SEQ ID NO: 776 - DNA, 20 bp, H. sapiens
tcttcaccagcaacacatta

SEQ ID NO: 777 - DNA, 20 bp, H. sapiens
atggccacctagcatggcac

SEQ ID NO: 778 - DNA, 20 bp, H. sapiens
catgtttctgagcctccaga

SEQ ID NO: 779 - DNA, 20 bp, H. sapiens
taggtggctccctgtcttca

SEQ ID NO: 780 - DNA, 20 bp, H. sapiens
tccaaagtcttgggaatcct

SEQ ID NO: 781 - DNA, 20 bp, H. sapiens
acaaagaaagggggagttgg

SEQ ID NO: 782 - DNA, 20 bp, H. sapiens
tcgtgtcttcctggcccaga

SEQ ID NO: 783 - DNA, 20 bp, H. sapiens
gcagtgcccagcacacaata

SEQ ID NO: 784 - DNA, 20 bp, H. sapiens
actcgtccaggtgcgaagca

SEQ ID NO: 785 - DNA, 20 bp, H. sapiens
gccacctaaggtaaagaagg

SEQ ID NO: 786 - DNA, 20 bp, H. sapiens
atcagagtggcagagagagc

SEQ ID NO: 787 - DNA, 20 bp, H. sapiens
tttaccatagttgtgacaca

SEQ ID NO: 788 - DNA, 20 bp, H. sapiens
cattttgtaggcaatgagct

SEQ ID NO: 789 - DNA, 20 bp, H. sapiens
gcattagtaaacatgagaac

SEQ ID NO: 790 - DNA, 20 bp, H. sapiens
ttcatttcagcgatggccgg

SEQ ID NO: 791 - DNA, 20 bp, H. sapiens
gaaaatctagtgtcattcaa

SEQ ID NO: 792 - DNA, 20 bp, H. sapiens
tcctatacagttttgggaac

SEQ ID NO: 793 - DNA, 20 bp, H. sapiens
aaggacttcagtatggagct

SEQ ID NO: 794 - DNA, 20 bp, H. sapiens
atggagcttttattgaattg

SEQ ID NO: 795 - DNA, 20 bp, H. sapiens
ccatcagcactattatttat

SEQ ID NO: 796 - DNA, 20 bp, H. sapiens
ataggcaagctcagccatag

SEQ ID NO: 797 - DNA, 20 bp, H. sapiens
tgctagatgagatacatcaa

SEQ ID NO: 798 - DNA, 20 bp, H. sapiens
gaagaccaaacatggttcta

SEQ ID NO: 799 - DNA, 20 bp, H. sapiens
ctctgtttagtcctctccag

SEQ ID NO: 800 - DNA, 20 bp, H. sapiens
cattgataaaatgttctggc

SEQ ID NO: 801 - DNA, 20 bp, H. sapiens
tctggcacagcaaaacctct

SEQ ID NO: 802 - DNA, 20 bp, H. sapiens
tggcacagcaaaacctctag

SEQ ID NO: 803 - DNA, 20 bp, H. sapiens
tagaacacatagtgtgattt

SEQ ID NO: 804 - DNA, 20 bp, H. sapiens
aacacatagtgtgatttaag

SEQ ID NO: 805-814 - skipped (10 entries)

SEQ ID NO: 815 - DNA, 20 bp, Artificial Sequence
  Antisense Oligonucleotide
gcgttgtctccgatgttctg

SEQ ID NO: 816 - DNA, 20 bp, Artificial Sequence
  Antisense Oligonucleotide
taatcattaacttgctgtgg

SEQ ID NO: 817-818 - skipped (2 entries)

SEQ ID NO: 819 - DNA, 20 bp, Artificial Sequence
  Antisense Oligonucleotide
caattgaatgtactcagata

SEQ ID NO: 820 - DNA, 20 bp, Artificial Sequence
  Antisense Oligonucleotide
acctcagtgacttgtaatca

SEQ ID NO: 821 - DNA, 20 bp, Artificial Sequence
  Antisense Oligonucleotide
cactggaaacttgtctctcc

SEQ ID NO: 822-823 - skipped (2 entries)

SEQ ID NO: 824 - DNA, 20 bp, Artificial Sequence
  Antisense Oligonucleotide
attggaataatgtatccagg

SEQ ID NO: 825 - DNA, 20 bp, Artificial Sequence
  Antisense Oligonucleotide
ttggcattatccaatgcagt

SEQ ID NO: 826 - skipped

SEQ ID NO: 827 - DNA, 20 bp, Artificial Sequence
  Antisense Oligonucleotide
attgtgagtggagatacttc

SEQ ID NO: 828 - DNA, 20 bp, Artificial Sequence
  Antisense Oligonucleotide
catatgtctgaagttgagac

SEQ ID NO: 829 - skipped

SEQ ID NO: 830 - DNA, 20 bp, Artificial Sequence
  Antisense Oligonucleotide
ggatcacatgactgaatgct

SEQ ID NO: 831 - DNA, 20 bp, Artificial Sequence
  Antisense Oligonucleotide
tcaagctggttgttgcactg

SEQ ID NO: 832 - skipped

SEQ ID NO: 833 - DNA, 20 bp, Artificial Sequence
  Antisense Oligonucleotide
gctcattctccagcatcagg

SEQ ID NO: 834 - DNA, 20 bp, Artificial Sequence
  Antisense Oligonucleotide
ttgatctataatactagcta

SEQ ID NO: 835 - DNA, 20 bp, Artificial Sequence
  Antisense Oligonucleotide
atggaagactggcagctcta

SEQ ID NO: 836 - skipped

SEQ ID NO: 837 - DNA, 20 bp, Artificial Sequence
  Antisense Oligonucleotide
tgtgcacggatatgataacg

SEQ ID NO: 838 - DNA, 20 bp, Artificial Sequence
  Antisense Oligonucleotide
gaccttgagtagattcctgg

SEQ ID NO: 839 - DNA, 20 bp, Artificial Sequence
  Antisense Oligonucleotide
gaaatctggaagagagacct

SEQ ID NO: 840-841 - skipped (2 entries)

SEQ ID NO: 842 - DNA, 20 bp, Artificial Sequence
  Antisense Oligonucleotide
atgttgcccatggctggaat

SEQ ID NO: 843 - DNA, 20 bp, Artificial Sequence
  Antisense Oligonucleotide
aagatgcagtactacttcca

SEQ ID NO: 844 - skipped

SEQ ID NO: 845 - DNA, 20 bp, Artificial Sequence
  Antisense Oligonucleotide
cttgatacttggtatccaca

SEQ ID NO: 846 - DNA, 20 bp, Artificial Sequence
  Antisense Oligonucleotide
cagtgtaatgatcgttgatt

SEQ ID NO: 847 - DNA, 20 bp, Artificial Sequence
  Antisense Oligonucleotide
taaagtccagcattggtatt

SEQ ID NO: 848 - DNA, 20 bp, Artificial Sequence
  Antisense Oligonucleotide
caacaatgtctgattggtta

SEQ ID NO: 849 - DNA, 20 bp, Artificial Sequence
  Antisense Oligonucleotide
gaagaggaagaaaggatatg

SEQ ID NO: 850 - DNA, 20 bp, Artificial Sequence
  Antisense Oligonucleotide
tgacagatgaagaggaagaa

SEQ ID NO: 851 - DNA, 20 bp, Artificial Sequence
  Antisense Oligonucleotide
ttgtactgtagtgcatcaat

SEQ ID NO: 852 - DNA, 20 bp, Artificial Sequence
  Antisense Oligonucleotide
gcctcaatctgttgtttcag

SEQ ID NO: 853 - DNA, 20 bp, Artificial Sequence
  Antisense Oligonucleotide
acttgagcgtgccctctaat

SEQ ID NO: 854 - DNA, 20 bp, Artificial Sequence
  Antisense Oligonucleotide
gaaatggaattgtagttctc